(19) United States
(12) Patent Application Publication
(10) Pub. No.: US 2010/0041700 A1
(43) Pub. Date: Feb. 18, 2010

(54) Substituted 1-(Azolin-2-yl)-Amino-1,2-Heterocyclyl-Ethane Compounds For Combating Pests

(75) Inventors: Markus Kordes, Bobenheim-Roxheim (DE); Ronan Le Vezouet, Mannheim (DE); Christopher Koradin, Ludwigshafen (DE); Ernst Baumann, Dudenhofen (DE); Deborah L. Culbertson, Fuquay Varina, NC (US)

Correspondence Address: BRINKS, HOFER, GILSON & LIONE, P.O. BOX 1340, MORRISVILLE, NC, 27560, US

(73) Assignee: BASF SE, Ludwigshafen (DE)

(21) Appl. No.: 12/519,985

(22) PCT Filed: Dec. 11, 2007
(86) PCT No.: PCT/EP2007/063654

§ 371 Date: Jun. 18, 2009

Related U.S. Application Data
(60) Provisional application No. 60/871,279, filed on Dec. 21, 2006.

(51) Int. Cl. A01N 43/78; C07D 417/14; A01N 43/42; A01P 7/04; A01P 5/00

(52) U.S. Cl. 514/314; 546/270.7; 546/165; 546/256; 546/176; 514/342

(57) ABSTRACT

The present invention relates to 1-(Azolin-2-yl)-amino-1,2-heterocyclyl-ethane compounds, which are useful for combating insects, arachnids and nematodes. The present invention also relates to methods for combating animal pests and to compositions for combating animal pests. It has been found that animal pests can be combated by 1-(Azolin-2-yl)-amino-1,2-heterocyclyl-ethane compounds of the general formula (I): wherein A is a radical of the formulae A¹ or A²: and wherein X is sulfur, oxygen or NR⁷, and Het_{A}, Het_{B} and R¹ to R⁷ are defined as in the description.

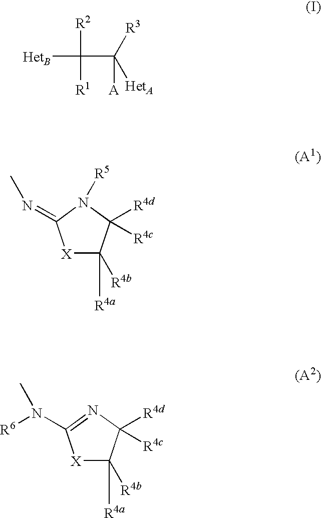

The present invention relates to 1-(azolin-2-yl)-amino-1,2-heterocyclyl-ethane compounds and 1-(aminothiocarbonylamino)-1,2-heterocyclyl-ethane compounds and their enantiomers, diastereomers and salts, including agricultural compositions comprising thereof, which are useful for combating animal pest, in particular insects, arachnids and nematodes. The present invention also relates to a method for combating such pests and for protecting crops against infestation or infection by such pests. The present invention relates further to a method for treating and protecting seeds against infestation or infection by such pests. Furthermore, the present invention relates to veterinary compositions for combating animal pests.

Animal pests and in particular insects, arachnids and nematodes destroy growing and harvested crops and attack wooden dwelling and commercial structures, causing large economic loss to the food supply and to property. While a large number of pesticidal agents are known, due to the ability of target pests to develop resistance to said agents, there is an ongoing need for new agents for combating insects, arachnids and nematodes.

WO 2005/063724 and unpublished U.S. application Ser. No. 60/958,134 describe 1-(azolin-2-yl)amino-1,2-diphenylethane compounds which are useful for combating insects, arachnids and nematodes. For the same purpose 1-(azolin-2-yl)-amino-2-aryl-1-hetaryl-ethane compounds are disclosed in WO2007/071585.

Unpublished U.S. application Ser. No. 60/817,973 disclose 1-(azolin-2-yl)-amino-1-phenyl-2-hetaryl-ethane compounds for combating insects, arachnids and nematodes.

N-substituted 1-thiazol-amino-alkane compounds have been described for their ubiquitin ligase inhibitory activity in pharmaceutical compositions in WO2006/074262.

However, the pesticidal action of the compounds disclosed in some of the above-mentioned literature references is not always completely satisfying.

It is therefore an object of the present invention to provide compounds having a good pesticidal activity and showing a broad activity spectrum against a large number of different animal pests, especially against difficult to control insects, arachnids and nematodes.

It has been found that these objectives can be achieved by 1-(azolin-2-yl)-amino-1,2-heterocyclyl-ethane compounds of the general formula I:

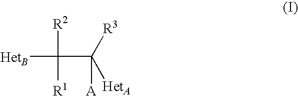

wherein
R¹, R², R³ are, independently of each other, selected from hydrogen, halogen, C₁-C₆-alkyl, C₂-C₆-alkenyl, C₂-C₆-alkynyl, C₁-C₆-haloalkyl, C₃-C₆-cycloalkyl, C₃-C₆-halocycloalkyl,
   wherein 1, 2 or 3 hydrogen atoms in the aforementioned aliphatic radicals may be replaced, independently of one another, by a radical selected from the group consisting of CN, NO₂, OH, SH, NH₂, CO₂H, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-haloalkoxy and C₁-C₆-alkylthio, and
   wherein 1, 2, 3, 4 or 5 hydrogen atoms of the C₃-C₆-cycloalkyl and C₃-C₆-halocycloalkyl may be replaced by radicals selected from C₁-C₆-alkyl and C₁-C₆-haloalkyl;
phenyl or benzyl, wherein the phenyl ring in the last two mentioned radicals may be unsubstituted or may carry 1, 2, 3, 4 or 5 radicals which are, independently of each other, by a radical selected from the group consisting of halogen, C₁-C₆-alkyl, C₁-C₆-haloalkyl, C₁-C₆-alkylthio, C₁-C₆-haloalkylthio, C₁-C₆-alkoxy and C₁-C₆-haloalkoxy;
A is a radical of the formulae A¹ or A²:

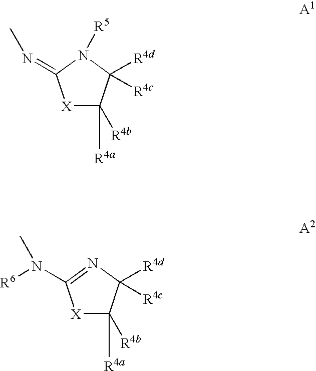

wherein
   X is sulfur, oxygen or NR⁷;
   R^{4a}, R^{4b}, R^{4c}, R^{4d} are, independently of each other, selected from the group consisting of hydrogen, halogen, C₁-C₆-alkyl, C₁-C₆-haloalkyl, C₁-C₆— alkylamino, C₁-C₆-alkoxy, C₁-C₆-haloalkoxy, C₃-C₆-cycloalkyl and C₃-C₆-halocycloalkyl,
   wherein 1, 2 or 3 hydrogen atoms in the aforementioned aliphatic radicals may be replaced, independently of one another, by a radical selected from the group consisting of CN, NO₂, OH, SH, NH₂, CO₂H, C₂-C₆-alkenyl, C₂-C₆-alkynyl, C₁-C₆-alkoxy, C₂-C₆-alkenyloxy, C₂-C₆-alkynyloxy, C₁-C₆-haloalkoxyl and C₁-C₆-alkylthio and
   wherein 1, 2, 3, 4 or 5 hydrogen atoms of the C₃-C₆-cycloalkyl and C₃-C₆-halocycloalkyl may be replaced by radicals selected from C₁-C₆-alkyl and C₁-C₆-haloalkyl;
R⁵, R⁶, R⁷, R⁹ are, independently of each other, selected from the group consisting of hydrogen, CN, NO₂, C₁-C₆-alkyl, C₂-C₆-alkenyl, C₂-C₆-alkynyl, C₃-C₈-cycloalkyl, C₃-C₈-halocycloalkyl, C₁-C₆-alkoxy, (C₁-C₆-alkoxy)methylen, C₁-C₆-alkylthio, C₁-C₆-alkylsulfinyl, C₁-C₆-alkylsulfonyl,
   wherein the aliphatic moieties in the aforementioned radicals may be unsubstituted, partially or completely halogenated and/or may carry 1, 2 or 3 radicals, which are independently of one another, selected from the group consisting of CN, NO₂, OH, SH, NH₂, CO₂H, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-haloalkoxy and C₁-C₆-alkylthio and
   wherein 1, 2, 3, 4 or 5 hydrogen atoms of the C₃-C₈-cycloalkyl and C₃-C₈-halocycloalkyl may replaced by radicals selected from C₁-C₆-alkyl and C₁-C₆-haloalkyl;
C(O)NR^{a}R^{b}, C(S)NR^{a}R^{b}, (SO₂)NR^{a}R^{b}, C(═O)R^{c} or C(═S)R^{c},
phenyl, phenyloxy or benzyl, wherein the phenyl ring in each of the last three mentioned radicals may be unsubstituted or may carry 1, 2, 3, 4 or 5 radicals, independently of one another selected from the group consisting of halogen, C₁-C₆-alkyl, C₁-C₆-haloalkyl, C₁-C₆-alkylthio, C₁-C₆-haloalkylthio, C₁-C₆-alkoxy and C₁-C₆-haloalkoxy radicals;
Het_{A}, Het_{B} are, independently of each other, selected from a 5-, 6- or 7-membered saturated, partially unsaturated or aromatic heterocyclic ring which contains 1, 2, 3 or 4 heteroatoms selected from oxygen, nitrogen and sulfur as ring members, wherein the heterocyclic ring may optionally be fused to another ring selected from phenyl, a saturated or partially unsaturated 5-, 6-, or 7-membered carbocycle or a 5-, 6- or 7-membered saturated, partially unsaturated or aromatic heterocycle, which contains 1, 2 or 3 heteroatoms selected from oxygen, sulfur and nitrogen atoms as ring members,
and wherein the 5-, 6- or 7-membered heterocyclic ring and/or the respective fused ring may carry at its carbon atoms any combination of m radicals R⁸ and/or may carry at its nitrogen atom, if present, a radical R⁹, which is as defined above or oxygen, and wherein
m is 0, 1, 2, 3 or 4, and
R⁸ is selected independently from m from halogen, OH, SH, NH₂, SO₃H, COOH, CN, N₃, NO₂, CONH₂, CSNH₂, CH═N—OH, CH═N—O—(C₁-C₆)-alkyl, C₁-C₆-alkyl, C₂-C₆-alkenyl, C₂-C₆-alkynyl, C₃-C₈-cycloalkyl, C₁-C₆-alkylamino, C₂-C₆-alkenylamino, C₂-C₆-alkynylamino, di(C₁-C₆-alkyl)amino, di(C₂-C₆-alkenyl)amino, di(C₂-C₆-alkynyl)amino, C₁-C₆-alkylthio, C₂-C₆-alkenylthio, C₂-C₆-alkynylthio, C₁-C₆-alkylsulfonyl, C₂-C₆-alkenylsulfonyl, C₂-C₆-alkynylsulfonyl, (C₁-C₆-alkyl)carbonyl, (C₂-C₆-alkenyl)-carbonyl, (C₂-C₆-alkynyl)-carbonyl, C₁-C₆-alkoxy, C₂-C₆-alkenyloxy, C₂-C₆-alkynyloxy, (C₁-C₆-alkoxy)carbonyl, (C₂-C₆-alkenyloxy)carbonyl, (C₂-C₆-alkynyloxy)-carbonyl, (C₁-C₆-alkyl)carbonyloxy, (C₂-C₆-alkenyl-)carbonyl-oxy, (C₂-C₆-alkynyl)carbonyloxy, (C₁-C₆-alkyl)carbonyl-amino, (C₂-C₆-alkenyl)carbonyl-amino, (C₂-C₆-alkynyl)carbonyl-amino,
   wherein the aliphatic parts of the aforementioned groups may be unsubstituted, partially or completely halogenated and/or may carry any combination of one, two or three radicals, independently of one another selected from the group consisting of CN, NO₂, OH, SH, NH₂, CO₂H, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-haloalkoxy, C₁-C₆-haloalkyl and C₁-C₆-alkylthio;
C(O)NR^{a}R^{b}, C(S)NR^{a}R^{b}, (SO₂)NR^{a}R^{b}, C(═O)R^{c} or C(═S)R^{c};
a radical Y—Ar or a radical Y-Cy, wherein
Y is a single bond, O, S, NH, C₁-C₆-alkandiyl or C₁-C₆-alkanyloxy,
Ar is phenyl, naphthyl or a mono- or bicyclic 5- to 10-membered heteroaromatic ring, which contains 1, 2, 3 or 4 heteroatoms selected from oxygen, sulfur and nitrogen atoms as ring members,
   wherein Ar is unsubstituted or may carry any combination of 1, 2, 3, 4 or 5 radicals, independently of one another selected from the group consisting of halogen, CN, NO₂, OH, SH, NH₂, CO₂H, C₁-C₆-alkyl, C₁-C₆-haloalkyl, C₁-C₆-alkoxy, C₂-C₆-alkenyloxy, C₂-C₆-alkynyloxy, C₁-C₆-haloalkoxy and C₁-C₆-alkylthio;
Cy is C₃-C₈-cycloalkyl, which is unsubstituted or may carry any combination of 1, 2, 3, 4 or 5 radicals, independently of one another selected from the group consisting of halogen, CN, NO₂, OH, SH, NH₂, CO₂H, C₁-C₆-alkyl, C₁-C₆-haloalkyl, C₁-C₆-alkoxy, C₂-C₆-alkenyloxy, C₂-C₆-alkynyloxy, C₁-C₆-haloalkoxy and C₁-C₆-alkylthio;
and wherein
R^{a} and R^{b} are each independently selected from one another from hydrogen, C₁-C₆-alkyl, C₁-C₆-haloalkyl, C₂-C₆-alkenyl, C₂-C₆-haloalkenyl, C₂-C₆-alkynyl, or C₂-C₆-haloalkynyl,
   wherein 1, 2 or 3 hydrogen atoms in the aforementioned aliphatic radicals may be replaced, independently of one another, by a radical selected from the group consisting of CN, NO₂, OH, SH, NH₂, CO₂H, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-haloalkoxy, C₁-C₆-haloalkyl and C₁-C₆-alkylthio;
R^{c} is selected from hydrogen, C₁-C₆ alkyl, C₂-C₆-alkenyl, C₂-C₆-alkynyl, C₃-C₈-cycloalkyl, C₁-C₆-alkylthio, C₁-C₆-alkoxy, (C₁-C₆-alkyl)amino, di(C₁-C₆-alkyl)amino, hydrazino, (C₁-C₆-alkyl)hydrazino, di(C₁-C₆-alkyl)hydrazino,
   wherein the aliphatic parts of the aforementioned groups may be unsubstituted, partially or completely halogenated or may carry any combination of one, two or three radicals, independently of one another selected from the group consisting of CN, NO₂, OH, SH, NH₂, CO₂H, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-haloalkoxy, C₁-C₆-haloalkyl and C₁-C₆-alkylthio;
phenyl or a mono- or bicyclic 5- to 10-membered heteroaromatic ring, which contains 1, 2, 3 or 4 heteroatoms selected from oxygen, sulfur and nitrogen atoms as ring members,
   wherein the phenyl or the heteroaromatic ring are unsubstituted or may carry any combination of 1, 2, 3, 4 or 5 radicals, independently of one another selected from the group consisting of halogen, CN, NO₂, OH, SH, NH₂, CO₂H, C₁-C₆-alkyl, C₁-C₆-haloalkyl, C₁-C₆-alkoxy, C₂-C₆-alkenyloxy, C₂-C₆-alkynyloxy, C₁-C₆-haloalkoxy and C₁-C₆-alkylthio;
      and the salts, enantiomers or diasteromers thereof.

Therefore, the present invention relates to 1-(azolin-2-yl)-amino-1,2-heterocyclyl-ethane compounds of the general formula I, the enantiomers, diasteromers and the salts thereof. These compounds have a high pesticidal activity and are active against a broad spectrum of animal pests selected from insects, arachnids and nematodes.

The compounds of formula I, the enantiomers, diasteromers and their salts are particularly useful for combating animal pests. The present invention provides the use of compounds of formula I, the enantiomers, diasteromers and the salts thereof for protecting plants against damage by animal pest.

Accordingly, the present invention also relates to a method for combating animal pests, in particular insects, arachnids and nematodes, by treating said pest with at least one compound of formula I, the enantiomers, diasteromers and/or salt thereof. The method comprises contacting the animal pests, or the environment in which the animal pests live or growing or may live or grow or the materials, plants, seeds, soils, surfaces or spaces to be protected from animal attack or infestation with at least one 1-(azolin-2-yl)-amino-1,2-heterocyclyl-ethane compound of the formula I, the enantiomers, diasteromers and/or salt thereof.

The present invention also provides a method for protecting crops from attack or infestation by animal pests, in particular insects, arachnids and nematodes. Said method comprises contacting a crop with at least one 1-(azolin-2-yl)-amino-1,2-heterocyclyl-ethane compound of formula I, the enantiomers, diasteromers and/or salt thereof.

Furthermore, the present invention provides a method for the protection of seeds from soil insects and of the seedlings' roots and shoots from insects. Said method comprises contacting the seeds before sowing and/or after pregermination with at least one 1-(azolin-2-yl)-amino-1,2-heterocyclyl-ethane compound of formula I, the enantiomers, diasteromers and/or salt thereof.

Furthermore, the invention relates to seed, comprising at least one 1-(azolin-2-yl)-amino-1,2-heterocyclyl-ethane compound of formula I, the enantiomers, diasteromers and/or salt thereof.

Accordingly, the invention further provides compositions for combating animal pests, comprising at least one 1-(azolin-2-yl)-amino-1,2-heterocyclyl-ethane compound of the general formula I, the enantiomers, diasteromers and/or salt thereof, and at least one carrier material. Thus, the present invention relates to agricultural compositions for combating such pests, in particular insects, nematodes or arachnids, preferably in the form of directly sprayable solutions, emulsions, pastes oil dispersions, powders, materials for scattering, dusts or in the form of granules, which comprise at least one 1-(azolin-2-yl)-amino-1,2-heterocyclyl-ethane compound of the general formula I, the enantiomers, diasteromers and/or an agriculturally useful salt thereof and at least one agriculturally acceptable carrier.

The present invention also provides the use of compounds of formula I, the enantiomers, diasteromers and the salts thereof for combating parasites in and on animals.

Accordingly, the present invention also relates to a method for protecting animals against infestation or infection by parasites which comprises administering to the animals a parasitically effective amount of at least one 1-(azolin-2-yl)-amino-1,2-heterocyclyl-ethane compound of formula I, the enantiomers, diasteromers and/or salt thereof to the animal in need thereof.

The present invention also relates to a method for treating animals infestated or infected by parasites which comprises administering to the animals a parasitically effective amount of at least one 1-(azolin-2-yl)-amino-1,2-heterocyclyl-ethane compound of formula I, the enantiomers, diasteromers and/or salt thereof to the animal in need thereof.

Accordingly, the present invention provides a veterinary composition comprising at least one 1-(azolin-2-yl)-amino-1,2-heterocyclyl-ethane compound of formula I, the enantiomers, diasteromers and/or a veterinary useful salt thereof and at least one veterinary acceptable carrier.

Furthermore, the invention provides 1-(aminothiocarbonylamino)-1,2-heterocyclyl-ethane compound of the general formula II

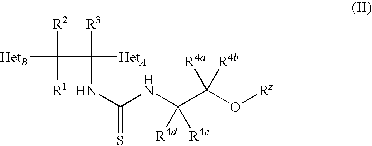

wherein Het_{A}, Het_{B}, R¹, R², R³, R^{4a}, R^{4b}, R^{4c} and R^{4d} have the meanings given above and wherein R^{z} is hydrogen, or acetyl, and the salts thereof. These compounds have a high pesticidal activity and are active against a broad spectrum of animal pests, in particular against insects, arachnids and nematodes. Therefore, the compounds of the formula II, the enantiomers, diasteromers and their salts can be used in similar methods and compositions as described for the compounds of the formula I .

The compounds of the formulae I and II may have one or more centers of chirality, in which case they are present as mixtures of stereoisomers, such as enantiomers or diastereomers. The present invention provides both the pure stereoisomers, e.g. the pure enantiomes or diastereomers, and mixtures thereof. The compounds of the formula I and II may also exist in the form of different tautomers. The invention comprises the single tautomers, if separable, as well as the tautomer mixtures. The scope of the present invention includes the (R)- and (S)-isomers and the racemates of compounds of the formulae I and II having chiral centers

Salts of the compounds of the formulae I and II are preferably agriculturally or veterinarily acceptable salts. They can be formed in a customary method, e.g. by reacting the compound with an acid of the anion in question if the compound of formulae I and II, respectively, has a basic functionality or by reacting an acidic compound of formulae I and II, respectively, with a suitable base.

Suitable agriculturally useful salts are especially the salts of those cations or the acid addition salts of those acids whose cations and anions, respectively, do not have any adverse effect on the action of the compounds according to the present invention. Suitable cations are in particular the ions of the alkali metals, preferably lithium, sodium and potassium, of the alkaline earth metals, preferably calcium, magnesium and barium, and of the transition metals, preferably manganese, copper, zinc and iron, and also ammonium (NH₄⁺) and substituted ammonium in which one to four of the hydrogen atoms are replaced by C₁-C₄-alkyl, C₁-C₄-hydroxyalkyl, C₁-C₄-alkoxy, C₁-C₄-alkoxy-C₁-C₄-alkyl, hydroxy-C₁-C₄-alkoxy-C₁-C₄-alkyl, phenyl or benzyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethylammonium, bis(2-hydroxyethyl)ammonium, benzyltrimethylammonium and benzyltriethylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogen sulfate, sulfate, dihydrogen phosphate, hydrogen phosphate, phosphate, nitrate, hydrogen carbonate, carbonate, hexafluorosilicate, hexafluorophosphate, benzoate, and the anions of C₁-C₄-alkanoic acids, preferably formate, acetate, propionate and butyrate. They can be formed by reacting a compound of formulae I with an acid of the corresponding anion, preferably of hydrochloric acid, hydrobromic acid, sulfuric acid, phosphoric acid or nitric acid.

The organic moieties mentioned in the above definitions of the variables are—like the term halogen—collective terms for individual listings of the individual group members. The prefix Cₙ-Cₘ indicates in each case the possible number of carbon atoms in the group.

The term halogen denotes in each case fluorine, bromine, chlorine or iodine, in particular fluorine, chlorine or bromine.

Examples of other meanings are:

The term “C₁-C₆-alkyl” as used herein and in the alkyl moieties of C₁-C₆-alkoxy, C₁-C₆-alkylamino, di(C₁-C₆-alkyl)amino, C₁-C₆-alkylthio, C₁-C₆-alkylsulfonyl, C₁-C₆-alkylsulfoxyl, C₁-C₆-alkylcarbonyl, C₁-C₆-alkoxycarbonyl, C₁-C₆-alkylthiocarbonyl, and C₁-C₆-alkylcarbonyloxy refer to a saturated straight-chain or branched hydrocarbon group having 1 to 6 carbon atoms, especially 1 to 4 carbon groups, for example methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl. C₁-C₄-alkyl means for example methyl, ethyl, propyl, 1-methylethyl, butyl, 1-methylpropyl, 2-methylpropyl or 1,1-dimethylethyl.

The term “C₁-C₆-haloalkyl” as used herein refers to a straight-chain or branched saturated alkyl group having 1 to 6 carbon atoms (as mentioned above), where some or all of the hydrogen atoms in these groups may be replaced by halogen atoms as mentioned above, for example C₁-C₄-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and the like.

The term “C₁-C₆-alkoxy” as used herein refers to a straight-chain or branched saturated alkyl group having 1 to 6 carbon atoms (as mentioned above) which is attached via an oxygen atom. Examples include C₁-C₆-alkoxy such as methoxy, ethoxy, OCH₂—C₂H₅, OCH(CH₃)₂, n-butoxy, OCH(CH₃)—C₂H₅, OCH₂—CH(CH₃)₂, OC(CH₃)₃, n-pentoxy, 1-methylbutoxy, 2-methylbutoxy, 3-methylbutoxy, 1,1-dimethylpropoxy, 1,2-dimethylpropoxy, 2,2-dimethyl-propoxy, 1-ethylpropoxy, n-hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy, 1-ethyl-2-methylpropoxy and the like.

The term “C₁-C₆-haloalkoxy” as used herein refers to a C₁-C₆-alkoxy group as mentioned above wherein the hydrogen atoms are partially or fully substituted by fluorine, chlorine, bromine and/or iodine, i.e., for example, C₁-C₆-haloalkoxy such as chloromethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, pentafluoroethoxy, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, 2,2,3,3,3-pentafluoropropoxy, heptafluoropropoxy, 1-(fluoromethyl)-2-fluoroethoxy, 1-(chloromethyl)-2-chloroethoxy, 1-(bromomethyl)-2-bromoethoxy, 4-fluorobutoxy, 4-chlorobutoxy, 4-bromobutoxy, nona-fluorobutoxy, 5-fluoro-1-pentoxy, 5-chloro-1-pentoxy, 5-bromo-1-pentoxy, 5-iodo-1-pentoxy, 5,5,5-trichloro-1-pentoxy, undecafluoropentoxy, 6-fluoro-1-hexoxy, 6-chloro-1-hexoxy, 6-bromo-1-hexoxy, 6-iodo-1-hexoxy, 6,6,6-trichloro-1-hexoxy or dodecafluoro-hexoxy, in particular chloromethoxy, fluoromethoxy, difluoromethoxy, trifluoromethoxy, 2-fluoroethoxy, 2-chloroethoxy or 2,2,2-trifluoroethoxy.

The term “C₁-C₆-alkoxy-C₁-C₆-alkyl” as used herein refers to C₁-C₆-alkyl wherein 1 carbon atom carries a C₁-C₆-alkoxy radical as mentioned above. Examples are CH₂—OCH₃, CH₂—OC₂H₅, n-propoxymethyl, CH₂—OCH(CH₃)₂, n-butoxymethyl, (1-methylpropoxy)methyl, (2-methylpropoxy)methyl, CH₂—OC(CH₃)₃, 2-(methoxy)ethyl, 2-(ethoxy)ethyl, 2-(n-propoxy)ethyl, 2-(1-methylethoxy)ethyl, 2-(n-butoxy)ethyl, 2-(1-methylpropoxy)ethyl, 2-(2-methylpropoxy)ethyl, 2-(1,1-dimethylethoxy)ethyl, 2-(methoxy)propyl, 2-(ethoxy)propyl, 2-(n-propoxy)propyl, 2-(1-methylethoxy)propyl, 2-(n-butoxy)propyl, 2-(1-methylpropoxy)propyl, 2-(2-methylpropoxy)propyl, 2-(1,1-dimethylethoxy)propyl, 3-(methoxy)propyl, 3-(ethoxy)propyl, 3-(n-propoxy)propyl, 3-(1-methylethoxy)propyl, 3-(n-butoxy)propyl, 3-(1-methylpropoxy)propyl, 3-(2-methylpropoxy)propyl, 3-(1,1-dimethylethoxy)propyl, 2-(methoxy)butyl, 2-(ethoxy)butyl, 2-(n-propoxy)butyl, 2-(1-methylethoxy)butyl, 2-(n-butoxy)butyl, 2-(1-methylpropoxy)butyl, 2-(2-methylpropoxy)butyl, 2-(1,1-dimethylethoxy)butyl, 3-(methoxy)butyl, 3-(ethoxy)butyl, 3-(n-propoxy)butyl, 3-(1-methylethoxy)butyl, 3-(n-butoxy)butyl, 3-(1-methylpropoxy)butyl, 3-(2-methylpropoxy)butyl, 3-(1,1-dimethylethoxy)butyl, 4-(methoxy) butyl, 4-(ethoxy) butyl, 4-(n-propoxy)butyl, 4-(1-methylethoxy)butyl, 4-(n-butoxy)butyl, 4-(1-methylpropoxy)butyl, 4-(2-methylpropoxy)butyl, 4-(1,1-dimethylethoxy)butyl and the like.

The term “(C₁-C₆-alkyl)carbonyl” as used herein refers to a straight-chain or branched saturated alkyl group having 1 to 6 carbon atoms (as mentioned above) bonded via the carbon atom of the carbonyl group at any bond in the alkyl group. Examples include C₁-C₆-alkylcarbonyl such CO—CH₃, CO—C₂H₅, n-propylcarbonyl, 1-methylethylcarbonyl, n-butylcarbonyl, 1-methylpropylcarbonyl, 2-methylpropylcarbonyl, 1,1-dimethylethylcarbonyl, n-pentylcarbonyl, 1-methylbutylcarbonyl, 2-methylbutylcarbonyl, 3-methylbutylcarbonyl, 1,1-dimethylpropylcarbonyl, 1,2-dimethylpropylcarbonyl, 2,2-dimethylpropylcarbonyl, 1-ethylpropylcarbonyl, n-hexylcarbonyl, 1-methylpentylcarbonyl, 2-methylpentylcarbonyl, 3-methylpentylcarbonyl, 4-methylpentylcarbonyl, 1,1-dimethylbutylcarbonyl, 1,2-dimethylbutylcarbonyl, 1,3-dimethylbutylcarbonyl, 2,2-dimethylbutylcarbonyl, 2,3-dimethylbutylcarbonyl, 3,3-dimethylbutylcarbonyl, 1-ethylbutylcarbonyl, 2-ethylbutylcarbonyl, 1,1,2-trimethylpropylcarbonyl, 1,2,2-trimethylpropylcarbonyl, 1-ethyl-1-methylpropylcarbonyl or 1-ethyl-2-methylpropylcarbonyl and the like.

The term “(C₁-C₆-alkoxy)carbonyl” as used herein refers to a straight-chain or branched alkoxy group (as mentioned above) having 1 to 6 carbon atoms attached via the carbon atom of the carbonyl group, for example CO—OCH₃, CO—OC₂H₅, COO—CH₂—C₂H₅, CO—OCH(CH₃)₂, n-butoxycarbonyl, CO—OCH(CH₃)—C₂H₅, CO—OCH₂—CH(CH₃)₂, CO—OC(CH₃)₃, n-pentoxycarbonyl, 1-methylbutoxycarbonyl, 2-methylbutoxycarbonyl, 3-methylbutoxycarbonyl, 2,2-dimethylpropoxycarbonyl, 1-ethylpropoxycarbonyl, n-hexoxycarbonyl, 1,1-dimethylpropoxycarbonyl, 1,2-dimethylpropoxycarbonyl, 1-methylpentoxycarbonyl, 2-methylpentoxycarbonyl, 3-methylpentoxycarbonyl, 4-methylpentoxycarbonyl, 1,1-dimethylbutoxycarbonyl, 1,2-dimethylbutoxycarbonyl, 1,3-dimethylbutoxycarbonyl, 2,2-dimethylbutoxycarbonyl, 2,3-dimethylbutoxycarbonyl, 3,3-dimethylbutoxycarbonyl, 1-ethylbutoxycarbonyl, 2-ethylbutoxycarbonyl, 1,1,2-trimethylpropoxycarbonyl, 1,2,2-trimethylpropoxycarbonyl, 1-ethyl-1-methylpropoxycarbonyl or 1-ethyl-2-methylpropoxycarbonyl.

The term “(C₁-C₆-alkyl)carbonyloxy” as used herein refers to a straight-chain or branched saturated alkyl group having 1 to 6 carbon atoms (as mentioned above) bonded via the carbon atom of the carbonyloxy group at any bond in the alkyl group, for example O—CO—CH₃, O—CO—C₂H₅, n-propylcarbonyloxy, 1-methylethylcarbonyloxy, n-butylcarbonyloxy, 1-methylpropylcarbonyloxy, 2-methylpropylcarbonyloxy, 1,1-dimethylethylcarbonyloxy, n-pentylcarbonyloxy, 1-methylbutylcarbonyloxy, 2-methylbutylcarbonyloxy, 3-methylbutylcarbonyloxy, 1,1-dimethylpropylcarbonyloxy or 1,2-dimethylpropylcarbonyloxy.

The term “C₁-C₆-alkylthio” (C₁-C₆-alkylsulfanyl: C₁-C₆-alkyl-S—)” as used herein refers to a straight-chain or branched saturated alkyl group having 1 to 6 carbon atoms (as mentioned above) which is attached via a sulfur atom, for example C₁-C₄-alkylthio such as methylthio, ethylthio, propylthio, 1-methylethylthio, butylthio, 1-methylpropylthio, 2-methylpropylthio, 1,1-dimethylethylthio, n-pentylthiocarbonyl, 1-methylbutylthio, 2-methylbutylthio, 3-methylbutylthio, 2,2-dimethylpropylthio, 1-ethylpropylthio, n-hexylthio, 1,1-dimethylpropylthio, 1,2-dimethylpropylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylhio, 2,2-dimethylbutylthio, 2,3-dimethylbutylthio, 3,3-dimethylbutylthio, 1-ethylbutlthio, 2-ethylbutylthio, 1,1,2-trimethylpropylthio, 1,2,2-trimethylpropylthio, 1-ethyl-1-methylpropylthio or 1-ethyl-2-methylpropylthio.

The term “(C₁-C₆-alkylthio)carbonyl” as used herein refers to a straight-chain or branched alkthio group (as mentioned above) having 1 to 6 carbon atoms attached via the carbon atom of the carbonyl group. Examples include CO—SCH₃, CO—SC₂H₅, CO—SCH₂—C₂H₅, CO—SCH(CH₃)₂, n-butylthiocarbonyl, CO—SCH(CH₃)—C₂H₅, CO—SCH₂—CH(CH₃)₂, CO—SC(CH₃)₃, n-pentylthiocarbonyl, 1-methylbutylthiocarbonyl, 2-methylbutylthiocarbonyl, 3-methyl butylthiocarbonyl, 2,2-dimethylpropylthiocarbonyl, 1-ethylpropylthiocarbonyl, n-hexylthiocarbonyl, 1,1-dimethylpropylthiocarbonyl, 1,2-dimethylpropylthiocarbonyl, 1-methylpentylthiocarbonyl, 2-methylpentylthiocarbonyl, 3-methylpentylthiocarbonyl, 4-methylpentylthiocarbonyl, 1,1-dimethylbutylthiocarbonyl, 1,2-dimethylbutylthiocarbonyl, 1,3-dimethylbutylhiocarbonyl, 2,2-dimethylbutylthiocarbonyl, 2,3-dimethylbutylthiocarbonyl, 3,3-dimethylbutylthiocarbonyl, 1-ethylbutlthioycarbonyl, 2-ethylbutylthiocarbonyl, 1,1,2-trimethylpropylthiocarbonyl, 1,2,2-trimethylpropylthiocarbonyl, 1-ethyl-1-methylpropylthiocarbonyl or 1-ethyl-2-methylpropylthiocarbonyl.

The term “C₁-C₆-alkylsulfinyl” (C₁-C₆-alkylsulfoxyl: C₁-C₆-alkyl-S(═O)—), as used herein refers to a straight-chain or branched saturated alkyl group (as mentioned above) having 1 to 6 carbon atoms bonded through the sulfur atom of the sulfinyl group at any position in the alkyl group, for example SO—CH₃, SO—C₂H₅, n-propylsulfinyl, 1-methylethylsulfinyl, n-butylsulfinyl, 1-methylpropylsulfinyl, 2-methylpropylsulfinyl, 1,1-dimethylethylsulfinyl, n-pentylsulfinyl, 1-methylbutylsulfinyl, 2-methylbutylsulfinyl, 3-methylbutylsulfinyl, 1,1-dimethylpropylsulfinyl, 1,2-dimethylpropylsulfinyl, 2,2-dimethylpropylsulfinyl, 1-ethylpropylsulfinyl, n-hexylsulfinyl, 1-methylpentylsulfinyl, 2-methylpentylsulfinyl, 3-methylpentylsulfinyl, 4-methylpentylsulfinyl, 1,1-dimethylbutylsulfinyl, 1,2-dimethylbutylsulfinyl, 1,3-dimethylbutylsulfinyl, 2,2-dimethylbutylsulfinyl, 2,3-dimethylbutylsulfinyl, 3,3-dimethylbutylsulfinyl, 1-ethylbutylsulfinyl, 2-ethylbutylsulfinyl, 1,1,2-trimethylpropylsulfinyl, 1,2,2-trimethylpropylsulfinyl, 1-ethyl-1-methylpropylsulfinyl or 1-ethyl-2-methylpropylsulfinyl.

The term “C₁-C₆-alkylamino” refers to a secondary amino group carrying one alkyl group as defined above, e.g. methylamino, ethylamino, propylamino, 1-methylethylamino, butylamino, 1-methylpropylamino, 2-methylpropylamino, 1,1-dimethylethylamino, pentylamino, 1-methylbutylamino, 2-methylbutylamino, 3-methylbutylamino, 2,2-dimethylpropylamino, 1-ethylpropylamino, hexylamino, 1,1-dimethylpropylamino, 1,2-dimethylpropylamino, 1-methylpentylamino, 2-methylpentylamino, 3-methylpentylamino, 4-methylpentylamino, 1,1-dimethylbutylamino, 1,2-dimethylbutylamino, 1,3-dimethylbutylamino, 2,2-dimethylbutylamino, 2,3-dimethylbutylamino, 3,3-dimethylbutylamino, 1-ethylbutylamino, 2-ethylbutylamino, 1,1,2-trimethylpropylamino, 1,2,2-trimethylpropylamino, 1-ethyl-1-methylpropylamino or 1-ethyl-2-methylpropylamino.

The term “di(C₁-C₆-alkyl)amino)” refers to a tertiary amino group carrying two alkyl radicals as defined above, e.g. dimethylamino, diethylamino, di-n-propylamino, diisopropylamino, N-ethyl-N-methylamino, N-(n-propyl)-N-methylamino, N-(isopropyl)-N-methylamino, N-(n-butyl)-N-methylamino, N-(n-pentyl)-N-methylamino, N-(2-butyl)-N-methylamino, N-(isobutyl)-N-methylamino, N-(n-pentyl)-N-methylamino, N-(n-propyl)-N-ethylamino, N-(isopropyl)-N-ethylamino, N-(n-butyl)-N-ethylamino, N-(n-pentyl)-N-ethylamino, N-(2-butyl)-N-ethylamino, N-(isobutyl)-N-ethylamino or N-(n-pentyl)-N-ethylamino.

The term “C₁-C₆-alkylsulfonyl” (C₁-C₆-alkyl-S(═O)₂—) as used herein refers to a straight-chain or branched saturated alkyl group having 1 to 6 carbon atoms (as mentioned above) which is bonded via the sulfur atom of the sulfonyl group at any position in the alkyl group, for example SO₂—CH₃, SO₂—C₂H₅, n-propylsulfonyl, SO₂—CH(CH₃)₂, n-butylsulfonyl, 1-methylpropylsulfonyl, 2-methylpropylsulfonyl, SO₂—C(CH₃)₃, n-pentylsulfonyl, 1-methylbutylsulfonyl, 2-methylbutylsulfonyl, 3-methylbutylsulfonyl, 1,1-dimethylpropylsulfonyl, 1,2-dimethylpropylsulfonyl, 2,2-dimethylpropylsulfonyl, 1-ethylpropylsulfonyl, n-hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropylsulfonyl or 1-ethyl-2-methylpropylsulfonyl.

The term “C₂-C₆-alkenyl” as used herein and in the alkenyl moieties of C₂-C₆-alkenyloxy, C₂-C₆-alkenylamino, C₂-C₆-alkenylthio, C₂-C₆-alkenylsulfonyl, (C₂-C₆-alkenyl)carbonyl, (C₂-C₆-alkenyloxy)carbonyl and (C₂-C₆-alkenyl)carbonyloxy refers to a straight-chain or branched unsaturated hydrocarbon group having 2 to 6 carbon atoms and a double bond in any position, such as ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl; 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl.

The term, “C₂-C₆-alkenyloxy” as used herein refers to a straight-chain or branched alkenyl group having 2 to 6 carbon atoms (as mentioned above) which is attached via an oxygen atom, such as vinyloxy, allyloxy (propen-3-yloxy), methallyloxy, buten-4-yloxy, etc.

The term “C₂-C₆-alkenylthio” as used herein refers to a straight-chain or branched alkenyl group having 2 to 6 carbon atoms (as mentioned above) which is attached via a sulfur atom, for example vinylsulfanyl, allylsulfanyl (propen-3-ylthio), methallylsufanyl, buten-4-ylsulfanyl, etc.

The term “C₂-C₆-alkenylamino” as used herein refers to a straight-chain or branched alkenyl group having 2 to 6 carbon atoms (as mentioned above) which is attached via a sulfur atom, for example vinylamino, allylamino (propen-3-ylamino), methallylamino, buten-4-ylamino, etc.

The term “C₂-C₆-alkenylsulfonyl” as used herein refers to a straight-chain or branched alkenyl group having 2 to 6 carbon atoms (as mentioned above) which is attached via a sulfonyl (SO₂) group, for example vinylsulfonyl, allylsulfonyl (propen-3-ylsulfonyl), methallylsulfonyl, buten-4-ylsulfonyl, etc.

The term “C₂-C₆-alkynyl” as used herein and in the alkynyl moieties of C₂-C₆-alkynyloxy, C₂-C₆-alkynylamino, C₂-C₆-alkynylthio, C₂-C₆-alkynylsulfonyl, C₂-C₆-alkynylcarbonyl, C₂-C₆-alkynyloxycarbonyl and C₁-C₆-alkynylcarbonyloxy refers to a straight-chain or branched unsaturated hydrocarbon group having 2 to 6 carbon atoms and containing at least one triple bond, such as ethynyl, prop-1-yn-1-yl, prop-2-yn-1-yl, n-but-1-yn-1-yl, n-but-1-yn-3-yl, n-but-1-yn-4-yl, n-but-2-yn-1-yl, n-pent-1-yn-1-yl, n-pent-1-yn-3-yl, n-pent-1-yn-4-yl, n-pent-1-yn-5-yl, n-pent-2-yn-1-yl, n-pent-2-yn-4-yl, n-pent-2-yn-5-yl, 3-methylbut-1-yn-3-yl, 3-methylbut-1-yn-4-yl, n-hex-1-yn-1-yl, n-hex-1-yn-3-yl, n-hex-1-yn-4-yl, n-hex-1-yn-5-yl, n-hex-1-yn-6-yl, n-hex-2-yn-1-yl, n-hex-2-yn-4-yl, n-hex-2-yn-5-yl, n-hex-2-yn-6-yl, n-hex-3-yn-1-yl, n-hex-3-yn-2-yl, 3-methylpent-1-yn-1-yl, 3-methylpent-1-yn-3-yl, 3-methylpent-1-yn-4-yl, 3-methylpent-1-yn-5-yl, 4-methylpent-1-yn-1-yl, 4-methylpent-2-yn-4-yl or 4-methylpent-2-yn-5-yl and the like.

The term, “C₂-C₆-alkynyloxy” as used herein refers to a straight-chain or branched alkynyl group having 2 to 6 carbon atoms (as mentioned above) which is attached via an oxygen atom, such as propargyloxy (propyn-3-yloxy), butyn-3-yloxy, and butyn-4-yloxy.

The term “C₂-C₆-alkynylthio” as used herein refers to a straight-chain or branched alkynyl group having 2 to 6 carbon atoms (as mentioned above) which is attached via a sulfur atom, such as propargylsulfanyl (propyn-3-ylthio), butyn-3-ylsufanyl and butyn-4-ylsulfanyl.

The term “C₂-C₆-alkynylamino” as used herein refers to a straight-chain or branched alkynyl group having 2 to 6 carbon atoms (as mentioned above) which is attached via a sulfur atom, such as propargylamino (propyn-3-ylamino), butyn-3-amino, and butyn-4-ylamino.

The term “C₂-C₆-alkynylsulfonyl” as used herein refers to a straight-chain or branched alkynyl group having 2 to 6 carbon atoms (as mentioned above) which is attached via a sulfonyl (SO₂) group, such as propargylsulfonyl (propin-3-yltsulfonyl), butin-3-ylsulfonyl and butin-4-ylsulfonyl.

The terms “C₃-C₆-cycloalkyl”, “C₃-C₈-cycloalkyl” or “C₃-C₁₀-cycloalkyl” as used herein refer to a mono- or bi- or polycyclic hydrocarbon radical having 3 to 6, 3 to 8 or 3 to 10 carbon atoms respectively, in particular 3, 4, 5 and 6 carbon atoms. Examples of monocyclic radicals comprise cyclopropyl, cyclobutyl, cyclopentyl, cyclohexyl, cyclo-heptyl, cyclooctyl, cyclononyl and cyclodecyl. Examples of bicyclic radicals comprise bicyclo[2.2.1]heptyl, bicyclo[3.1.1]heptyl, bicyclo[2.2.2]octyl and bicyclo[3.2.1]octyl.

The term “C₃-C₆-halocycloalkyl” as used herein refers to a mono- or bi- or polycyclic hydrocarbon radical having 3 to 6 carbon atoms respectively, in particular 3, 4, 5 and 6 carbon atoms, wherein some or all of the hydrogen atoms in these groups may be replaced by halogen atoms. Examples of halocycloalkyl radicals comprise 3-membered halogenated cycloalkyls as 1-chlorocyclopropyl, 1-bromocyclopropyl, 1-fluorocyclopropyl, 2-chlorocyclopropyl, 2-bromocyclopropyl, 2-fluorocyclopropyl, 2,2-dichlorocyclopropyl, 2,2-dibromocyclopropyl, 2,2-difluorocyclopropyl, 1,2,2-trichlorocyclopropyl, 1,2,2-tribromocyclopropyl, 1,2,2-trifluorocyclopropyl, 2-chloro-2-fluorocyclopropyl, 1-chloro-2,2-difluorocyclopropyl, 1,2-dichloro-2-fluorocyclopropyl, 2,2,3-trichlorocyclopropyl, pentafluorocyclopropyl and the like; 4-membered halogenated cycloalkyls as 1-chlorocyclobutyl, 1-bromocyclobutyl, 1-fluorocyclobutyl, 2-chlorocyclobutyl, 2-bromocyclobutyl, 2-fluorocyclobutyl, 3-chlorocyclobutyl, 3-bromocyclobutyl, 3-fluorocyclobutyl, 2,2-dichlorocyclobutyl, 2,2-dibromocyclobutyl, 2,2-difluorocyclobutyl, 3,3-dichlorocyclobutyl, 3,3-dibromocyclobutyl, 3,3-difluorocyclobutyl, 1,2-dichlorocyclobutyl, 1,2-dibromocyclobutyl, 1,2-difluorocyclobutyl, 1,2,2-trichlorocyclobutyl, 1,2,2-tribromocyclobutyl, 1,2,2-trifluorocyclobutyl and the like; 5-membered halogenated cycloalkyls as 1-chlorocyclopentyl, 1-bromocyclopentyl, 1-fluorocyclopentyl, 2-chlorocyclopentyl, 2-bromocyclopentyl, 2-fluorocyclopentyl, 3-chlorocyclopentyl, 3-bromocyclopentyl, 3-fluorocyclopentyl, 2,2-dichlorocyclopentyl, 2,2-dibromocyclopentyl, 2,2-difluorocyclopentyl, 3,3-dichlorocyclopentyl, 3,3-dibromocyclopentyl, 3,3-difluorocyclopentyl, 1,2-dichlorocyclopentyl, 1,2-dibromocyclopentyl, 1,2-difluorocyclopentyl, 1,2,2-trichlorocyclopentyl, 1,2,2-tribromocyclopentyl, 1,2,2-trifluorocyclopentyl and the like and 6-membered halogenated cycloalkyls as 1-chlorocyclohexyl, 1-bromocyclohexyl, 1-fluorocyclohexyl, 2-chlorocyclohexyl, 2-bromocyclohexyl, 2-fluorocyclohexyl, 3-chlorocyclohexyl, 3-bromocyclohexyl, 3-fluorocyclohexyl, 2,2-dichlorocyclohexyl, 2,2-dibromocyclohexyl, 2,2-difluorocyclohexyl, 3,3-dichlorocyclohexyl, 3,3-dibromocyclohexyl, 3,3-difluorocyclohexyl, 4,4-dichlorocyclohexyl, 4,4-dibromocyclohexyl, 4,4-difluorocyclohexyl, 1,2-dichlorocyclohexyl, 1,2-dibromocyclohexyl, 1,2-difluorocyclohexyl, 1,3-dichlorocyclohexyl, 1,3-dibromocyclohexyl, 1,3-difluorocyclohexyl, 1,4-dichlorocyclohexyl, 1,4-dibromocyclohexyl, 1,4-difluorocyclohexyl, 1,2,2-trichlorocyclohexyl, 1,2,2-tribromocyclohexyl, 1,2,2-trifluorocyclohexyl and the like.

The term “5- or 6-membered aromatic heterocycle” as used herein refers to a monocyclic heteroaromatic radical which has 5 or 6 ring members, which may comprise a fused 3, 4, 5, 6 or 7 membered ring thus having a total number of ring members from 8 to 10, wherein in each case 1, 2, 3 or 4 of these ring members are heteroatoms selected, independently from each other, from the group consisting of oxygen, nitrogen and sulfur. The heterocyclic radical may be attached to the remainder of the molecule via a carbon ring member or via a nitrogen ring member. The fused ring includes e.g. C₅-C₇-cycloalkyl, C₅-C₇-cycloalkenyl, or 5 to 7 membered heterocyclyl and phenyl.

Examples for monocyclic 5- to 6-membered heteroaromatic rings include triazinyl, pyrazinyl, pyrimidyl, pyridazinyl, pyridyl, thienyl, furyl, pyrrolyl, pyrazolyl, imidazolyl, triazolyl, tetrazolyl, thiazolyl, oxazolyl, thiadiazolyl, oxadiazolyl, isothiazolyl and isoxazolyl.

Examples for 5- to 6-membered heteroaromatic rings carrying a fused phenyl ring are quinolinyl, isoquinolinyl, indolyl, indolizinyl, isoindolyl, indazolyl, benzofuryl, benzthienyl, benzo[b]thiazolyl, benzoxazolyl, benzthiazolyl, benzoxazolyl, and benzimidazolyl. Examples for 5- to 6-membered heteroaromatic rings carrying a fused cycloalkenyl ring are dihydroindolyl, dihydroindolizinyl, dihydroisoindolyl, dihydrochinolinyl, dihydroiso-chinolinyl, chromenyl, chromanyl and the like.

The term “mono- or bicyclic” “5- to 10-membered” or “5, 6 or 7-membered” “saturated or partially unsaturated” “heterocyclic ring or heterocycle” comprises monocyclic and bicyclic nonaromatic saturated or partially unsaturated heterocyclic rings having 5, 6, 7, 8, 9 or 10 ring members. Examples for non-aromatic rings include pyrrolidinyl, pyrazolinyl, imidazolinyl, pyrrolinyl, pyrazolinyl, imidazolinyl, tetrahydrofuranyl, dihydrofuranyl, 1,3-dioxolanyl, dioxolenyl, thiolanyl, dihydrothienyl, oxazolidinyl, isoxazolidinyl, oxazolinyl, isoxazolinyl, thiazolinyl, isothiazolinyl, thiazolidinyl, isothiazolidinyl, oxathiolanyl, piperidinyl, piperazinyl, pyranyl, dihydropyranyl, tetrahydropyranyl, dioxanyl, thiopyranyl, dihydrothiopyranyl, tetrahydrothiopyranyl, morpholinyl, thiazinyl and the like.

The term “5-, 6- or 7-membered carbocycle” comprises monocyclic aromatic rings and nonaromatic saturated or partially unsaturated carbocyclic rings having 5, 6 or 7 ring members. Examples for non-aromatic rings include cyclopentyl, cyclopentenyl, cyclopentadienyl, cyclohexyl, cyclohexenyl, cyclohexadienyl, cycloheptyl, cycloheptenyl, cycloheptadienyl and the like.

As regards the pesticidal activity of the compounds of general formula I, preference is given to those compounds of the formula I, wherein the variables m, X, R¹, R², R³, R^{4a}, R^{4b}, R^{4c}, R^{4d}, R⁵, R⁶, R⁷, R⁸, R⁹, R^{a}, R^{b}, R^{c}, Het_{A} and Het_{B} have independently of each other or more preferably in combination the following meanings:

Preferred are compounds of the formula I, wherein R¹, R², R³ are, independently of each other, selected from hydrogen, halogen, C₁-C₆-alkyl, C₁-C₆-haloalkyl, C₃-C₆-cycloalkyl and C₃-C₆-halocycloalkyl,
wherein 1, 2 or 3 hydrogen atoms in the aforementioned aliphatic radicals may be replaced, independently of one another, by a radical selected from the group consisting of CN, NO₂, OH, SH, NH₂, CO₂H, C₂-C₆-alkenyl, C₂-C₆-alkynyl, C₁-C₆-alkoxy, C₂-C₆-alkenyloxy, C₂-C₆-alkynyloxy, C₁-C₆-haloalkoxy and C₁-C₆-alkylthio, and wherein C₃-C₆-cycloalkyl and C₃-C₆-halocycloalkyl may also carry 1, 2, 3, 4 or 5 radicals selected from C₁-C₆-alkyl and C₁-C₆-haloalkyl.

More preferred are compounds of the formula I, wherein R¹, R² and R³ are, independently of each other, selected from hydrogen, halogen and C₁-C₆-alkyl, especially methyl, ethyl, n-propyl, iso-propyl, n-butyl and isobutyl.

Especially preferred are compounds of the formula I, wherein R¹, R² and R³ are, independently of each other, selected from hydrogen and C₁-C₆-alkyl

Most preferred are compounds of the formula I, wherein R¹, R² and R³ are hydrogen. Especially preferred are compounds of the formula I, wherein R³ is hydrogen.

Preference is given to compounds of the formula I wherein R^{4a}, R^{4b}, R^{4c} and R^{4d} are, independently of each other, selected from hydrogen, halogen, C₁-C₆-alkyl, especially methyl or ethyl, and C₁-C₆-haloalkyl.

More preference is given to compounds of the formula I wherein R^{4a}, R^{4b}, R^{4c} and R^{4d} are selected from hydrogen.

Likewise, preference is given to compounds I, wherein one of the radicals R^{4a}, R^{4b}, R^{4c} or R^{4d} is selected from halogen, C₁-C₆-alkyl, especially methyl or ethyl, and C₁-C₆-haloalkyl and the other radicals R^{4a}, R^{4b}, R^{4c} or R^{4d} are hydrogen.

Preference is furthermore given to compounds of the formula I in which R⁵ or R⁶ is selected from hydrogen, CN, NO₂, C₁-C₆-alkyl, C₂-C₆-alkenyl, C₂-C₆-alkynyl, C₃-C₈-cycloalkyl, (C₁-C₆-alkoxy)methylen, C₁-C₆-alkylsulfanyl, C₁-C₆-alkylsulfinyl and C₁-C₆-alkylsulfonyl, C(O)NR^{a}R^{b}, C(S)NR^{a}R^{b}, C(═O)R^{c} and C(═S)R^{c},
wherein R^{a}, R^{b} and R^{c} are as defined above, and
wherein the aliphatic moieties in the aforementioned radicals may be unsubstituted, partially or completely halogenated and/or may carry 1, 2 or 3 radicals, which are independently of one another, selected from the group consisting of CN, NO₂, OH, SH, NH₂, CO₂H, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-haloalkoxy and C₁-C₆-alkylthio and
wherein C₃-C₈-cycloalkyl may also carry 1, 2, 3, 4 or 5 radicals selected from C₁-C₆-alkyl and C₁-C₆-haloalkyl.

Amongst these, particular preference is given to those compounds I wherein R⁵ or R⁶ is selected from hydrogen, CN, NO₂, C₁-C₆-alkyl and C(═O)R^{c}, wherein R^{c} is as defined above.

If present, R^{c} is preferably selected from hydrogen, C₁-C₆ alkyl, C₂-C₆-alkenyl, C₂-C₆-alkynyl, C₃-C₈-cycloalkyl, C₁-C₆-alkylthio, C₁-C₆-alkoxy, (C₁-C₆-alkyl)amino, di(C₁-C₆-alkyl)amino, hydrazino, (C₁-C₆-alkyl)hydrazino, di(C₁-C₆-alkyl)hydrazino, phenyl or a mono- or bicyclic 5- to 10-membered heteroaromatic ring, which contains 1, 2, 3 or 4 heteroatoms selected from O, S and N.

Preference is given to compounds I wherein X is sulfur.

Likewise, preference is given to compounds I wherein X is oxygen.

Likewise, preference is also given to compounds I in wherein X is NR⁷ and wherein R⁷ is as defined above.

Likewise, preference is also given to compounds I in wherein X is NR⁷ and wherein R⁷ is more preferably defined as below.

Preferably R⁷ is selected from hydrogen, CN, NO₂, C(═O)—R^{c}, especially formyl, C₁-C₆-alkylcarbonyl or benzoyl, C₁-C₆-alkyl, C₂-C₆-alkenyl, C₂-C₆-alkynyl, C₃-C₈-cycloalkyl, (C₁-C₆-alkoxy)methylen, C₁-C₆-alkylsulfanyl, C₁-C₆-alkylsulfinyl or C₁-C₆-alkylsulfonyl,
wherein the aliphatic moieties in the aforementioned radicals may be unsubstituted, partially or completely halogenated and/or may carry 1, 2 or 3 radicals, which are independently of one another, selected from the group consisting of CN, NO₂, OH, SH, NH₂, CO₂H, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-haloalkoxy and C₁-C₆-alkylthio and
wherein C₃-C₈-cycloalkyl may also carry 1, 2, 3, 4 or 5 radicals selected from C₁-C₆-alkyl and C₁-C₆-haloalkyl.

In particular R⁷ is selected from hydrogen, CN, NO₂, C(═O)R^{c}, especially benzoyl, formyl or C₁-C₆-alkylcarbonyl such as acetyl or ethylcarbonyl, C₁-C₆-alkyl, especially methyl, ethyl, n-propyl, isopropyl, n-butyl, isobutyl, n-pentyl, n-hexyl, with hydrogen, C₁-C₆-alkyl or a radical C(═O)R^{c} wherein R^{c} is H, C₁-C₆-alkyl or phenyl, being most preferred.

Preference is given to compounds I wherein the carbon atom which carries the radical A has S-configuration.

Preference is also given to compounds I wherein the carbon atom which carries the radical A has R-configuration.

Het_{A} is preferably a C-bound 5- or 6-membered, in particular a 5-membered C-bound heteroaromatic ring as defined above which is unsubstituted or substituted by m radicals R⁸ and/or may carry at its nitrogen atom, if present, a radical R⁹ or oxygen, with m being 0, 1, 2 or 3, in particular 0, 1 or 2.

Het_{B} is preferably a C-bound 5- or 6-membered heteroaromatic ring, in particular a 5-membered C-bound heteroaromatic ring as defined above which is unsubstituted or substituted by m radicals R⁸ and/or may carry at its nitrogen atom, if present, a radical R⁹ or oxygen, with m being 0, 1, 2 or 3, in particular 0, 1 or 2.

In another preferred embodiment, Het_{B} is bound via the heteroatom of a 5- or 6-membered heteroaromatic ring, in particular a 5- or 6-membered N-bound heteroaromatic ring comprising 1, 2 or 3 nitrogen atoms as heteroatom(s) and further defined as above, which is unsubstituted or substituted by m radicals R⁸ and/or may carry at its nitrogen atom, if present, a radical R⁹ or oxygen, with m being 0, 1, 2 or 3, in particular 0, 1 or 2.

A preferred embodiment of the invention relates to compounds of the formula I in which the integer m is different from 0.

A preferred embodiment of the invention relating to compounds of the formula I in which the m is different from 0 means that m is 1, 2 or 3.

A more preferred embodiment of the invention relating to compounds of the formula I in which the m is different from 0, means that m is 1 or 2.

If present, R⁸ is preferably selected from halogen, OH, SH, NH₂, SO₃H, COOH, CN, CONH₂, C(═O)R^{c}, C₁-C₆-alkyl, C₃-C₈-cycloalkyl, C₁-C₆-alkylamino, di(C₁-C₆-alkyl)amino,
wherein the aliphatic moieties in the aforementioned radicals may be unsubstituted, partially or completely halogenated and/or may carry 1, 2 or 3 radicals, which are independently of one another, selected from the group consisting of cyano, nitro, hydroxy, mercapto, amino, carboxyl, C₂-C₆-alkenyl, C₂-C₆-alkynyl, C₁-C₆-alkoxy, C₂-C₆-alkenyloxy, C₂-C₆-alkynyloxy, C₁-C₆-haloalkoxy and C₁-C₆-alkylthio and
wherein C₃-C₈-cycloalkyl may also carry 1, 2, 3, 4 or 5 radicals selected from C₁-C₆-alkyl and C₁-C₆-haloalkyl.

More preferably, R⁸ is selected from halogen, especially chlorine, C₁-C₆-alkyl, in particular C₁-C₄-alkyl, such as methyl, ethyl, n-propyl, isopropyl, n-butyl, isobutyl or tert-butyl, C₁-C₆-haloalkyl, in particular C₁-C₄-haloalkyl, especially C₁-C₂-fluoroalkyl such as trifluoromethyl, difluoromethyl or 2,2,2-trifluoroethyl, C₁-C₆-alkoxy, especially methoxy, ethoxy or propoxy, and C₁-C₆-haloalkoxy, especially C₁-C₂-fluoroalalkyl such as trifluoromethoxy or difluoromethoxy.

If present, R⁹ is preferably hydrogen or C₁-C₆-alkyl, such as methyl, ethyl, n-propyl, isopropyl, n-butyl, isobutyl or tert.-butyl, more preferably hydrogen or C₁-C₄-alkyl, in particular hydrogen, methyl or ethyl.

In particular, Het_{A} and/or Het_{B} are unsubstituted or carry independently from each other 1 or 2 radicals R⁸. Likewise preference is given to compounds I, wherein Het_{A} and/or Het_{B} are unsubstituted or substituted independently from each other by 1 or 2 radicals R⁸ and 1 radical R⁹.

Preference is given to compounds I wherein Het_{A} and/or Het_{B} is a 5-membered heteroaromatic ring, in particular a 5-membered, C-bound heteroaromatic ring, as defined above, with Het_{A} and/or Het_{B} being unsubstituted or substituted by m radicals R⁸ and/or may carrying at its nitrogen atom, if present, a radical R⁹ as defined above. Particular preference is given to those 5-membered heteroaromatic radicals Het_{A} and or Het_{B} which are selected from 2-furyl, 3-furyl, 2-thienyl, 3-thienyl, 2-pyrrolyl, 3-pyrolyl, 2-oxazolyl, 4-oxazolyl, 5-oxazolyl, 2-thiazoyl, 4-thiazolyl, 5-thiazolyl, 2-imidazolyl, 4-imidazolyl, 5-imidazolyl, 3-isoxazolyl, 4-isoxazolyl, 5-isoxazoly, 3-isothiazolyl, 4-isothiazolyl, 5-isothiazoly, 3-pyrazolyl, 4-pyrazolyl, 5-pyrazolyl, 1,3,4-oxadiazol-2-yl, 1,3,4-thiadiazol-2-yl, 1,2,4-oxadiazol-3-yl, 1,2,4-thiadiazol-5-yl, 1,2,4-oxadiazol-5-yl, 1,2,4-thiadiazol-5-yl, 4H-1,2,4-triazol-3-yl, 1H-1,2,4-triazol-3-yl, 2H-1,2,4-triazol-3-yl, 3H-1,2,3-triazol-4-yl, 1H-1,2,3-triazol-4-yl, 1H-1,2,3,4-tetrazol-5-yl, 1,2,3-oxadiazol-4-yl, 1,2,3-oxadiazol-5-yl, 1,2,3-thiadiazol-4-yl and 1,2,3-thiadiazol-5-yl. Het_{A} and/or Het_{B} may be unsubstituted or substituted by m radicals R⁸ and/or may carry at its nitrogen atom, if present, a radical R⁹ wherein R⁸, R⁹ and m are as defined above.

Preference is also given to compounds of formula I wherein Het_{A} and/or Het_{B} are a 6-membered heteroaromatic ring as defined above. Particular preference is given to those 6-membered heteroaromatic radicals Het_{A} and/or Het_{B} which are selected from pyridin-2-yl, N-oxide of pyridin-2-yl, pyridin-3-yl, N-oxide of pyridin-3-yl, pyridin-4-yl, N-oxide of pyridin-4-yl, pyridazin-3-yl, pyridazin-4-yl, pyrimidin-2-yl, pyrimidin-4-yl, pyrimidin-5-yl, pyrazin-2-yl, 1,3,5-triazin-2-yl, 1,2,4-triazin-3-yl, 1,2,4-triazin-5-yl, 1,2,4-triazin-6-yl and 1,2,4,5-tetrazin-3-yl and wherein Het_{A} and/or Het_{B} is unsubstituted or substituted by m radicals R⁸. If present, R⁸ has the meanings given above, especially those meanings given as being preferred.

Particular preferences are given to compounds of formula I, wherein Het_{A} and/or Het_{B} are selected independently from one another from the radicals of the formulae Het.1 to Het.63 as defined below:

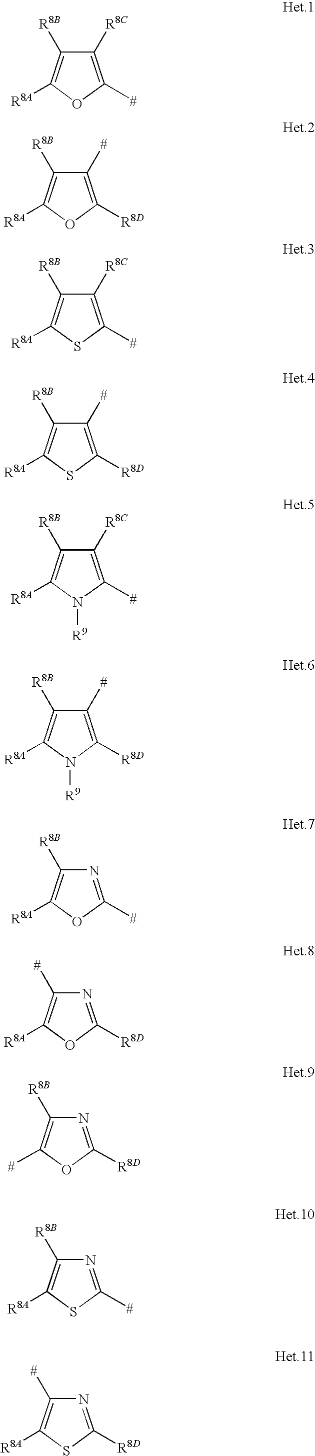

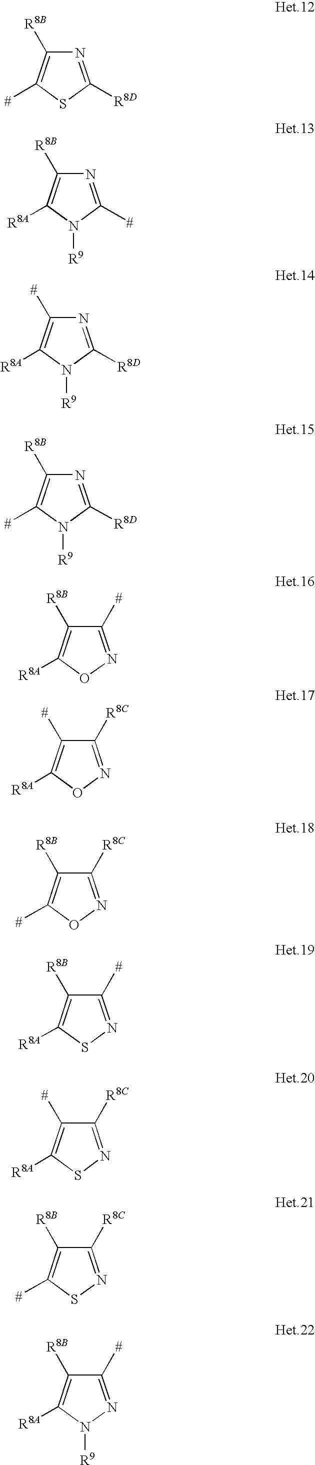

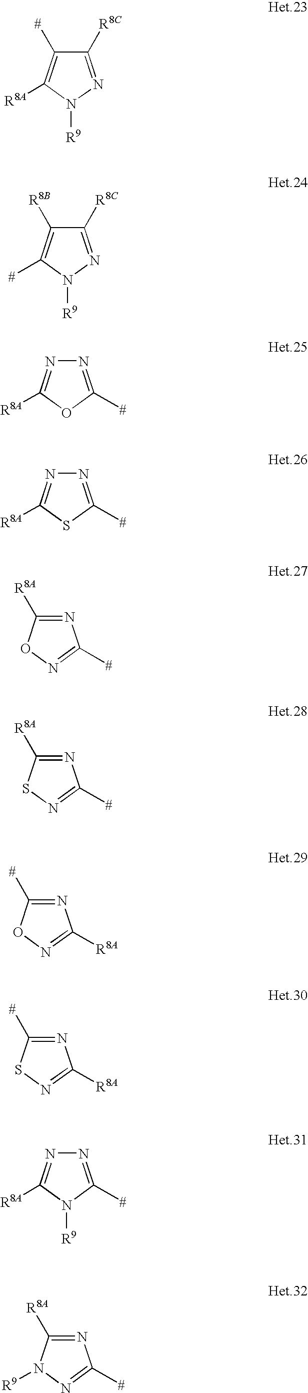

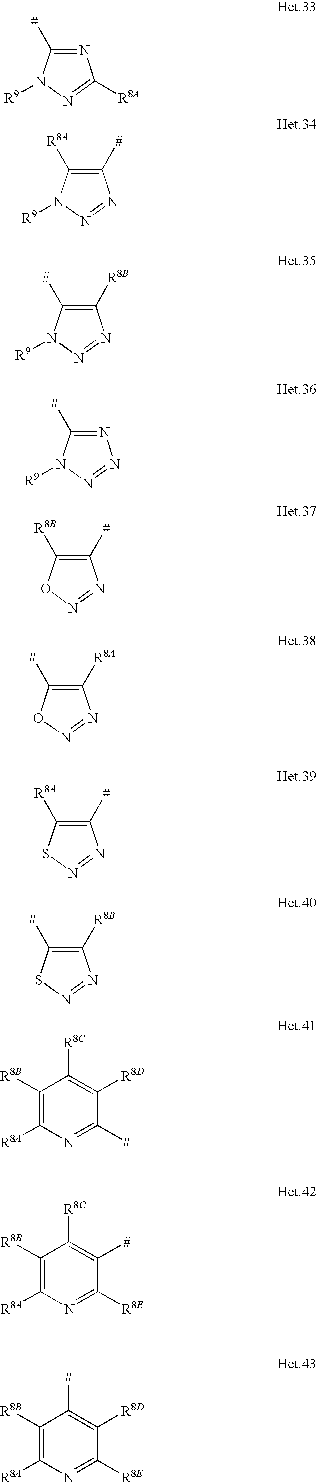

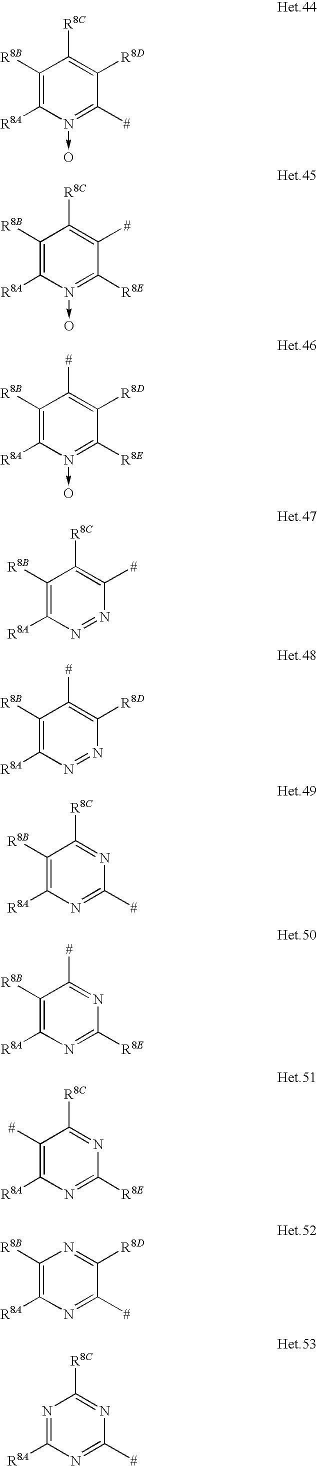

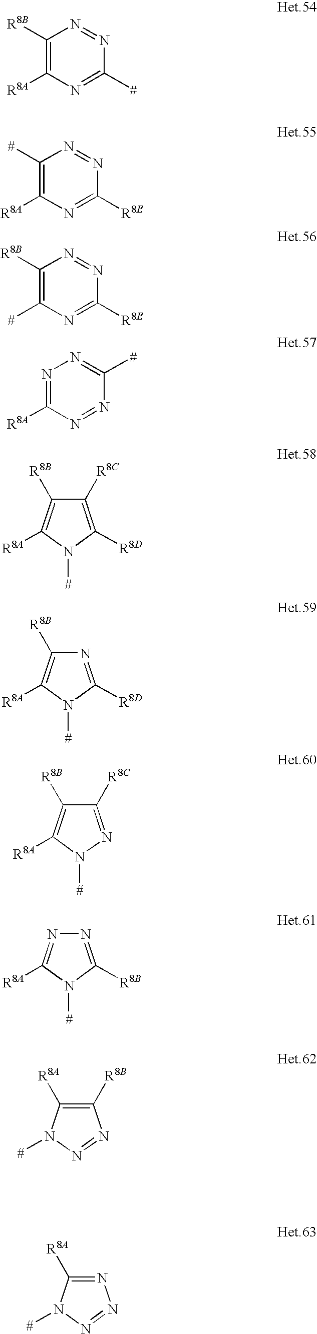

wherein # denotes the position of attachment in formula I, wherein the capital letter A, B, C, D and E joined to R⁸ denotes the position of R⁸ in formula I and wherein R^{8A}, R^{8B}, R^{8C}, R^{8D} and R^{8E}, independently of each other, are hydrogen or have one of the meanings given for R⁸ as given above.

Particular preferences are given to compounds of formula I, wherein, independently from one another, Het_{A} is selected from the radicals of formulae Het.1-Het.57 and/or Het_{B} is selected from the radicals of formulae Het.1-Het.63.

Preference is given to compounds of formula I, wherein Het_{A} and/or Het_{B} are selected independently from each other from the radicals of formulae Het.1, Het.2, Het.3, Het.4, Het.5, Het.6, Het.7, Het.8, Het.9, Het.10, Het.11, Het.12, Het.13, Het.14, Het.15, Het.16, Het.17, Het.18, Het.19, Het.20, Het.21, Het.22, Het.23, Het.24, Het.25, Het.26, Het.27, Het.28, Het.29, Het.30, Het.41, Het.42, Het.43, Het.49, Het.50 and Het.51.

Especially preference is given to compounds of formula I, wherein Het_{A} is selected from the radicals of formulae Het.1, Het.2, Het.3, Het.4, Het.22, Het.23, Het.24, Het.41, Het.42 and Het.43.

More preferably Het_{A} is selected from the radicals of formulae Het.1, Het.2, Het.3 and Het.4.

Preference is given to compounds of formula I, wherein Het_{B} is selected from the radicals of formulae Het.1, Het.2, Het.3, Het.4, Het.22, Het.23, Het.24, Het.41, Het.42 and Het.43.

More preferably Het_{B} is selected from the radicals of formulae Het.1, Het.2, Het.3 and Het.4.

Preferred are examples of radicals Het_{A} and/or Het_{B} in which R^{8A}, R^{8B}, R^{8C}, R^{8D} and R^{8E}, and R⁹, if present, have the meanings given in one row of table B.

TABLE H

| Radical | Het | R^{8A} | R^{8B} | R^{8C} | R^{8D} | R^{8E} | R⁹ |
|---|---|---|---|---|---|---|---|
| Het-R.1 | Het.1 | H | H | H | / | / | / |
| Het-R.2 | Het.1 | CH₃ | H | H | / | / | / |
| Het-R.3 | Het.1 | H | CH₃ | H | / | / | / |
| Het-R.4 | Het.1 | H | H | CH₃ | / | / | / |
| Het-R.5 | Het.1 | CF₃ | H | H | / | / | / |
| Het-R.6 | Het.1 | H | CF₃ | H | / | / | / |
| Het-R.7 | Het.1 | H | H | CF₃ | / | / | / |
| Het-R.8 | Het.1 | Cl | H | H | / | / | / |
| Het-R.9 | Het.1 | H | Cl | H | / | / | / |
| Het-R.10 | Het.1 | H | H | Cl | / | / | / |
| Het-R.11 | Het.1 | CH₃ | CH₃ | H | / | / | / |
| Het-R.12 | Het.1 | CH₃ | Cl | H | / | / | / |
| Het-R.13 | Het.1 | CH₃ | CF₃ | H | / | / | / |
| Het-R.14 | Het.1 | CH₃ | F | H | / | / | / |
| Het-R.15 | Het.1 | CF₃ | CH₃ | H | / | / | / |
| Het-R.16 | Het.1 | CF₃ | Cl | H | / | / | / |
| Het-R.17 | Het.1 | CF₃ | CF₃ | H | / | / | / |
| Het-R.18 | Het.1 | CF₃ | F | H | / | / | / |
| Het-R.19 | Het.1 | Cl | CH₃ | H | / | / | / |
| Het-R.20 | Het.1 | Cl | Cl | H | / | / | / |
| Het-R.21 | Het.1 | Cl | CF₃ | H | / | / | / |
| Het-R.22 | Het.1 | Cl | F | H | / | / | / |
| Het-R.23 | Het.1 | F | CH₃ | H | / | / | / |
| Het-R.24 | Het.1 | F | Cl | H | / | / | / |
| Het-R.25 | Het.1 | F | CF₃ | H | / | / | / |
| Het-R.26 | Het.1 | F | F | H | / | / | / |
| Het-R.27 | Het.1 | CH₃ | H | CH₃ | / | / | / |
| Het-R.28 | Het.1 | CH₃ | H | Cl | / | / | / |
| Het-R.29 | Het.1 | CH₃ | H | CF₃ | / | / | / |
| Het-R.30 | Het.1 | CH₃ | H | F | / | / | / |
| Het-R.31 | Het.1 | CF₃ | H | CH₃ | / | / | / |
| Het-R.32 | Het.1 | CF₃ | H | Cl | / | / | / |
| Het-R.33 | Het.1 | CF₃ | H | CF₃ | / | / | / |
| Het-R.34 | Het.1 | CF₃ | H | F | / | / | / |
| Het-R.35 | Het.1 | Cl | H | CH₃ | / | / | / |
| Het-R.36 | Het.1 | Cl | H | Cl | / | / | / |
| Het-R.37 | Het.1 | Cl | H | CF₃ | / | / | / |
| Het-R.38 | Het.1 | Cl | H | F | / | / | / |
| Het-R.39 | Het.1 | F | H | CH₃ | / | / | / |
| Het-R.40 | Het.1 | F | H | Cl | / | / | / |
| Het-R.41 | Het.1 | F | H | CF₃ | / | / | / |
| Het-R.42 | Het.1 | F | H | F | / | / | / |
| Het-R.43 | Het.1 | H | CH₃ | CH₃ | / | / | / |
| Het-R.44 | Het.1 | H | CH₃ | Cl | / | / | / |
| Het-R.45 | Het.1 | H | CH₃ | CF₃ | / | / | / |
| Het-R.46 | Het.1 | H | CH₃ | F | / | / | / |
| Het-R.47 | Het.1 | H | CF₃ | CH₃ | / | / | / |
| Het-R.48 | Het.1 | H | CF₃ | Cl | / | / | / |
| Het-R.49 | Het.1 | H | CF₃ | CF₃ | / | / | / |
| Het-R.50 | Het.1 | H | CF₃ | F | / | / | / |
| Het-R.51 | Het.1 | H | Cl | CH₃ | / | / | / |
| Het-R.52 | Het.1 | H | Cl | Cl | / | / | / |
| Het-R.53 | Het.1 | H | Cl | CF₃ | / | / | / |
| Het-R.54 | Het.1 | H | Cl | F | / | / | / |
| Het-R.55 | Het.1 | H | F | CH₃ | / | / | / |
| Het-R.56 | Het.1 | H | F | Cl | / | / | / |
| Het-R.57 | Het.1 | H | F | CF₃ | / | / | / |
| Het-R.58 | Het.1 | H | F | F | / | / | / |
| Het-R.59 | Het.2 | H | H | / | H | / | / |
| Het-R.60 | Het.2 | CH₃ | H | / | H | / | / |
| Het-R.61 | Het.2 | H | CH₃ | / | H | / | / |
| Het-R.62 | Het.2 | H | H | / | CH₃ | / | / |
| Het-R.63 | Het.2 | CF₃ | H | / | H | / | / |
| Het-R.64 | Het.2 | H | CF₃ | / | H | / | / |
| Het-R.65 | Het.2 | H | H | / | CF₃ | / | / |
| Het-R.66 | Het.2 | Cl | H | / | H | / | / |
| Het-R.67 | Het.2 | H | Cl | / | H | / | / |
| Het-R.68 | Het.2 | H | H | / | Cl | / | / |
| Het-R.69 | Het.2 | CH₃ | CH₃ | / | H | / | / |
| Het-R.70 | Het.2 | CH₃ | Cl | / | H | / | / |
| Het-R.71 | Het.2 | CH₃ | CF₃ | / | H | / | / |
| Het-R.72 | Het.2 | CH₃ | F | / | H | / | / |
| Het-R.73 | Het.2 | Cl | CH₃ | / | H | / | / |
| Het-R.74 | Het.2 | Cl | Cl | / | H | / | / |
| Het-R.75 | Het.2 | Cl | CF₃ | / | H | / | / |
| Het-R.76 | Het.2 | Cl | F | / | H | / | / |
| Het-R.77 | Het.2 | CF₃ | CH₃ | / | H | / | / |
| Het-R.78 | Het.2 | CF₃ | Cl | / | H | / | / |
| Het-R.79 | Het.2 | CF₃ | CF₃ | / | H | / | / |
| Het-R.80 | Het.2 | CF₃ | F | / | H | / | / |
| Het-R.81 | Het.2 | F | CH₃ | / | H | / | / |
| Het-R.82 | Het.2 | F | Cl | / | H | / | / |
| Het-R.83 | Het.2 | F | CF₃ | / | H | / | / |
| Het-R.84 | Het.2 | F | F | / | H | / | / |
| Het-R.85 | Het.2 | CH₃ | H | / | CH₃ | / | / |
| Het-R.86 | Het.2 | CH₃ | H | / | Cl | / | / |
| Het-R.87 | Het.2 | CH₃ | H | / | CF₃ | / | / |
| Het-R.88 | Het.2 | CH₃ | H | / | F | / | / |
| Het-R.89 | Het.2 | Cl | H | / | CH₃ | / | / |
| Het-R.90 | Het.2 | Cl | H | / | Cl | / | / |
| Het-R.91 | Het.2 | Cl | H | / | CF₃ | / | / |
| Het-R.92 | Het.2 | Cl | H | / | F | / | / |
| Het-R.93 | Het.2 | CF₃ | H | / | CH₃ | / | / |
| Het-R.94 | Het.2 | CF₃ | H | / | Cl | / | / |
| Het-R.95 | Het.2 | CF₃ | H | / | CF₃ | / | / |
| Het-R.96 | Het.2 | CF₃ | H | / | F | / | / |
| Het-R.97 | Het.2 | F | H | / | CH₃ | / | / |
| Het-R.98 | Het.2 | F | H | / | Cl | / | / |
| Het-R.99 | Het.2 | F | H | / | CF₃ | / | / |
| Het-R.100 | Het.2 | F | H | / | F | / | / |
| Het-R.101 | Het.2 | H | CH₃ | / | CH₃ | / | / |
| Het-R.102 | Het.2 | H | CH₃ | / | Cl | / | / |
| Het-R.103 | Het.2 | H | CH₃ | / | CF₃ | / | / |
| Het-R.104 | Het.2 | H | CH₃ | / | F | / | / |
| Het-R.105 | Het.2 | H | Cl | / | CH₃ | / | / |
| Het-R.106 | Het.2 | H | Cl | / | Cl | / | / |
| Het-R.107 | Het.2 | H | Cl | / | CF₃ | / | / |
| Het-R.108 | Het.2 | H | Cl | / | F | / | / |
| Het-R.109 | Het.2 | H | CF₃ | / | CH₃ | / | / |
| Het-R.110 | Het.2 | H | CF₃ | / | Cl | / | / |
| Het-R.111 | Het.2 | H | CF₃ | / | CF₃ | / | / |
| Het-R.112 | Het.2 | H | CF₃ | / | F | / | / |
| Het-R.113 | Het.2 | H | F | / | CH₃ | / | / |
| Het-R.114 | Het.2 | H | F | / | Cl | / | / |
| Het-R.115 | Het.2 | H | F | / | CF₃ | / | / |
| Het-R.116 | Het.2 | H | F | / | F | / | / |
| Het-R.117 | Het.3 | H | H | H | / | / | / |
| Het-R.118 | Het.3 | CH₃ | H | H | / | / | / |
| Het-R.119 | Het.3 | H | CH₃ | H | / | / | / |
| Het-R.120 | Het.3 | H | H | CH₃ | / | / | / |
| Het-R.121 | Het.3 | CF₃ | H | H | / | / | / |
| Het-R.122 | Het.3 | H | CF₃ | H | / | / | / |
| Het-R.123 | Het.3 | H | H | CF₃ | / | / | / |
| Het-R.124 | Het.3 | Cl | H | H | / | / | / |
| Het-R.125 | Het.3 | H | Cl | H | / | / | / |
| Het-R.126 | Het.3 | H | H | Cl | / | / | / |
| Het-R.127 | Het.3 | CH₃ | CH₃ | H | / | / | / |
| Het-R.128 | Het.3 | CH₃ | Cl | H | / | / | / |
| Het-R.129 | Het.3 | CH₃ | CF₃ | H | / | / | / |
| Het-R.130 | Het.3 | CH₃ | F | H | / | / | / |
| Het-R.131 | Het.3 | CF₃ | CH₃ | H | / | / | / |
| Het-R.132 | Het.3 | CF₃ | Cl | H | / | / | / |
| Het-R.133 | Het.3 | CF₃ | CF₃ | H | / | / | / |
| Het-R.134 | Het.3 | CF₃ | F | H | / | / | / |
| Het-R.135 | Het.3 | Cl | CH₃ | H | / | / | / |
| Het-R.136 | Het.3 | Cl | Cl | H | / | / | / |
| Het-R.137 | Het.3 | Cl | CF₃ | H | / | / | / |
| Het-R.138 | Het.3 | Cl | F | H | / | / | / |
| Het-R.139 | Het.3 | F | CH₃ | H | / | / | / |
| Het-R.140 | Het.3 | F | Cl | H | / | / | / |
| Het-R.141 | Het.3 | F | CF₃ | H | / | / | / |
| Het-R.142 | Het.3 | F | F | H | / | / | / |
| Het-R.143 | Het.3 | CH₃ | H | CH₃ | / | / | / |
| Het-R.144 | Het.3 | CH₃ | H | Cl | / | / | / |
| Het-R.145 | Het.3 | CH₃ | H | CF₃ | / | / | / |
| Het-R.146 | Het.3 | CH₃ | H | F | / | / | / |
| Het-R.147 | Het.3 | CF₃ | H | CH₃ | / | / | / |
| Het-R.148 | Het.3 | CF₃ | H | Cl | / | / | / |
| Het-R.149 | Het.3 | CF₃ | H | CF₃ | / | / | / |
| Het-R.150 | Het.3 | CF₃ | H | F | / | / | / |
| Het-R.151 | Het.3 | Cl | H | CH₃ | / | / | / |
| Het-R.152 | Het.3 | Cl | H | Cl | / | / | / |
| Het-R.153 | Het.3 | Cl | H | CF₃ | / | / | / |
| Het-R.154 | Het.3 | Cl | H | F | / | / | / |
| Het-R.155 | Het.3 | F | H | CH₃ | / | / | / |
| Het-R.156 | Het.3 | F | H | Cl | / | / | / |
| Het-R.157 | Het.3 | F | H | CF₃ | / | / | / |
| Het-R.158 | Het.3 | F | H | F | / | / | / |
| Het-R.159 | Het.3 | H | CH₃ | CH₃ | / | / | / |
| Het-R.160 | Het.3 | H | CH₃ | Cl | / | / | / |
| Het-R.161 | Het.3 | H | CH₃ | CF₃ | / | / | / |
| Het-R.162 | Het.3 | H | CH₃ | F | / | / | / |
| Het-R.163 | Het.3 | H | CF₃ | CH₃ | / | / | / |
| Het-R.164 | Het.3 | H | CF₃ | Cl | / | / | / |
| Het-R.165 | Het.3 | H | CF₃ | CF₃ | / | / | / |
| Het-R.166 | Het.3 | H | CF₃ | F | / | / | / |
| Het-R.167 | Het.3 | H | Cl | CH₃ | / | / | / |
| Het-R.168 | Het.3 | H | Cl | Cl | / | / | / |
| Het-R.169 | Het.3 | H | Cl | CF₃ | / | / | / |
| Het-R.170 | Het.3 | H | Cl | F | / | / | / |
| Het-R.171 | Het.3 | H | F | CH₃ | / | / | / |
| Het-R.172 | Het.3 | H | F | Cl | / | / | / |
| Het-R.173 | Het.3 | H | F | CF₃ | / | / | / |
| Het-R.174 | Het.3 | H | F | F | / | / | / |
| Het-R.175 | Het.4 | H | H | / | H | / | / |
| Het-R.176 | Het.4 | CH₃ | H | / | H | / | / |
| Het-R.177 | Het.4 | H | CH₃ | / | H | / | / |
| Het-R.178 | Het.4 | H | H | / | CH₃ | / | / |
| Het-R.179 | Het.4 | CF₃ | H | / | H | / | / |
| Het-R.180 | Het.4 | H | CF₃ | / | H | / | / |
| Het-R.181 | Het.4 | H | H | / | CF₃ | / | / |
| Het-R.182 | Het.4 | Cl | H | / | H | / | / |
| Het-R.183 | Het.4 | H | Cl | / | H | / | / |
| Het-R.184 | Het.4 | H | H | / | Cl | / | / |
| Het-R.185 | Het.4 | CH₃ | CH₃ | / | H | / | / |
| Het-R.186 | Het.4 | CH₃ | Cl | / | H | / | / |
| Het-R.187 | Het.4 | CH₃ | CF₃ | / | H | / | / |
| Het-R.188 | Het.4 | CH₃ | F | / | H | / | / |
| Het-R.189 | Het.4 | Cl | CH₃ | / | H | / | / |
| Het-R.190 | Het.4 | Cl | Cl | / | H | / | / |
| Het-R.191 | Het.4 | Cl | CF₃ | / | H | / | / |
| Het-R.192 | Het.4 | Cl | F | / | H | / | / |
| Het-R.193 | Het.4 | CF₃ | CH₃ | / | H | / | / |
| Het-R.194 | Het.4 | CF₃ | Cl | / | H | / | / |
| Het-R.195 | Het.4 | CF₃ | CF₃ | / | H | / | / |
| Het-R.196 | Het.4 | CF₃ | F | / | H | / | / |
| Het-R.197 | Het.4 | F | CH₃ | / | H | / | / |
| Het-R.198 | Het.4 | F | Cl | / | H | / | / |
| Het-R.199 | Het.4 | F | CF₃ | / | H | / | / |
| Het-R.200 | Het.4 | F | F | / | H | / | / |
| Het-R.201 | Het.4 | CH₃ | H | / | CH₃ | / | / |
| Het-R.202 | Het.4 | CH₃ | H | / | Cl | / | / |
| Het-R.203 | Het.4 | CH₃ | H | / | CF₃ | / | / |
| Het-R.204 | Het.4 | CH₃ | H | / | F | / | / |
| Het-R.205 | Het.4 | Cl | H | / | CH₃ | / | / |
| Het-R.206 | Het.4 | Cl | H | / | Cl | / | / |
| Het-R.207 | Het.4 | Cl | H | / | CF₃ | / | / |
| Het-R.208 | Het.4 | Cl | H | / | F | / | / |
| Het-R.209 | Het.4 | CF₃ | H | / | CH₃ | / | / |
| Het-R.210 | Het.4 | CF₃ | H | / | Cl | / | / |
| Het-R.211 | Het.4 | CF₃ | H | / | CF₃ | / | / |
| Het-R.212 | Het.4 | CF₃ | H | / | F | / | / |
| Het-R.213 | Het.4 | F | H | / | CH₃ | / | / |
| Het-R.214 | Het.4 | F | H | / | Cl | / | / |
| Het-R.215 | Het.4 | F | H | / | CF₃ | / | / |
| Het-R.216 | Het.4 | F | H | / | F | / | / |
| Het-R.217 | Het.4 | H | CH₃ | / | CH₃ | / | / |
| Het-R.218 | Het.4 | H | CH₃ | / | Cl | / | / |
| Het-R.219 | Het.4 | H | CH₃ | / | CF₃ | / | / |
| Het-R.220 | Het.4 | H | CH₃ | / | F | / | / |
| Het-R.221 | Het.4 | H | Cl | / | CH₃ | / | / |
| Het-R.222 | Het.4 | H | Cl | / | Cl | / | / |
| Het-R.223 | Het.4 | H | Cl | / | CF₃ | / | / |
| Het-R.224 | Het.4 | H | Cl | / | F | / | / |
| Het-R.225 | Het.4 | H | CF₃ | / | CH₃ | / | / |
| Het-R.226 | Het.4 | H | CF₃ | / | Cl | / | / |
| Het-R.227 | Het.4 | H | CF₃ | / | CF₃ | / | / |
| Het-R.228 | Het.4 | H | CF₃ | / | F | / | / |
| Het-R.229 | Het.4 | H | F | / | CH₃ | / | / |
| Het-R.230 | Het.4 | H | F | / | Cl | / | / |
| Het-R.231 | Het.4 | H | F | / | CF₃ | / | / |
| Het-R.232 | Het.4 | H | F | / | F | / | / |
| Het-R.233 | Het.5 | H | H | H | / | / | H |
| Het-R.234 | Het.5 | CH₃ | H | H | / | / | H |
| Het-R.235 | Het.5 | H | CH₃ | H | / | / | H |
| Het-R.236 | Het.5 | H | H | CH₃ | / | / | H |
| Het-R.237 | Het.5 | CF₃ | H | H | / | / | H |
| Het-R.238 | Het.5 | H | CF₃ | H | / | / | H |
| Het-R.239 | Het.5 | H | H | CF₃ | / | / | H |
| Het-R.240 | Het.5 | Cl | H | H | / | / | H |
| Het-R.241 | Het.5 | H | Cl | H | / | / | H |
| Het-R.242 | Het.5 | H | H | Cl | / | / | H |
| Het-R.243 | Het.5 | H | H | H | / | / | CH₃ |
| Het-R.244 | Het.5 | CH₃ | H | H | / | / | CH₃ |
| Het-R.245 | Het.5 | H | CH₃ | H | / | / | CH₃ |
| Het-R.246 | Het.5 | H | H | CH₃ | / | / | CH₃ |
| Het-R.247 | Het.5 | CF₃ | H | H | / | / | CH₃ |
| Het-R.248 | Het.5 | H | CF₃ | H | / | / | CH₃ |
| Het-R.249 | Het.5 | H | H | CF₃ | / | / | CH₃ |
| Het-R.250 | Het.5 | Cl | H | H | / | / | CH₃ |
| Het-R.251 | Het.5 | H | Cl | H | / | / | CH₃ |
| Het-R.252 | Het.5 | H | H | Cl | / | / | CH₃ |
| Het-R.253 | Het.5 | CH₃ | CH₃ | H | / | / | CH₃ |
| Het-R.254 | Het.5 | CH₃ | Cl | H | / | / | CH₃ |
| Het-R.255 | Het.5 | CH₃ | CF₃ | H | / | / | CH₃ |
| Het-R.256 | Het.5 | CH₃ | F | H | / | / | CH₃ |
| Het-R.257 | Het.5 | CF₃ | CH₃ | H | / | / | CH₃ |
| Het-R.258 | Het.5 | CF₃ | Cl | H | / | / | CH₃ |
| Het-R.259 | Het.5 | CF₃ | CF₃ | H | / | / | CH₃ |
| Het-R.260 | Het.5 | CF₃ | F | H | / | / | CH₃ |
| Het-R.261 | Het.5 | Cl | CH₃ | H | / | / | CH₃ |
| Het-R.262 | Het.5 | Cl | Cl | H | / | / | CH₃ |
| Het-R.263 | Het.5 | Cl | CF₃ | H | / | / | CH₃ |
| Het-R.264 | Het.5 | Cl | F | H | / | / | CH₃ |
| Het-R.265 | Het.5 | F | CH₃ | H | / | / | CH₃ |
| Het-R.266 | Het.5 | F | Cl | H | / | / | CH₃ |
| Het-R.267 | Het.5 | F | CF₃ | H | / | / | CH₃ |
| Het-R.268 | Het.5 | F | F | H | / | / | CH₃ |
| Het-R.269 | Het.5 | CH₃ | H | CH₃ | / | / | CH₃ |
| Het-R.270 | Het.5 | CH₃ | H | Cl | / | / | CH₃ |
| Het-R.271 | Het.5 | CH₃ | H | CF₃ | / | / | CH₃ |
| Het-R.272 | Het.5 | CH₃ | H | F | / | / | CH₃ |
| Het-R.273 | Het.5 | CF₃ | H | CH₃ | / | / | CH₃ |
| Het-R.274 | Het.5 | CF₃ | H | Cl | / | / | CH₃ |
| Het-R.275 | Het.5 | CF₃ | H | CF₃ | / | / | CH₃ |
| Het-R.276 | Het.5 | CF₃ | H | F | / | / | CH₃ |
| Het-R.277 | Het.5 | Cl | H | CH₃ | / | / | CH₃ |
| Het-R.278 | Het.5 | Cl | H | Cl | / | / | CH₃ |
| Het-R.279 | Het.5 | Cl | H | CF₃ | / | / | CH₃ |
| Het-R.280 | Het.5 | Cl | H | F | / | / | CH₃ |
| Het-R.281 | Het.5 | F | H | CH₃ | / | / | CH₃ |
| Het-R.282 | Het.5 | F | H | Cl | / | / | CH₃ |
| Het-R.283 | Het.5 | F | H | CF₃ | / | / | CH₃ |
| Het-R.284 | Het.5 | F | H | F | / | / | CH₃ |
| Het-R.285 | Het.5 | H | CH₃ | CH₃ | / | / | CH₃ |
| Het-R.286 | Het.5 | H | CH₃ | Cl | / | / | CH₃ |
| Het-R.287 | Het.5 | H | CH₃ | CF₃ | / | / | CH₃ |
| Het-R.288 | Het.5 | H | CH₃ | F | / | / | CH₃ |
| Het-R.289 | Het.5 | H | CF₃ | CH₃ | / | / | CH₃ |
| Het-R.290 | Het.5 | H | CF₃ | Cl | / | / | CH₃ |
| Het-R.291 | Het.5 | H | CF₃ | CF₃ | / | / | CH₃ |
| Het-R.292 | Het.5 | H | CF₃ | F | / | / | CH₃ |
| Het-R.293 | Het.5 | H | Cl | CH₃ | / | / | CH₃ |
| Het-R.294 | Het.5 | H | Cl | Cl | / | / | CH₃ |
| Het-R.295 | Het.5 | H | Cl | CF₃ | / | / | CH₃ |
| Het-R.296 | Het.5 | H | Cl | F | / | / | CH₃ |
| Het-R.297 | Het.5 | H | F | CH₃ | / | / | CH₃ |
| Het-R.298 | Het.5 | H | F | Cl | / | / | CH₃ |
| Het-R.299 | Het.5 | H | F | CF₃ | / | / | CH₃ |
| Het-R.300 | Het.5 | H | F | F | / | / | CH₃ |
| Het-R.301 | Het.6 | H | H | / | H | / | H |
| Het-R.302 | Het.6 | CH₃ | H | / | H | / | H |
| Het-R.303 | Het.6 | H | CH₃ | / | H | / | H |
| Het-R.304 | Het.6 | H | H | / | CH₃ | / | H |
| Het-R.305 | Het.6 | CF₃ | H | / | H | / | H |
| Het-R.306 | Het.6 | H | CF₃ | / | H | / | H |
| Het-R.307 | Het.6 | H | H | / | CF₃ | / | H |
| Het-R.308 | Het.6 | Cl | H | / | H | / | H |
| Het-R.309 | Het.6 | H | Cl | / | H | / | H |
| Het-R.310 | Het.6 | H | H | / | Cl | / | H |
| Het-R.311 | Het.6 | H | H | / | H | / | CH₃ |
| Het-R.312 | Het.6 | CH₃ | H | / | H | / | CH₃ |
| Het-R.313 | Het.6 | H | CH₃ | / | H | / | CH₃ |
| Het-R.314 | Het.6 | H | H | / | CH₃ | / | CH₃ |
| Het-R.315 | Het.6 | CF₃ | H | / | H | / | CH₃ |
| Het-R.316 | Het.6 | H | CF₃ | / | H | / | CH₃ |
| Het-R.317 | Het.6 | H | H | / | CF₃ | / | CH₃ |
| Het-R.318 | Het.6 | Cl | H | / | H | / | CH₃ |
| Het-R.319 | Het.6 | H | Cl | / | H | / | CH₃ |
| Het-R.320 | Het.6 | H | H | / | Cl | / | CH₃ |
| Het-R.321 | Het.6 | CH₃ | CH₃ | / | H | / | CH₃ |
| Het-R.322 | Het.6 | CH₃ | Cl | / | H | / | CH₃ |
| Het-R.323 | Het.6 | CH₃ | CF₃ | / | H | / | CH₃ |
| Het-R.324 | Het.6 | CH₃ | F | / | H | / | CH₃ |
| Het-R.325 | Het.6 | Cl | CH₃ | / | H | / | CH₃ |
| Het-R.326 | Het.6 | Cl | Cl | / | H | / | CH₃ |
| Het-R.327 | Het.6 | Cl | CF₃ | / | H | / | CH₃ |
| Het-R.328 | Het.6 | Cl | F | / | H | / | CH₃ |
| Het-R.329 | Het.6 | CF₃ | CH₃ | / | H | / | CH₃ |
| Het-R.330 | Het.6 | CF₃ | Cl | / | H | / | CH₃ |
| Het-R.331 | Het.6 | CF₃ | CF₃ | / | H | / | CH₃ |
| Het-R.332 | Het.6 | CF₃ | F | / | H | / | CH₃ |
| Het-R.333 | Het.6 | F | CH₃ | / | H | / | CH₃ |
| Het-R.334 | Het.6 | F | Cl | / | H | / | CH₃ |
| Het-R.335 | Het.6 | F | CF₃ | / | H | / | CH₃ |
| Het-R.336 | Het.6 | F | F | / | H | / | CH₃ |
| Het-R.337 | Het.6 | CH₃ | H | / | CH₃ | / | CH₃ |
| Het-R.338 | Het.6 | CH₃ | H | / | Cl | / | CH₃ |
| Het-R.339 | Het.6 | CH₃ | H | / | CF₃ | / | CH₃ |
| Het-R.340 | Het.6 | CH₃ | H | / | F | / | CH₃ |
| Het-R.341 | Het.6 | Cl | H | / | CH₃ | / | CH₃ |
| Het-R.342 | Het.6 | Cl | H | / | Cl | / | CH₃ |
| Het-R.343 | Het.6 | Cl | H | / | CF₃ | / | CH₃ |
| Het-R.344 | Het.6 | Cl | H | / | F | / | CH₃ |
| Het-R.345 | Het.6 | CF₃ | H | / | CH₃ | / | CH₃ |
| Het-R.346 | Het.6 | CF₃ | H | / | Cl | / | CH₃ |
| Het-R.347 | Het.6 | CF₃ | H | / | CF₃ | / | CH₃ |
| Het-R.348 | Het.6 | CF₃ | H | / | F | / | CH₃ |
| Het-R.349 | Het.6 | F | H | / | CH₃ | / | CH₃ |
| Het-R.350 | Het.6 | F | H | / | Cl | / | CH₃ |
| Het-R.351 | Het.6 | F | H | / | CF₃ | / | CH₃ |
| Het-R.352 | Het.6 | F | H | / | F | / | CH₃ |
| Het-R.353 | Het.6 | H | CH₃ | / | CH₃ | / | CH₃ |
| Het-R.354 | Het.6 | H | CH₃ | / | Cl | / | CH₃ |
| Het-R.355 | Het.6 | H | CH₃ | / | CF₃ | / | CH₃ |
| Het-R.356 | Het.6 | H | CH₃ | / | F | / | CH₃ |
| Het-R.357 | Het.6 | H | Cl | / | CH₃ | / | CH₃ |
| Het-R.358 | Het.6 | H | Cl | / | Cl | / | CH₃ |
| Het-R.359 | Het.6 | H | Cl | / | CF₃ | / | CH₃ |
| Het-R.360 | Het.6 | H | Cl | / | F | / | CH₃ |
| Het-R.361 | Het.6 | H | CF₃ | / | CH₃ | / | CH₃ |
| Het-R.362 | Het.6 | H | CF₃ | / | Cl | / | CH₃ |
| Het-R.363 | Het.6 | H | CF₃ | / | CF₃ | / | CH₃ |
| Het-R.364 | Het.6 | H | CF₃ | / | F | / | CH₃ |
| Het-R.365 | Het.6 | H | F | / | CH₃ | / | CH₃ |
| Het-R.366 | Het.6 | H | F | / | Cl | / | CH₃ |
| Het-R.367 | Het.6 | H | F | / | CF₃ | / | CH₃ |
| Het-R.368 | Het.6 | H | F | / | F | / | CH₃ |
| Het-R.369 | Het.7 | H | H | / | / | / | / |
| Het-R.370 | Het.7 | H | CH₃ | / | / | / | / |
| Het-R.371 | Het.7 | CH₃ | H | / | / | / | / |
| Het-R.372 | Het.7 | H | CF₃ | / | / | / | / |
| Het-R.373 | Het.7 | CF₃ | H | / | / | / | / |
| Het-R.374 | Het.7 | H | Cl | / | / | / | / |
| Het-R.375 | Het.7 | Cl | H | / | / | / | / |
| Het-R.376 | Het.8 | H | / | / | H | / | / |
| Het-R.377 | Het.8 | CH₃ | / | / | H | / | / |
| Het-R.378 | Het.8 | H | / | / | CH₃ | / | / |
| Het-R.379 | Het.8 | CF₃ | / | / | H | / | / |
| Het-R.380 | Het.8 | H | / | / | CF₃ | / | / |
| Het-R.381 | Het.8 | Cl | / | / | H | / | / |
| Het-R.382 | Het.8 | H | / | / | Cl | / | / |
| Het-R.383 | Het.9 | / | H | / | H | / | / |
| Het-R.384 | Het.9 | / | H | / | CH₃ | / | / |
| Het-R.385 | Het.9 | / | CH₃ | / | H | / | / |
| Het-R.386 | Het.9 | / | H | / | CF₃ | / | / |
| Het-R.387 | Het.9 | / | CF₃ | / | H | / | / |
| Het-R.388 | Het.9 | / | H | / | Cl | / | / |
| Het-R.389 | Het.9 | / | Cl | / | H | / | / |
| Het-R.390 | Het.10 | H | H | / | / | / | / |
| Het-R.391 | Het.10 | H | CH₃ | / | / | / | / |
| Het-R.392 | Het.10 | CH₃ | H | / | / | / | / |
| Het-R.393 | Het.10 | H | CF₃ | / | / | / | / |
| Het-R.394 | Het.10 | CF₃ | H | / | / | / | / |
| Het-R.395 | Het.10 | H | Cl | / | / | / | / |
| Het-R.396 | Het.10 | Cl | H | / | / | / | / |
| Het-R.397 | Het.11 | H | / | / | H | / | / |
| Het-R.398 | Het.11 | CH₃ | / | / | H | / | / |
| Het-R.399 | Het.11 | H | / | / | CH₃ | / | / |
| Het-R.400 | Het.11 | CF₃ | / | / | H | / | / |
| Het-R.401 | Het.11 | H | / | / | CF₃ | / | / |
| Het-R.402 | Het.11 | Cl | / | / | H | / | / |
| Het-R.403 | Het.11 | H | / | / | Cl | / | / |
| Het-R.404 | Het.12 | / | H | / | H | / | / |
| Het-R.405 | Het.12 | / | H | / | CH₃ | / | / |
| Het-R.406 | Het.12 | / | CH₃ | / | H | / | / |
| Het-R.407 | Het.12 | / | H | / | CF₃ | / | / |
| Het-R.408 | Het.12 | / | CF₃ | / | H | / | / |
| Het-R.409 | Het.12 | / | H | / | Cl | / | / |
| Het-R.410 | Het.12 | / | Cl | / | H | / | / |
| Het-R.411 | Het.13 | H | H | / | / | / | H |
| Het-R.412 | Het.13 | H | CH₃ | / | / | / | H |
| Het-R.413 | Het.13 | CH₃ | H | / | / | / | H |
| Het-R.414 | Het.13 | H | CF₃ | / | / | / | H |
| Het-R.415 | Het.13 | CF₃ | H | / | / | / | H |
| Het-R.416 | Het.13 | H | Cl | / | / | / | H |
| Het-R.417 | Het.13 | Cl | H | / | / | / | H |
| Het-R.418 | Het.13 | H | H | / | / | / | CH₃ |
| Het-R.419 | Het.13 | H | CH₃ | / | / | / | CH₃ |
| Het-R.420 | Het.13 | CH₃ | H | / | / | / | CH₃ |
| Het-R.421 | Het.13 | H | CF₃ | / | / | / | CH₃ |
| Het-R.422 | Het.13 | CF₃ | H | / | / | / | CH₃ |
| Het-R.423 | Het.13 | H | Cl | / | / | / | CH₃ |
| Het-R.424 | Het.13 | Cl | H | / | / | / | CH₃ |
| Het-R.425 | Het.14 | H | / | / | H | / | H |
| Het-R.426 | Het.14 | CH₃ | / | / | H | / | H |
| Het-R.427 | Het.14 | H | / | / | CH₃ | / | H |
| Het-R.428 | Het.14 | CF₃ | / | / | H | / | H |
| Het-R.429 | Het.14 | H | / | / | CF₃ | / | H |
| Het-R.430 | Het.14 | Cl | / | / | H | / | H |
| Het-R.431 | Het.14 | H | / | / | Cl | / | H |
| Het-R.432 | Het.14 | H | / | / | H | / | CH₃ |
| Het-R.433 | Het.14 | CH₃ | / | / | H | / | CH₃ |
| Het-R.434 | Het.14 | H | / | / | CH₃ | / | CH₃ |
| Het-R.435 | Het.14 | CF₃ | / | / | H | / | CH₃ |
| Het-R.436 | Het.14 | H | / | / | CF₃ | / | CH₃ |
| Het-R.437 | Het.14 | Cl | / | / | H | / | CH₃ |
| Het-R.438 | Het.14 | H | / | / | Cl | / | CH₃ |
| Het-R.439 | Het.15 | / | H | / | H | / | H |
| Het-R.440 | Het.15 | / | H | / | CH₃ | / | H |
| Het-R.441 | Het.15 | / | CH₃ | / | H | / | H |
| Het-R.442 | Het.15 | / | H | / | CF₃ | / | H |
| Het-R.443 | Het.15 | / | CF₃ | / | H | / | H |
| Het-R.444 | Het.15 | / | H | / | Cl | / | H |
| Het-R.445 | Het.15 | / | Cl | / | H | / | H |
| Het-R.446 | Het.15 | / | H | / | H | / | CH₃ |
| Het-R.447 | Het.15 | / | H | / | CH₃ | / | CH₃ |
| Het-R.448 | Het.15 | / | CH₃ | / | H | / | CH₃ |
| Het-R.449 | Het.15 | / | H | / | CF₃ | / | CH₃ |
| Het-R.450 | Het.15 | / | CF₃ | / | H | / | CH₃ |
| Het-R.451 | Het.15 | / | H | / | Cl | / | CH₃ |
| Het-R.452 | Het.15 | / | Cl | / | H | / | CH₃ |
| Het-R.453 | Het.16 | H | H | / | / | / | / |
| Het-R.454 | Het.16 | H | CH₃ | / | / | / | / |
| Het-R.455 | Het.16 | CH₃ | H | / | / | / | / |
| Het-R.456 | Het.16 | H | CF₃ | / | / | / | / |
| Het-R.457 | Het.16 | CF₃ | H | / | / | / | / |
| Het-R.458 | Het.16 | H | Cl | / | / | / | / |
| Het-R.459 | Het.16 | Cl | H | / | / | / | / |
| Het-R.460 | Het.17 | H | / | H | / | / | / |
| Het-R.461 | Het.17 | CH₃ | / | H | / | / | / |
| Het-R.462 | Het.17 | H | / | CH₃ | / | / | / |
| Het-R.463 | Het.17 | CF₃ | / | H | / | / | / |
| Het-R.464 | Het.17 | H | / | CF₃ | / | / | / |
| Het-R.465 | Het.17 | Cl | / | H | / | / | / |
| Het-R.466 | Het.17 | H | / | Cl | / | / | / |
| Het-R.467 | Het.18 | / | H | H | / | / | / |
| Het-R.468 | Het.18 | / | CH₃ | H | / | / | / |
| Het-R.469 | Het.18 | / | H | CH₃ | / | / | / |
| Het-R.470 | Het.18 | / | CF₃ | H | / | / | / |
| Het-R.471 | Het.18 | / | H | CF₃ | / | / | / |
| Het-R.472 | Het.18 | / | Cl | H | / | / | / |
| Het-R.473 | Het.18 | / | H | Cl | / | / | / |
| Het-R.474 | Het.19 | H | H | / | / | / | / |
| Het-R.475 | Het.19 | H | CH₃ | / | / | / | / |
| Het-R.476 | Het.19 | CH₃ | H | / | / | / | / |
| Het-R.477 | Het.19 | H | CF₃ | / | / | / | / |
| Het-R.478 | Het.19 | CF₃ | H | / | / | / | / |
| Het-R.479 | Het.19 | H | Cl | / | / | / | / |
| Het-R.480 | Het.19 | Cl | H | / | / | / | / |
| Het-R.481 | Het.20 | H | / | H | / | / | / |
| Het-R.482 | Het.20 | CH₃ | / | H | / | / | / |
| Het-R.483 | Het.20 | H | / | CH₃ | / | / | / |
| Het-R.484 | Het.20 | CF₃ | / | H | / | / | / |
| Het-R.485 | Het.20 | H | / | CF₃ | / | / | / |
| Het-R.486 | Het.20 | Cl | / | H | / | / | / |
| Het-R.487 | Het.20 | H | / | Cl | / | / | / |
| Het-R.488 | Het.21 | / | H | H | / | / | / |
| Het-R.489 | Het.21 | / | CH₃ | H | / | / | / |
| Het-R.490 | Het.21 | / | H | CH₃ | / | / | / |
| Het-R.491 | Het.21 | / | CF₃ | H | / | / | / |
| Het-R.492 | Het.21 | / | H | CF₃ | / | / | / |
| Het-R.493 | Het.21 | / | Cl | H | / | / | / |
| Het-R.494 | Het.21 | / | H | Cl | / | / | / |
| Het-R.495 | Het.22 | H | H | / | / | / | H |
| Het-R.496 | Het.22 | H | CH₃ | / | / | / | H |
| Het-R.497 | Het.22 | CH₃ | H | / | / | / | H |
| Het-R.498 | Het.22 | H | CF₃ | / | / | / | H |
| Het-R.499 | Het.22 | CF₃ | H | / | / | / | H |
| Het-R.500 | Het.22 | H | Cl | / | / | / | H |
| Het-R.501 | Het.22 | Cl | H | / | / | / | H |
| Het-R.502 | Het.22 | H | H | / | / | / | CH₃ |
| Het-R.503 | Het.22 | H | CH₃ | / | / | / | CH₃ |
| Het-R.504 | Het.22 | CH₃ | H | / | / | / | CH₃ |
| Het-R.505 | Het.22 | H | CF₃ | / | / | / | CH₃ |
| Het-R.506 | Het.22 | CF₃ | H | / | / | / | CH₃ |
| Het-R.507 | Het.22 | H | Cl | / | / | / | CH₃ |
| Het-R.508 | Het.22 | Cl | H | / | / | / | CH₃ |
| Het-R.509 | Het.23 | H | / | H | / | / | H |
| Het-R.510 | Het.23 | CH₃ | / | H | / | / | H |
| Het-R.511 | Het.23 | H | / | CH₃ | / | / | H |
| Het-R.512 | Het.23 | CF₃ | / | H | / | / | H |
| Het-R.513 | Het.23 | H | / | CF₃ | / | / | H |
| Het-R.514 | Het.23 | Cl | / | H | / | / | H |
| Het-R.515 | Het.23 | H | / | Cl | / | / | H |
| Het-R.516 | Het.23 | H | / | H | / | / | CH₃ |
| Het-R.517 | Het.23 | CH₃ | / | H | / | / | CH₃ |
| Het-R.518 | Het.23 | H | / | CH₃ | / | / | CH₃ |
| Het-R.519 | Het.23 | CF₃ | / | H | / | / | CH₃ |
| Het-R.520 | Het.23 | H | / | CF₃ | / | / | CH₃ |
| Het-R.521 | Het.23 | Cl | / | H | / | / | CH₃ |
| Het-R.522 | Het.23 | H | / | Cl | / | / | CH₃ |
| Het-R.523 | Het.24 | / | H | H | / | / | H |
| Het-R.524 | Het.24 | / | CH₃ | H | / | / | H |
| Het-R.525 | Het.24 | / | H | CH₃ | / | / | H |
| Het-R.526 | Het.24 | / | CF₃ | H | / | / | H |
| Het-R.527 | Het.24 | / | H | CF₃ | / | / | H |
| Het-R.528 | Het.24 | / | Cl | H | / | / | H |
| Het-R.529 | Het.24 | / | H | Cl | / | / | H |
| Het-R.530 | Het.24 | / | H | H | / | / | CH₃ |
| Het-R.531 | Het.24 | / | CH₃ | H | / | / | CH₃ |
| Het-R.532 | Het.24 | / | H | CH₃ | / | / | CH₃ |
| Het-R.533 | Het.24 | / | CF₃ | H | / | / | CH₃ |
| Het-R.534 | Het.24 | / | H | CF₃ | / | / | CH₃ |
| Het-R.535 | Het.24 | / | Cl | H | / | / | CH₃ |
| Het-R.536 | Het.24 | / | H | Cl | / | / | CH₃ |
| Het-R.537 | Het.25 | H | / | / | / | / | / |
| Het-R.538 | Het.25 | CH₃ | / | / | / | / | / |
| Het-R.539 | Het.25 | CF₃ | / | / | / | / | / |
| Het-R.540 | Het.25 | Cl | / | / | / | / | / |
| Het-R.541 | Het.26 | H | / | / | / | / | / |
| Het-R.542 | Het.26 | CH₃ | / | / | / | / | / |
| Het-R.543 | Het.26 | CF₃ | / | / | / | / | / |
| Het-R.544 | Het.26 | Cl | / | / | / | / | / |
| Het-R.545 | Het.27 | H | / | / | / | / | / |
| Het-R.546 | Het.27 | CH₃ | / | / | / | / | / |
| Het-R.547 | Het.27 | CF₃ | / | / | / | / | / |
| Het-R.548 | Het.27 | Cl | / | / | / | / | / |
| Het-R.549 | Het.28 | H | / | / | / | / | / |
| Het-R.550 | Het.28 | CH₃ | / | / | / | / | / |
| Het-R.551 | Het.28 | CF₃ | / | / | / | / | / |
| Het-R.552 | Het.28 | Cl | / | / | / | / | / |
| Het-R.553 | Het.29 | H | / | / | / | / | / |
| Het-R.554 | Het.29 | CH₃ | / | / | / | / | / |
| Het-R.555 | Het.29 | CF₃ | / | / | / | / | / |
| Het-R.556 | Het.29 | Cl | / | / | / | / | / |
| Het-R.557 | Het.30 | H | / | / | / | / | / |
| Het-R.558 | Het.30 | CH₃ | / | / | / | / | / |
| Het-R.559 | Het.30 | CF₃ | / | / | / | / | / |
| Het-R.560 | Het.30 | Cl | / | / | / | / | / |
| Het-R.561 | Het.31 | H | / | / | / | / | H |
| Het-R.562 | Het.31 | CH₃ | / | / | / | / | H |
| Het-R.563 | Het.31 | CF₃ | / | / | / | / | H |
| Het-R.564 | Het.31 | Cl | / | / | / | / | H |
| Het-R.565 | Het.31 | H | / | / | / | / | CH₃ |
| Het-R.566 | Het.31 | CH₃ | / | / | / | / | CH₃ |
| Het-R.567 | Het.31 | CF₃ | / | / | / | / | CH₃ |
| Het-R.568 | Het.31 | Cl | / | / | / | / | CH₃ |
| Het-R.569 | Het.32 | H | / | / | / | / | H |
| Het-R.570 | Het.32 | CH₃ | / | / | / | / | H |
| Het-R.571 | Het.32 | CF₃ | / | / | / | / | H |
| Het-R.572 | Het.32 | Cl | / | / | / | / | H |
| Het-R.573 | Het.32 | H | / | / | / | / | CH₃ |
| Het-R.574 | Het.32 | CH₃ | / | / | / | / | CH₃ |
| Het-R.575 | Het.32 | CF₃ | / | / | / | / | CH₃ |
| Het-R.576 | Het.32 | Cl | / | / | / | / | CH₃ |
| Het-R.577 | Het.33 | H | / | / | / | / | H |
| Het-R.578 | Het.33 | CH₃ | / | / | / | / | H |
| Het-R.579 | Het.33 | CF₃ | / | / | / | / | H |
| Het-R.580 | Het.33 | Cl | / | / | / | / | H |
| Het-R.581 | Het.33 | H | / | / | / | / | CH₃ |
| Het-R.582 | Het.33 | CH₃ | / | / | / | / | CH₃ |
| Het-R.583 | Het.33 | CF₃ | / | / | / | / | CH₃ |
| Het-R.584 | Het.33 | Cl | / | / | / | / | CH₃ |
| Het-R.585 | Het.34 | H | / | / | / | / | H |
| Het-R.586 | Het.34 | CH₃ | / | / | / | / | H |
| Het-R.587 | Het.34 | CF₃ | / | / | / | / | H |
| Het-R.588 | Het.34 | Cl | / | / | / | / | H |
| Het-R.589 | Het.34 | H | / | / | / | / | CH₃ |
| Het-R.590 | Het.34 | CH₃ | / | / | / | / | CH₃ |
| Het-R.591 | Het.34 | CF₃ | / | / | / | / | CH₃ |
| Het-R.592 | Het.34 | Cl | / | / | / | / | CH₃ |
| Het-R.593 | Het.35 | / | H | / | / | / | H |
| Het-R.594 | Het.35 | / | CH₃ | / | / | / | H |
| Het-R.595 | Het.35 | / | CF₃ | / | / | / | H |
| Het-R.596 | Het.35 | / | Cl | / | / | / | H |
| Het-R.597 | Het.35 | / | H | / | / | / | CH₃ |
| Het-R.598 | Het.35 | / | CH₃ | / | / | / | CH₃ |
| Het-R.599 | Het.35 | / | CF₃ | / | / | / | CH₃ |
| Het-R.600 | Het.35 | / | Cl | / | / | / | CH₃ |
| Het-R.601 | Het.36 | / | / | / | / | / | H |
| Het-R.602 | Het.36 | / | / | / | / | / | CH₃ |
| Het-R.603 | Het.37 | H | / | / | / | / | / |
| Het-R.604 | Het.37 | CH₃ | / | / | / | / | / |
| Het-R.605 | Het.37 | CF₃ | / | / | / | / | / |
| Het-R.606 | Het.37 | Cl | / | / | / | / | / |
| Het-R.607 | Het.38 | / | H | / | / | / | / |
| Het-R.608 | Het.38 | / | CH₃ | / | / | / | / |
| Het-R.609 | Het.38 | / | CF₃ | / | / | / | / |
| Het-R.610 | Het.38 | / | Cl | / | / | / | / |
| Het-R.611 | Het.39 | H | / | / | / | / | / |
| Het-R.612 | Het.39 | CH₃ | / | / | / | / | / |
| Het-R.613 | Het.39 | CF₃ | / | / | / | / | / |
| Het-R.614 | Het.39 | Cl | / | / | / | / | / |
| Het-R.615 | Het.40 | / | H | / | / | / | / |
| Het-R.616 | Het.40 | / | CH₃ | / | / | / | / |
| Het-R.617 | Het.40 | / | CF₃ | / | / | / | / |
| Het-R.618 | Het.40 | / | Cl | / | / | / | / |
| Het-R.619 | Het.41 | H | H | H | H | / | / |
| Het-R.620 | Het.41 | CH₃ | H | H | H | / | / |
| Het-R.621 | Het.41 | H | CH₃ | H | H | / | / |
| Het-R.622 | Het.41 | H | H | CH₃ | H | / | / |
| Het-R.623 | Het.41 | H | H | H | CH₃ | / | / |
| Het-R.624 | Het.41 | CF₃ | H | H | H | / | / |
| Het-R.625 | Het.41 | H | CF₃ | H | H | / | / |
| Het-R.626 | Het.41 | H | H | CF₃ | H | / | / |
| Het-R.627 | Het.41 | H | H | H | CF₃ | / | / |
| Het-R.628 | Het.41 | Cl | H | H | H | / | / |
| Het-R.629 | Het.41 | H | Cl | H | H | / | / |
| Het-R.630 | Het.41 | H | H | Cl | H | / | / |
| Het-R.631 | Het.41 | H | H | H | Cl | / | / |
| Het-R.632 | Het.41 | CH₃ | CH₃ | H | H | / | / |
| Het-R.633 | Het.41 | CH₃ | Cl | H | H | / | / |
| Het-R.634 | Het.41 | CH₃ | CF₃ | H | H | / | / |
| Het-R.635 | Het.41 | CH₃ | F | H | H | / | / |
| Het-R.636 | Het.41 | CF₃ | CH₃ | H | H | / | / |
| Het-R.637 | Het.41 | CF₃ | Cl | H | H | / | / |
| Het-R.638 | Het.41 | CF₃ | CF₃ | H | H | / | / |
| Het-R.639 | Het.41 | CF₃ | F | H | H | / | / |
| Het-R.640 | Het.41 | Cl | CH₃ | H | H | / | / |
| Het-R.641 | Het.41 | Cl | Cl | H | H | / | / |
| Het-R.642 | Het.41 | Cl | CF₃ | H | H | / | / |
| Het-R.643 | Het.41 | Cl | F | H | H | / | / |
| Het-R.644 | Het.41 | F | CH₃ | H | H | / | / |
| Het-R.645 | Het.41 | F | Cl | H | H | / | / |
| Het-R.646 | Het.41 | F | CF₃ | H | H | / | / |
| Het-R.647 | Het.41 | F | F | H | H | / | / |
| Het-R.648 | Het.41 | CH₃ | H | CH₃ | H | / | / |
| Het-R.649 | Het.41 | CH₃ | H | Cl | H | / | / |
| Het-R.650 | Het.41 | CH₃ | H | CF₃ | H | / | / |
| Het-R.651 | Het.41 | CH₃ | H | F | H | / | / |
| Het-R.652 | Het.41 | CF₃ | H | CH₃ | H | / | / |
| Het-R.653 | Het.41 | CF₃ | H | Cl | H | / | / |
| Het-R.654 | Het.41 | CF₃ | H | CF₃ | H | / | / |
| Het-R.655 | Het.41 | CF₃ | H | F | H | / | / |
| Het-R.656 | Het.41 | Cl | H | CH₃ | H | / | / |
| Het-R.657 | Het.41 | Cl | H | Cl | H | / | / |
| Het-R.658 | Het.41 | Cl | H | CF₃ | H | / | / |
| Het-R.659 | Het.41 | Cl | H | F | H | / | / |
| Het-R.660 | Het.41 | F | H | CH₃ | H | / | / |
| Het-R.661 | Het.41 | F | H | Cl | H | / | / |
| Het-R.662 | Het.41 | F | H | CF₃ | H | / | / |
| Het-R.663 | Het.41 | F | H | F | H | / | / |
| Het-R.664 | Het.41 | H | CH₃ | CH₃ | H | / | / |
| Het-R.665 | Het.41 | H | CH₃ | Cl | H | / | / |
| Het-R.666 | Het.41 | H | CH₃ | CF₃ | H | / | / |
| Het-R.667 | Het.41 | H | CH₃ | F | H | / | / |
| Het-R.668 | Het.41 | H | CF₃ | CH₃ | H | / | / |
| Het-R.669 | Het.41 | H | CF₃ | Cl | H | / | / |
| Het-R.670 | Het.41 | H | CF₃ | CF₃ | H | / | / |
| Het-R.671 | Het.41 | H | CF₃ | F | H | / | / |
| Het-R.672 | Het.41 | H | Cl | CH₃ | H | / | / |
| Het-R.673 | Het.41 | H | Cl | Cl | H | / | / |
| Het-R.674 | Het.41 | H | Cl | CF₃ | H | / | / |
| Het-R.675 | Het.41 | H | Cl | F | H | / | / |
| Het-R.676 | Het.41 | H | F | CH₃ | H | / | / |
| Het-R.677 | Het.41 | H | F | Cl | H | / | / |
| Het-R.678 | Het.41 | H | F | CF₃ | H | / | / |
| Het-R.679 | Het.41 | H | F | F | H | / | / |
| Het-R.680 | Het.41 | H | CH₃ | CH₃ | H | / | / |
| Het-R.681 | Het.41 | H | CH₃ | Cl | H | / | / |
| Het-R.682 | Het.41 | H | CH₃ | CF₃ | H | / | / |
| Het-R.683 | Het.41 | H | CH₃ | F | H | / | / |
| Het-R.684 | Het.41 | H | CF₃ | CH₃ | H | / | / |
| Het-R.685 | Het.41 | H | CF₃ | Cl | H | / | / |
| Het-R.686 | Het.41 | H | CF₃ | CF₃ | H | / | / |
| Het-R.687 | Het.41 | H | CF₃ | F | H | / | / |
| Het-R.688 | Het.41 | H | Cl | CH₃ | H | / | / |
| Het-R.689 | Het.41 | H | Cl | Cl | H | / | / |
| Het-R.690 | Het.41 | H | Cl | CF₃ | H | / | / |
| Het-R.691 | Het.41 | H | Cl | F | H | / | / |
| Het-R.692 | Het.41 | H | F | CH₃ | H | / | / |
| Het-R.693 | Het.41 | H | F | Cl | H | / | / |
| Het-R.694 | Het.41 | H | F | CF₃ | H | / | / |
| Het-R.695 | Het.41 | H | F | F | H | / | / |
| Het-R.696 | Het.41 | CH₃ | H | H | CH₃ | / | / |
| Het-R.697 | Het.41 | CH₃ | H | H | Cl | / | / |
| Het-R.698 | Het.41 | CH₃ | H | H | CF₃ | / | / |
| Het-R.699 | Het.41 | CH₃ | H | H | F | / | / |
| Het-R.700 | Het.41 | CF₃ | H | H | CH₃ | / | / |
| Het-R.701 | Het.41 | CF₃ | H | H | Cl | / | / |
| Het-R.702 | Het.41 | CF₃ | H | H | CF₃ | / | / |
| Het-R.703 | Het.41 | CF₃ | H | H | F | / | / |
| Het-R.704 | Het.41 | Cl | H | H | CH₃ | / | / |
| Het-R.705 | Het.41 | Cl | H | H | Cl | / | / |
| Het-R.706 | Het.41 | Cl | H | H | CF₃ | / | / |
| Het-R.707 | Het.41 | Cl | H | H | F | / | / |
| Het-R.708 | Het.41 | F | H | H | CH₃ | / | / |
| Het-R.709 | Het.41 | F | H | H | Cl | / | / |
| Het-R.710 | Het.41 | F | H | H | CF₃ | / | / |
| Het-R.711 | Het.41 | F | H | H | F | / | / |
| Het-R.712 | Het.41 | H | CH₃ | H | CH₃ | / | / |
| Het-R.713 | Het.41 | H | CH₃ | H | Cl | / | / |
| Het-R.714 | Het.41 | H | CH₃ | H | CF₃ | / | / |
| Het-R.715 | Het.41 | H | CH₃ | H | F | / | / |
| Het-R.716 | Het.41 | H | CF₃ | H | CH₃ | / | / |
| Het-R.717 | Het.41 | H | CF₃ | H | Cl | / | / |
| Het-R.718 | Het.41 | H | CF₃ | H | CF₃ | / | / |
| Het-R.719 | Het.41 | H | CF₃ | H | F | / | / |
| Het-R.720 | Het.41 | H | Cl | H | CH₃ | / | / |
| Het-R.721 | Het.41 | H | Cl | H | Cl | / | / |
| Het-R.722 | Het.41 | H | Cl | H | CF₃ | / | / |
| Het-R.723 | Het.41 | H | Cl | H | F | / | / |
| Het-R.724 | Het.41 | H | F | H | CH₃ | / | / |
| Het-R.725 | Het.41 | H | F | H | Cl | / | / |
| Het-R.726 | Het.41 | H | F | H | CF₃ | / | / |
| Het-R.727 | Het.41 | H | F | H | F | / | / |
| Het-R.728 | Het.42 | H | H | H | / | H | / |
| Het-R.729 | Het.42 | CH₃ | H | H | / | H | / |
| Het-R.730 | Het.42 | H | CH₃ | H | / | H | / |
| Het-R.731 | Het.42 | H | H | CH₃ | / | H | / |
| Het-R.732 | Het.42 | H | H | H | / | CH₃ | / |
| Het-R.733 | Het.42 | CF₃ | H | H | / | H | / |
| Het-R.734 | Het.42 | H | CF₃ | H | / | H | / |
| Het-R.735 | Het.42 | H | H | CF₃ | / | H | / |
| Het-R.736 | Het.42 | H | H | H | / | CF₃ | / |
| Het-R.737 | Het.42 | Cl | H | H | / | H | / |
| Het-R.738 | Het.42 | H | Cl | H | / | H | / |
| Het-R.739 | Het.42 | H | H | Cl | / | H | / |
| Het-R.740 | Het.42 | H | H | H | / | Cl | / |
| Het-R.741 | Het.42 | CH₃ | CH₃ | H | / | H | / |
| Het-R.742 | Het.42 | CH₃ | Cl | H | / | H | / |
| Het-R.743 | Het.42 | CH₃ | CF₃ | H | / | H | / |
| Het-R.744 | Het.42 | CH₃ | F | H | / | H | / |
| Het-R.745 | Het.42 | CF₃ | CH₃ | H | / | H | / |
| Het-R.746 | Het.42 | CF₃ | Cl | H | / | H | / |
| Het-R.747 | Het.42 | CF₃ | CF₃ | H | / | H | / |
| Het-R.748 | Het.42 | CF₃ | F | H | / | H | / |
| Het-R.749 | Het.42 | Cl | CH₃ | H | / | H | / |
| Het-R.750 | Het.42 | Cl | Cl | H | / | H | / |
| Het-R.751 | Het.42 | Cl | CF₃ | H | / | H | / |
| Het-R.752 | Het.42 | Cl | F | H | / | H | / |
| Het-R.753 | Het.42 | F | CH₃ | H | / | H | / |
| Het-R.754 | Het.42 | F | Cl | H | / | H | / |
| Het-R.755 | Het.42 | F | CF₃ | H | / | H | / |
| Het-R.756 | Het.42 | F | F | H | / | H | / |
| Het-R.757 | Het.42 | CH₃ | H | CH₃ | / | H | / |
| Het-R.758 | Het.42 | CH₃ | H | Cl | / | H | / |
| Het-R.759 | Het.42 | CH₃ | H | CF₃ | / | H | / |
| Het-R.760 | Het.42 | CH₃ | H | F | / | H | / |
| Het-R.761 | Het.42 | CF₃ | H | CH₃ | / | H | / |
| Het-R.762 | Het.42 | CF₃ | H | Cl | / | H | / |
| Het-R.763 | Het.42 | CF₃ | H | CF₃ | / | H | / |
| Het-R.764 | Het.42 | CF₃ | H | F | / | H | / |
| Het-R.765 | Het.42 | Cl | H | CH₃ | / | H | / |
| Het-R.766 | Het.42 | Cl | H | Cl | / | H | / |
| Het-R.767 | Het.42 | Cl | H | CF₃ | / | H | / |
| Het-R.768 | Het.42 | Cl | H | F | / | H | / |
| Het-R.769 | Het.42 | F | H | CH₃ | / | H | / |
| Het-R.770 | Het.42 | F | H | Cl | / | H | / |
| Het-R.771 | Het.42 | F | H | CF₃ | / | H | / |
| Het-R.772 | Het.42 | F | H | F | / | H | / |
| Het-R.773 | Het.42 | H | CH₃ | CH₃ | / | H | / |
| Het-R.774 | Het.42 | H | CH₃ | Cl | / | H | / |
| Het-R.775 | Het.42 | H | CH₃ | CF₃ | / | H | / |
| Het-R.776 | Het.42 | H | CH₃ | F | / | H | / |
| Het-R.777 | Het.42 | H | CF₃ | CH₃ | / | H | / |
| Het-R.778 | Het.42 | H | CF₃ | Cl | / | H | / |
| Het-R.779 | Het.42 | H | CF₃ | CF₃ | / | H | / |
| Het-R.780 | Het.42 | H | CF₃ | F | / | H | / |
| Het-R.781 | Het.42 | H | Cl | CH₃ | / | H | / |
| Het-R.782 | Het.42 | H | Cl | Cl | / | H | / |
| Het-R.783 | Het.42 | H | Cl | CF₃ | / | H | / |
| Het-R.784 | Het.42 | H | Cl | F | / | H | / |
| Het-R.785 | Het.42 | H | F | CH₃ | / | H | / |
| Het-R.786 | Het.42 | H | F | Cl | / | H | / |
| Het-R.787 | Het.42 | H | F | CF₃ | / | H | / |
| Het-R.788 | Het.42 | H | F | F | / | H | / |
| Het-R.789 | Het.42 | H | CH₃ | H | / | CH₃ | / |
| Het-R.790 | Het.42 | H | CH₃ | H | / | Cl | / |
| Het-R.791 | Het.42 | H | CH₃ | H | / | CF₃ | / |
| Het-R.792 | Het.42 | H | CH₃ | H | / | F | / |
| Het-R.793 | Het.42 | H | CF₃ | H | / | CH₃ | / |
| Het-R.794 | Het.42 | H | CF₃ | H | / | Cl | / |
| Het-R.795 | Het.42 | H | CF₃ | H | / | CF₃ | / |
| Het-R.796 | Het.42 | H | CF₃ | H | / | F | / |
| Het-R.797 | Het.42 | H | Cl | H | / | CH₃ | / |
| Het-R.798 | Het.42 | H | Cl | H | / | Cl | / |
| Het-R.799 | Het.42 | H | Cl | H | / | CF₃ | / |
| Het-R.800 | Het.42 | H | Cl | H | / | F | / |
| Het-R.801 | Het.42 | H | F | H | / | CH₃ | / |
| Het-R.802 | Het.42 | H | F | H | / | Cl | / |
| Het-R.803 | Het.42 | H | F | H | / | CF₃ | / |
| Het-R.804 | Het.42 | H | F | H | / | F | / |
| Het-R.805 | Het.43 | H | H | / | H | H | / |
| Het-R.806 | Het.43 | CH₃ | H | / | H | H | / |
| Het-R.807 | Het.43 | H | CH₃ | / | H | H | / |
| Het-R.808 | Het.43 | H | H | / | CH₃ | H | / |
| Het-R.809 | Het.43 | H | H | / | H | CH₃ | / |
| Het-R.810 | Het.43 | CF₃ | H | / | H | H | / |
| Het-R.811 | Het.43 | H | CF₃ | / | H | H | / |
| Het-R.812 | Het.43 | H | H | / | CF₃ | H | / |
| Het-R.813 | Het.43 | H | H | / | H | CF₃ | / |
| Het-R.814 | Het.43 | Cl | H | / | H | H | / |
| Het-R.815 | Het.43 | H | Cl | / | H | H | / |
| Het-R.816 | Het.43 | H | H | / | Cl | H | / |
| Het-R.817 | Het.43 | H | H | / | H | Cl | / |
| Het-R.818 | Het.43 | CH₃ | CH₃ | / | H | H | / |
| Het-R.819 | Het.43 | CH₃ | Cl | / | H | H | / |
| Het-R.820 | Het.43 | CH₃ | CF₃ | / | H | H | / |
| Het-R.821 | Het.43 | CH₃ | F | / | H | H | / |
| Het-R.822 | Het.43 | Cl | CH₃ | / | H | H | / |
| Het-R.823 | Het.43 | Cl | Cl | / | H | H | / |
| Het-R.824 | Het.43 | Cl | CF₃ | / | H | H | / |
| Het-R.825 | Het.43 | Cl | F | / | H | H | / |
| Het-R.826 | Het.43 | CF₃ | CH₃ | / | H | H | / |
| Het-R.827 | Het.43 | CF₃ | Cl | / | H | H | / |
| Het-R.828 | Het.43 | CF₃ | CF₃ | / | H | H | / |
| Het-R.829 | Het.43 | CF₃ | F | / | H | H | / |
| Het-R.830 | Het.43 | F | CH₃ | / | H | H | / |
| Het-R.831 | Het.43 | F | Cl | / | H | H | / |
| Het-R.832 | Het.43 | F | CF₃ | / | H | H | / |
| Het-R.833 | Het.43 | F | F | / | H | H | / |
| Het-R.834 | Het.43 | CH₃ | H | / | CH₃ | H | / |
| Het-R.835 | Het.43 | CH₃ | H | / | Cl | H | / |
| Het-R.836 | Het.43 | CH₃ | H | / | CF₃ | H | / |
| Het-R.837 | Het.43 | CH₃ | H | / | F | H | / |
| Het-R.838 | Het.43 | Cl | H | / | CH₃ | H | / |
| Het-R.839 | Het.43 | Cl | H | / | Cl | H | / |
| Het-R.840 | Het.43 | Cl | H | / | CF₃ | H | / |
| Het-R.841 | Het.43 | Cl | H | / | F | H | / |
| Het-R.842 | Het.43 | CF₃ | H | / | CH₃ | H | / |
| Het-R.843 | Het.43 | CF₃ | H | / | Cl | H | / |
| Het-R.844 | Het.43 | CF₃ | H | / | CF₃ | H | / |
| Het-R.845 | Het.43 | CF₃ | H | / | F | H | / |
| Het-R.846 | Het.43 | F | H | / | CH₃ | H | / |
| Het-R.847 | Het.43 | F | H | / | Cl | H | / |
| Het-R.848 | Het.43 | F | H | / | CF₃ | H | / |
| Het-R.849 | Het.43 | F | H | / | F | H | / |
| Het-R.850 | Het.43 | H | CH₃ | / | CH₃ | H | / |
| Het-R.851 | Het.43 | H | CH₃ | / | Cl | H | / |
| Het-R.852 | Het.43 | H | CH₃ | / | CF₃ | H | / |
| Het-R.853 | Het.43 | H | CH₃ | / | F | H | / |
| Het-R.854 | Het.43 | H | Cl | / | CH₃ | H | / |
| Het-R.855 | Het.43 | H | Cl | / | Cl | H | / |
| Het-R.856 | Het.43 | H | Cl | / | CF₃ | H | / |
| Het-R.857 | Het.43 | H | Cl | / | F | H | / |
| Het-R.858 | Het.43 | H | CF₃ | / | CH₃ | H | / |
| Het-R.859 | Het.43 | H | CF₃ | / | Cl | H | / |
| Het-R.860 | Het.43 | H | CF₃ | / | CF₃ | H | / |
| Het-R.861 | Het.43 | H | CF₃ | / | F | H | / |
| Het-R.862 | Het.43 | H | F | / | CH₃ | H | / |
| Het-R.863 | Het.43 | H | F | / | Cl | H | / |
| Het-R.864 | Het.43 | H | F | / | CF₃ | H | / |
| Het-R.865 | Het.43 | H | F | / | F | H | / |
| Het-R.866 | Het.43 | H | CH₃ | / | H | CH₃ | / |
| Het-R.867 | Het.43 | H | CH₃ | / | H | Cl | / |
| Het-R.868 | Het.43 | H | CH₃ | / | H | CF₃ | / |
| Het-R.869 | Het.43 | H | CH₃ | / | H | F | / |
| Het-R.870 | Het.43 | H | Cl | / | H | CH₃ | / |
| Het-R.871 | Het.43 | H | Cl | / | H | Cl | / |
| Het-R.872 | Het.43 | H | Cl | / | H | CF₃ | / |
| Het-R.873 | Het.43 | H | Cl | / | H | F | / |
| Het-R.874 | Het.43 | H | CF₃ | / | H | CH₃ | / |
| Het-R.875 | Het.43 | H | CF₃ | / | H | Cl | / |
| Het-R.876 | Het.43 | H | CF₃ | / | H | CF₃ | / |
| Het-R.877 | Het.43 | H | CF₃ | / | H | F | / |
| Het-R.878 | Het.43 | H | F | / | H | CH₃ | / |
| Het-R.879 | Het.43 | H | F | / | H | Cl | / |
| Het-R.880 | Het.43 | H | F | / | H | CF₃ | / |
| Het-R.881 | Het.43 | H | F | / | H | F | / |
| Het-R.882 | Het.43 | H | H | / | CH₃ | CH₃ | / |
| Het-R.883 | Het.43 | H | H | / | CH₃ | Cl | / |
| Het-R.884 | Het.43 | H | H | / | CH₃ | CF₃ | / |
| Het-R.885 | Het.43 | H | H | / | CH₃ | F | / |
| Het-R.886 | Het.43 | H | H | / | Cl | CH₃ | / |
| Het-R.887 | Het.43 | H | H | / | Cl | Cl | / |
| Het-R.888 | Het.43 | H | H | / | Cl | CF₃ | / |
| Het-R.889 | Het.43 | H | H | / | Cl | F | / |
| Het-R.890 | Het.43 | H | H | / | CF₃ | CH₃ | / |
| Het-R.891 | Het.43 | H | H | / | CF₃ | Cl | / |
| Het-R.892 | Het.43 | H | H | / | CF₃ | CF₃ | / |
| Het-R.893 | Het.43 | H | H | / | CF₃ | F | / |
| Het-R.894 | Het.43 | H | H | / | F | CH₃ | / |
| Het-R.895 | Het.43 | H | H | / | F | Cl | / |
| Het-R.896 | Het.43 | H | H | / | F | CF₃ | / |
| Het-R.897 | Het.43 | H | H | / | F | F | / |
| Het-R.898 | Het.44 | H | H | H | H | / | / |
| Het-R.899 | Het.44 | CH₃ | H | H | H | / | / |
| Het-R.900 | Het.44 | H | CH₃ | H | H | / | / |
| Het-R.901 | Het.44 | H | H | CH₃ | H | / | / |
| Het-R.902 | Het.44 | H | H | H | CH₃ | / | / |
| Het-R.903 | Het.44 | CF₃ | H | H | H | / | / |
| Het-R.904 | Het.44 | H | CF₃ | H | H | / | / |
| Het-R.905 | Het.44 | H | H | CF₃ | H | / | / |
| Het-R.906 | Het.44 | H | H | H | CF₃ | / | / |
| Het-R.907 | Het.44 | Cl | H | H | H | / | / |
| Het-R.908 | Het.44 | H | Cl | H | H | / | / |
| Het-R.909 | Het.44 | H | H | Cl | H | / | / |
| Het-R.910 | Het.44 | H | H | H | Cl | / | / |
| Het-R.911 | Het.45 | H | H | H | / | H | / |
| Het-R.912 | Het.45 | CH₃ | H | H | / | H | / |
| Het-R.913 | Het.45 | H | CH₃ | H | / | H | / |
| Het-R.914 | Het.45 | H | H | CH₃ | / | H | / |
| Het-R.915 | Het.45 | H | H | H | / | CH₃ | / |
| Het-R.916 | Het.45 | CF₃ | H | H | / | H | / |
| Het-R.917 | Het.45 | H | CF₃ | H | / | H | / |
| Het-R.918 | Het.45 | H | H | CF₃ | / | H | / |
| Het-R.919 | Het.45 | H | H | H | / | CF₃ | / |
| Het-R.920 | Het.45 | Cl | H | H | / | H | / |
| Het-R.921 | Het.45 | H | Cl | H | / | H | / |
| Het-R.922 | Het.45 | H | H | Cl | / | H | / |
| Het-R.923 | Het.45 | H | H | H | / | Cl | / |
| Het-R.924 | Het.46 | H | H | / | H | H | / |
| Het-R.925 | Het.46 | CH₃ | H | / | H | H | / |
| Het-R.926 | Het.46 | H | CH₃ | / | H | H | / |
| Het-R.927 | Het.46 | H | H | / | CH₃ | H | / |
| Het-R.928 | Het.46 | H | H | / | H | CH₃ | / |
| Het-R.929 | Het.46 | CF₃ | H | / | H | H | / |
| Het-R.930 | Het.46 | H | CF₃ | / | H | H | / |
| Het-R.931 | Het.46 | H | H | / | CF₃ | H | / |
| Het-R.932 | Het.46 | H | H | / | H | CF₃ | / |
| Het-R.933 | Het.46 | Cl | H | / | H | H | / |
| Het-R.934 | Het.46 | H | Cl | / | H | H | / |
| Het-R.935 | Het.46 | H | H | / | Cl | H | / |
| Het-R.936 | Het.46 | H | H | / | H | Cl | / |
| Het-R.937 | Het.47 | H | H | H | / | / | / |
| Het-R.938 | Het.47 | CH₃ | H | H | / | / | / |
| Het-R.939 | Het.47 | H | CH₃ | H | / | / | / |
| Het-R.940 | Het.47 | H | H | CH₃ | / | / | / |
| Het-R.941 | Het.47 | CF₃ | H | H | / | / | / |
| Het-R.942 | Het.47 | H | CF₃ | H | / | / | / |
| Het-R.943 | Het.47 | H | H | CF₃ | / | / | / |
| Het-R.944 | Het.47 | Cl | H | H | / | / | / |
| Het-R.945 | Het.47 | H | Cl | H | / | / | / |
| Het-R.946 | Het.47 | H | H | Cl | / | / | / |
| Het-R.947 | Het.48 | H | H | / | H | / | / |
| Het-R.948 | Het.48 | CH₃ | H | / | H | / | / |
| Het-R.949 | Het.48 | H | CH₃ | / | H | / | / |
| Het-R.950 | Het.48 | H | H | / | CH₃ | / | / |
| Het-R.951 | Het.48 | CF₃ | H | / | H | / | / |
| Het-R.952 | Het.48 | H | CF₃ | / | H | / | / |
| Het-R.953 | Het.48 | H | H | / | CF₃ | / | / |
| Het-R.954 | Het.48 | Cl | H | / | H | / | / |
| Het-R.955 | Het.48 | H | Cl | / | H | / | / |
| Het-R.956 | Het.48 | H | H | / | Cl | / | / |
| Het-R.957 | Het.49 | H | H | H | / | / | / |
| Het-R.958 | Het.49 | CH₃ | H | H | / | / | / |
| Het-R.959 | Het.49 | H | CH₃ | H | / | / | / |
| Het-R.960 | Het.49 | H | H | CH₃ | / | / | / |
| Het-R.961 | Het.49 | CF₃ | H | H | / | / | / |
| Het-R.962 | Het.49 | H | CF₃ | H | / | / | / |
| Het-R.963 | Het.49 | H | H | CF₃ | / | / | / |
| Het-R.964 | Het.49 | Cl | H | H | / | / | / |
| Het-R.965 | Het.49 | H | Cl | H | / | / | / |
| Het-R.966 | Het.49 | H | H | Cl | / | / | / |
| Het-R.967 | Het.50 | H | H | / | / | H | / |
| Het-R.968 | Het.50 | CH₃ | H | / | / | H | / |
| Het-R.969 | Het.50 | H | CH₃ | / | / | H | / |
| Het-R.970 | Het.50 | H | H | / | / | CH₃ | / |
| Het-R.971 | Het.50 | CF₃ | H | / | / | H | / |
| Het-R.972 | Het.50 | H | CF₃ | / | / | H | / |
| Het-R.973 | Het.50 | H | H | / | / | CF₃ | / |
| Het-R.974 | Het.50 | Cl | H | / | / | H | / |
| Het-R.975 | Het.50 | H | Cl | / | / | H | / |
| Het-R.976 | Het.50 | H | H | / | / | Cl | / |
| Het-R.977 | Het.51 | H | / | H | / | H | / |
| Het-R.978 | Het.51 | CH₃ | / | H | / | H | / |
| Het-R.979 | Het.51 | H | / | CH₃ | / | H | / |
| Het-R.980 | Het.51 | H | / | H | / | CH₃ | / |
| Het-R.981 | Het.51 | CF₃ | / | H | / | H | / |
| Het-R.982 | Het.51 | H | / | CF₃ | / | H | / |
| Het-R.983 | Het.51 | H | / | H | / | CF₃ | / |
| Het-R.984 | Het.51 | Cl | / | H | / | H | / |
| Het-R.985 | Het.51 | H | / | Cl | / | H | / |
| Het-R.986 | Het.51 | H | / | H | / | Cl | / |
| Het-R.987 | Het.52 | H | H | / | H | / | / |
| Het-R.988 | Het.52 | CH₃ | H | / | H | / | / |
| Het-R.989 | Het.52 | H | CH₃ | / | H | / | / |
| Het-R.990 | Het.52 | H | H | / | CH₃ | / | / |
| Het-R.991 | Het.52 | CF₃ | H | / | H | / | / |
| Het-R.992 | Het.52 | H | CF₃ | / | H | / | / |
| Het-R.993 | Het.52 | H | H | / | CF₃ | / | / |
| Het-R.994 | Het.52 | Cl | H | / | H | / | / |
| Het-R.995 | Het.52 | H | Cl | / | H | / | / |
| Het-R.996 | Het.52 | H | H | / | Cl | / | / |
| Het-R.997 | Het.53 | H | / | H | / | / | / |
| Het-R.998 | Het.53 | CH₃ | / | H | / | / | / |
| Het-R.999 | Het.53 | H | / | CH₃ | / | / | / |
| Het-R.1000 | Het.53 | CF₃ | / | H | / | / | / |
| Het-R.1001 | Het.53 | H | / | CF₃ | / | / | / |
| Het-R.1002 | Het.53 | Cl | / | H | / | / | / |
| Het-R.1003 | Het.53 | H | / | Cl | / | / | / |
| Het-R.1004 | Het.54 | H | H | / | / | / | / |
| Het-R.1005 | Het.54 | H | CH₃ | / | / | / | / |
| Het-R.1006 | Het.54 | CH₃ | H | / | / | / | / |
| Het-R.1007 | Het.54 | H | CF₃ | / | / | / | / |
| Het-R.1008 | Het.54 | CF₃ | H | / | / | / | / |
| Het-R.1009 | Het.54 | H | Cl | / | / | / | / |
| Het-R.1010 | Het.54 | Cl | H | / | / | / | / |
| Het-R.1011 | Het.55 | H | / | / | / | H | / |
| Het-R.1012 | Het.55 | CH₃ | / | / | / | H | / |
| Het-R.1013 | Het.55 | H | / | / | / | CH₃ | / |
| Het-R.1014 | Het.55 | CF₃ | / | / | / | H | / |
| Het-R.1015 | Het.55 | H | / | / | / | CF₃ | / |
| Het-R.1016 | Het.55 | Cl | / | / | / | H | / |
| Het-R.1017 | Het.55 | H | / | / | / | Cl | / |
| Het-R.1018 | Het.56 | / | H | / | / | H | / |
| Het-R.1019 | Het.56 | / | CH₃ | / | / | H | / |
| Het-R.1020 | Het.56 | / | H | / | / | CH₃ | / |
| Het-R.1021 | Het.56 | / | CF₃ | / | / | H | / |
| Het-R.1022 | Het.56 | / | H | / | / | CF₃ | / |
| Het-R.1023 | Het.56 | / | Cl | / | / | H | / |
| Het-R.1024 | Het.56 | / | H | / | / | Cl | / |
| Het-R.1025 | Het.57 | H | / | / | / | / | / |
| Het-R.1026 | Het.57 | CH₃ | / | / | / | / | / |
| Het-R.1027 | Het.57 | CF₃ | / | / | / | / | / |
| Het-R.1028 | Het.57 | Cl | / | / | / | / | / |

Apart from that,

R^{a} and R^{b} are, independently of each other, preferably selected from hydrogen and C₁-C₆-alkyl.
R^{c} is preferably C₁-C₆-alkyl.
Y is preferably a single bond, O, S or methylen.
Ar is preferably phenyl, a 5- or 6-membered monocyclic heteroaromatic ring.
Cy is preferably cyclohexyl.

Particular preference is given to those compounds I, wherein

R¹, R², R³ are hydrogen;
A is A² wherein R^{4a}, R^{4b}, R^{4c}, R^{d} are hydrogen and X is sulfur; and
R⁶ is hydrogen.

Particular preference is also given to those compounds I, wherein

R¹, R², R³ are hydrogen;
A is A² wherein R^{4a}, R^{4b}, R^{4c}, R^{d} are hydrogen and X is O; and
R⁶ is hydrogen.

Particular preference is also given to those compounds I, wherein

R¹, R², R³ are hydrogen;
A is A² wherein R^{4a}, R^{4b}, R^{4c}, R^{d} are hydrogen and X is NH; and
R⁶ is hydrogen.

Particular preference is also given to those compounds I, wherein

R¹, R², R³ are hydrogen;
A is A² wherein R^{4a}, R^{4b}, R^{4c}, R^{d} are hydrogen and X is N—CH₃; and
R⁶ is hydrogen.

Particular preference is also given to those compounds I, wherein

R¹, R², R³ are hydrogen;
A is A² wherein R^{4a}, R^{4b}, R^{4c}, R^{d} are hydrogen and X is N—C(O)CH₃; and
R⁶ is hydrogen.

Examples of preferred compounds I, wherein R¹, R², R³ are hydrogen, A is a radical A² with R^{4a}, R^{4b}, R^{4c} and R^{4d} being hydrogen, X═S and R⁶ is hydrogen, are described in the following tables 1 to 232 (hereinafter also referred to as compounds Ip):

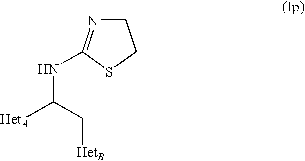

Table 1. Compounds of the formula Ip, wherein Het_{A} corresponds to Het-R.1 as defined in table H, and Het_{B} corresponds for each individual compound to one row of table H.
Table 2. Compounds of the formula Ip, wherein Het_{A} corresponds to Het-R.2 as defined in table H, and Het_{B} corresponds for each individual compound to one row of table H.
Table 3. Compounds of the formula Ip, wherein Het_{A} corresponds to Het-R.3 as defined in table H, and Het_{B} corresponds for each individual compound to one row of table H.
Table 4. Compounds of the formula Ip, wherein Het_{A} corresponds to Het-R.4 as defined in table H, and Het_{B} corresponds for each individual compound to one row of table H.
Table 5. Compounds of the formula Ip, wherein Het_{A} corresponds to Het-R.5 as defined in table H, and Het_{B} corresponds for each individual compound to one row of table H.
Table 6. Compounds of the formula Ip, wherein Het_{A} corresponds to Het-R.6 as defined in table H, and Het_{B} corresponds for each individual compound to one row of table H.
Table 7. Compounds of the formula Ip, wherein Het_{A} corresponds to Het-R.7 as defined in table H, and Het_{B} corresponds for each individual compound to one row of table H.
Table 8. Compounds of the formula Ip, wherein Het_{A} corresponds to Het-R.8 as defined in table H, and Het_{B} corresponds for each individual compound to one row of table H.
Table 9. Compounds of the formula Ip, wherein Het_{A} corresponds to Het-R.9 as defined in table H, and Het_{B} corresponds for each individual compound to one row of table H.
Table 10. Compounds of the formula Ip, wherein Het_{A} corresponds to Het-R.10 as defined in table H, and Het_{B} corresponds for each individual compound to one row of table H.
Table 11. Compounds of the formula Ip, wherein Het_{A} corresponds to Het-R.11 as defined in table H, and Het_{B} corresponds for each individual compound to one row of table H.
Table 12. Compounds of the formula Ip, wherein Het_{A} corresponds to Het-R.12 as defined in table H, and Het_{B} corresponds for each individual compound to one row of table H.
Table 13. Compounds of the formula Ip, wherein Het_{A} corresponds to Het-R.13 as defined in table H, and Het_{B} corresponds for each individual compound to one row of table H.
Table 14. Compounds of the formula Ip, wherein Het_{A} corresponds to Het-R.14 as defined in table H, and Het_{B} corresponds for each individual compound to one row of table H.
Table 15. Compounds of the formula Ip, wherein Het_{A} corresponds to Het-R.15 as defined in table H, and Het_{B} corresponds for each individual compound to one row of table H.
Table 16. Compounds of the formula Ip, wherein Het_{A} corresponds to Het-R.16 as defined in table H, and Het_{B} corresponds for each individual compound to one row of table H.
Table 17. Compounds of the formula Ip, wherein Het_{A} corresponds to Het-R.17 as defined in table H, and Het_{B} corresponds for each individual compound to one row of table H.
Table 18. Compounds of the formula Ip, wherein Het_{A} corresponds to Het-R.18 as defined in table H, and Het_{B} corresponds for each individual compound to one row of table H.
Table 19. Compounds of the formula Ip, wherein Het_{A} corresponds to Het-R.19 as defined in table H, and Het_{B} corresponds for each individual compound to one row of table H.
Table 20. Compounds of the formula Ip, wherein Het_{A} corresponds to Het-R.20 as defined in table H, and Het_{B} corresponds for each individual compound to one row of table H.
Table 21. Compounds of the formula Ip, wherein Het_{A} corresponds to Het-R.21 as defined in table H, and Het_{B} corresponds for each individual compound to one row of table H.
Table 22. Compounds of the formula Ip, wherein Het_{A} corresponds to Het-R.22 as defined in table H, and Het_{B} corresponds for each individual compound to one row of table H.
Table 23. Compounds of the formula Ip, wherein Het_{A} corresponds to Het-R.23 as defined in table H, and Het_{B} corresponds for each individual compound to one row of table H.
Table 24. Compounds of the formula Ip, wherein Het_{A} corresponds to Het-R.24 as defined in table H, and Het_{B} corresponds for each individual compound to one row of table H.
Table 25. Compounds of the formula Ip, wherein Het_{A} corresponds to Het-R.25 as defined in table H, and Het_{B} corresponds for each individual compound to one row of table H.
Table 26. Compounds of the formula Ip, wherein Het_{A} corresponds to Het-R.26 as defined in table H, and Het_{B} corresponds for each individual compound to one row of table H.
Table 27. Compounds of the formula Ip, wherein Het_{A} corresponds to Het-R.27 as defined in table H, and Het_{B} corresponds for each individual compound to one row of table H.
Table 28. Compounds of the formula Ip, wherein Het_{A} corresponds to Het-R.28 as defined in table H, and Het_{B} corresponds for each individual compound to one row of table H.
Table 29. Compounds of the formula Ip, wherein Het_{A} corresponds to Het-R.29 as defined in table H, and Het_{B} corresponds for each individual compound to one row of table H.
Table 30. Compounds of the formula Ip, wherein Het_{A} corresponds to Het-R.30 as defined in table H, and Het_{B} corresponds for each individual compound to one row of table H.
Table 31. Compounds of the formula Ip, wherein Het_{A} corresponds to Het-R.31 as defined in table H, and Het_{B} corresponds for each individual compound to one row of table H.
Table 32. Compounds of the formula Ip, wherein Het_{A} corresponds to Het-R.32 as defined in table H, and Het_{B} corresponds for each individual compound to one row of table H.
Table 33. Compounds of the formula Ip, wherein Het_{A} corresponds to Het-R.33 as defined in table H, and Het_{B} corresponds for each individual compound to one row of table H.
Table 34. Compounds of the formula Ip, wherein Het_{A} corresponds to Het-R.34 as defined in table H, and Het_{B} corresponds for each individual compound to one row of table H.
Table 35. Compounds of the formula Ip, wherein Het_{A} corresponds to Het-R.35 as defined in table H, and Het_{B} corresponds for each individual compound to one row of table H.
Table 36. Compounds of the formula Ip, wherein Het_{A} corresponds to Het-R.36 as defined in table H, and Het_{B} corresponds for each individual compound to one row of table H.
Table 37. Compounds of the formula Ip, wherein Het_{A} corresponds to Het-R.37 as defined in table H, and Het_{B} corresponds for each individual compound to one row of table H.
Table 38. Compounds of the formula Ip, wherein Het_{A} corresponds to Het-R.38 as defined in table H, and Het_{B} corresponds for each individual compound to one row of table H.
Table 39. Compounds of the formula Ip, wherein Het_{A} corresponds to Het-R.39 as defined in table H, and Het_{B} corresponds for each individual compound to one row of table H.
Table 40. Compounds of the formula Ip, wherein Het_{A} corresponds to Het-R.40 as defined in table H, and Het_{B} corresponds for each individual compound to one row of table H.
Table 41. Compounds of the formula Ip, wherein Het_{A} corresponds to Het-R.41 as defined in table H, and Het_{B} corresponds for each individual compound to one row of table H.
Table 42. Compounds of the formula Ip, wherein Het_{A} corresponds to Het-R.42 as defined in table H, and Het_{B} corresponds for each individual compound to one row of table H.
Table 43. Compounds of the formula Ip, wherein Het_{A} corresponds to Het-R.43 as defined in table H, and Het_{B} corresponds for each individual compound to one row of table H.
Table 44. Compounds of the formula Ip, wherein Het_{A} corresponds to Het-R.44 as defined in table H, and Het_{B} corresponds for each individual compound to one row of table H.
Table 45. Compounds of the formula Ip, wherein Het_{A} corresponds to Het-R.45 as defined in table H, and Het_{B} corresponds for each individual compound to one row of table H.
Table 46. Compounds of the formula Ip, wherein Het_{A} corresponds to Het-R.46 as defined in table H, and Het_{B} corresponds for each individual compound to one row of table H.
Table 47. Compounds of the formula Ip, wherein Het_{A} corresponds to Het-R.47 as defined in table H, and Het_{B} corresponds for each individual compound to one row of table H.
Table 48. Compounds of the formula Ip, wherein Het_{A} corresponds to Het-R.48 as defined in table H, and Het_{B} corresponds for each individual compound to one row of table H.
Table 49. Compounds of the formula Ip, wherein Het_{A} corresponds to Het-R.49 as defined in table H, and Het_{B} corresponds for each individual compound to one row of table H.
Table 50. Compounds of the formula Ip, wherein Het_{A} corresponds to Het-R.50 as defined in table H, and Het_{B} corresponds for each individual compound to one row of table H.
Table 51. Compounds of the formula Ip, wherein Het_{A} corresponds to Het-R.51 as defined in table H, and Het_{B} corresponds for each individual compound to one row of table H.
Table 52. Compounds of the formula Ip, wherein Het_{A} corresponds to Het-R.52 as defined in table H, and Het_{B} corresponds for each individual compound to one row of table H.
Table 53. Compounds of the formula Ip, wherein Het_{A} corresponds to Het-R.53 as defined in table H, and Het_{B} corresponds for each individual compound to one row of table H.
Table 54. Compounds of the formula Ip, wherein Het_{A} corresponds to Het-R.54 as defined in table H, and Het_{B} corresponds for each individual compound to one row of table H.
Table 55. Compounds of the formula Ip, wherein Het_{A} corresponds to Het-R.55 as defined in table H, and Het_{B} corresponds for each individual compound to one row of table H.
Table 56. Compounds of the formula Ip, wherein Het_{A} corresponds to Het-R.56 as defined in table H, and Het_{B} corresponds for each individual compound to one row of table H.
Table 57. Compounds of the formula Ip, wherein Het_{A} corresponds to Het-R.57 as defined in table H, and Het_{B} corresponds for each individual compound to one row of table H.
Table 58. Compounds of the formula Ip, wherein Het_{A} corresponds to Het-R.58 as defined in table H, and Het_{B} corresponds for each individual compound to one row of table H.
Table 59. Compounds of the formula Ip, wherein Het_{A} corresponds to Het-R.59 as defined in table H, and Het_{B} corresponds for each individual compound to one row of table H.
Table 60. Compounds of the formula Ip, wherein Het_{A} corresponds to Het-R.60 as defined in table H, and Het_{B} corresponds for each individual compound to one row of table H.
Table 61. Compounds of the formula Ip, wherein Het_{A} corresponds to Het-R.61 as defined in table H, and Het_{B} corresponds for each individual compound to one row of table H.
Table 62. Compounds of the formula Ip, wherein Het_{A} corresponds to Het-R.62 as defined in table H, and Het_{B} corresponds for each individual compound to one row of table H.
Table 63. Compounds of the formula Ip, wherein Het_{A} corresponds to Het-R.63 as defined in table H, and Het_{B} corresponds for each individual compound to one row of table H.
Table 64. Compounds of the formula Ip, wherein Het_{A} corresponds to Het-R.64 as defined in table H, and Het_{B} corresponds for each individual compound to one row of table H.
Table 65. Compounds of the formula Ip, wherein Het_{A} corresponds to Het-R.65 as defined in table H, and Het_{B} corresponds for each individual compound to one row of table H.
Table 66. Compounds of the formula Ip, wherein Het_{A} corresponds to Het-R.66 as defined in table H, and Het_{B} corresponds for each individual compound to one row of table H.
Table 67. Compounds of the formula Ip, wherein Het_{A} corresponds to Het-R.67 as defined in table H, and Het_{B} corresponds for each individual compound to one row of table H.
Table 68. Compounds of the formula Ip, wherein Het_{A} corresponds to Het-R.68 as defined in table H, and Het_{B} corresponds for each individual compound to one row of table H.
Table 69. Compounds of the formula Ip, wherein Het_{A} corresponds to Het-R.69 as defined in table H, and Het_{B} corresponds for each individual compound to one row of table H.
Table 70. Compounds of the formula Ip, wherein Het_{A} corresponds to Het-R.70 as defined in table H, and Het_{B} corresponds for each individual compound to one row of table H.
Table 71. Compounds of the formula Ip, wherein Het_{A} corresponds to Het-R.71 as defined in table H, and Het_{B} corresponds for each individual compound to one row of table H.
Table 72. Compounds of the formula Ip, wherein Het_{A} corresponds to Het-R.72 as defined in table H, and Het_{B} corresponds for each individual compound to one row of table H.
Table 73. Compounds of the formula Ip, wherein Het_{A} corresponds to Het-R.73 as defined in table H, and Het_{B} corresponds for each individual compound to one row of table H.
Table 74. Compounds of the formula Ip, wherein Het_{A} corresponds to Het-R.74 as defined in table H, and Het_{B} corresponds for each individual compound to one row of table H.
Table 75. Compounds of the formula Ip, wherein Het_{A} corresponds to Het-R.75 as defined in table H, and Het_{B} corresponds for each individual compound to one row of table H.
Table 76. Compounds of the formula Ip, wherein Het_{A} corresponds to Het-R.76 as defined in table H, and Het_{B} corresponds for each individual compound to one row of table H.
Table 77. Compounds of the formula Ip, wherein Het_{A} corresponds to Het-R.77 as defined in table H, and Het_{B} corresponds for each individual compound to one row of table H.
Table 78. Compounds of the formula Ip, wherein Het_{A} corresponds to Het-R.78 as defined in table H, and Het_{B} corresponds for each individual compound to one row of table H.
Table 79. Compounds of the formula Ip, wherein Het_{A} corresponds to Het-R.79 as defined in table H, and Het_{B} corresponds for each individual compound to one row of table H.
Table 80. Compounds of the formula Ip, wherein Het_{A} corresponds to Het-R.80 as defined in table H, and Het_{B} corresponds for each individual compound to one row of table H.
Table 81. Compounds of the formula Ip, wherein Het_{A} corresponds to Het-R.81 as defined in table H, and Het_{B} corresponds for each individual compound to one row of table H.
Table 82. Compounds of the formula Ip, wherein Het_{A} corresponds to Het-R.82 as defined in table H, and Het_{B} corresponds for each individual compound to one row of table H.
Table 83. Compounds of the formula Ip, wherein Het_{A} corresponds to Het-R.83 as defined in table H, and Het_{B} corresponds for each individual compound to one row of table H.
Table 84. Compounds of the formula Ip, wherein Het_{A} corresponds to Het-R.84 as defined in table H, and Het_{B} corresponds for each individual compound to one row of table H.
Table 85. Compounds of the formula Ip, wherein Het_{A} corresponds to Het-R.85 as defined in table H, and Het_{B} corresponds for each individual compound to one row of table H.
Table 86. Compounds of the formula Ip, wherein Het_{A} corresponds to Het-R.86 as defined in table H, and Het_{B} corresponds for each individual compound to one row of table H.
Table 87. Compounds of the formula Ip, wherein Het_{A} corresponds to Het-R.87 as defined in table H, and Het_{B} corresponds for each individual compound to one row of table H.
Table 88. Compounds of the formula Ip, wherein Het_{A} corresponds to Het-R.88 as defined in table H, and Het_{B} corresponds for each individual compound to one row of table H.
Table 89. Compounds of the formula Ip, wherein Het_{A} corresponds to Het-R.89 as defined in table H, and Het_{B} corresponds for each individual compound to one row of table H.
Table 90. Compounds of the formula Ip, wherein Het_{A} corresponds to Het-R.90 as defined in table H, and Het_{B} corresponds for each individual compound to one row of table H.
Table 91. Compounds of the formula Ip, wherein Het_{A} corresponds to Het-R.91 as defined in table H, and Het_{B} corresponds for each individual compound to one row of table H.
Table 92. Compounds of the formula Ip, wherein Het_{A} corresponds to Het-R.92 as defined in table H, and Het_{B} corresponds for each individual compound to one row of table H.
Table 93. Compounds of the formula Ip, wherein Het_{A} corresponds to Het-R.93 as defined in table H, and Het_{B} corresponds for each individual compound to one row of table H.
Table 94. Compounds of the formula Ip, wherein Het_{A} corresponds to Het-R.94 as defined in table H, and Het_{B} corresponds for each individual compound to one row of table H.
Table 95. Compounds of the formula Ip, wherein Het_{A} corresponds to Het-R.95 as defined in table H, and Het_{B} corresponds for each individual compound to one row of table H.
Table 96. Compounds of the formula Ip, wherein Het_{A} corresponds to Het-R.96 as defined in table H, and Het_{B} corresponds for each individual compound to one row of table H.
Table 97. Compounds of the formula Ip, wherein Het_{A} corresponds to Het-R.97 as defined in table H, and Het_{B} corresponds for each individual compound to one row of table H.
Table 98. Compounds of the formula Ip, wherein Het_{A} corresponds to Het-R.98 as defined in table H, and Het_{B} corresponds for each individual compound to one row of table H.
Table 99. Compounds of the formula Ip, wherein Het_{A} corresponds to Het-R.99 as defined in table H, and Het_{B} corresponds for each individual compound to one row of table H.
Table 100. Compounds of the formula Ip, wherein Het_{A} corresponds to Het-R.100 as defined in table H, and Het_{B} corresponds for each individual compound to one row of table H.
Table 101. Compounds of the formula Ip, wherein Het_{A} corresponds to Het-R.101 as defined in table H, and Het_{B} corresponds for each individual compound to one row of table H.
Table 102. Compounds of the formula Ip, wherein Het_{A} corresponds to Het-R.102 as defined in table H, and Het_{B} corresponds for each individual compound to one row of table H.
Table 103. Compounds of the formula Ip, wherein Het_{A} corresponds to Het-R.103 as defined in table H, and Het_{B} corresponds for each individual compound to one row of table H.
Table 104. Compounds of the formula Ip, wherein Het_{A} corresponds to Het-R.104 as defined in table H, and Het_{B} corresponds for each individual compound to one row of table H.
Table 105. Compounds of the formula Ip, wherein Het_{A} corresponds to Het-R.105 as defined in table H, and Het_{B} corresponds for each individual compound to one row of table H.
Table 106. Compounds of the formula Ip, wherein Het_{A} corresponds to Het-R.106 as defined in table H, and Het_{B} corresponds for each individual compound to one row of table H.
Table 107. Compounds of the formula Ip, wherein Het_{A} corresponds to Het-R.107 as defined in table H, and Het_{B} corresponds for each individual compound to one row of table H.
Table 108. Compounds of the formula Ip, wherein Het_{A} corresponds to Het-R.108 as defined in table H, and Het_{B} corresponds for each individual compound to one row of table H.
Table 109. Compounds of the formula Ip, wherein Het_{A} corresponds to Het-R.109 as defined in table H, and Het_{B} corresponds for each individual compound to one row of table H.
Table 110. Compounds of the formula Ip, wherein Het_{A} corresponds to Het-R.110 as defined in table H, and Het_{B} corresponds for each individual compound to one row of table H.
Table 111. Compounds of the formula Ip, wherein Het_{A} corresponds to Het-R.111 as defined in table H, and Het_{B} corresponds for each individual compound to one row of table H.
Table 112. Compounds of the formula Ip, wherein Het_{A} corresponds to Het-R.112 as defined in table H, and Het_{B} corresponds for each individual compound to one row of table H.
Table 113. Compounds of the formula Ip, wherein Het_{A} corresponds to Het-R.113 as defined in table H, and Het_{B} corresponds for each individual compound to one row of table H.
Table 114. Compounds of the formula Ip, wherein Het_{A} corresponds to Het-R.114 as defined in table H, and Het_{B} corresponds for each individual compound to one row of table H.
Table 115. Compounds of the formula Ip, wherein Het_{A} corresponds to Het-R.115 as defined in table H, and Het_{B} corresponds for each individual compound to one row of table H.
Table 116. Compounds of the formula Ip, wherein Het_{A} corresponds to Het-R.116 as defined in table H, and Het_{B} corresponds for each individual compound to one row of table H.
Table 117. Compounds of the formula Ip, wherein Het_{A} corresponds to Het-R.117 as defined in table H, and Het_{B} corresponds for each individual compound to one row of table H.
Table 118. Compounds of the formula Ip, wherein Het_{A} corresponds to Het-R.118 as defined in table H, and Het_{B} corresponds for each individual compound to one row of table H.
Table 119. Compounds of the formula Ip, wherein Het_{A} corresponds to Het-R.119 as defined in table H, and Het_{B} corresponds for each individual compound to one row of table H.
Table 120. Compounds of the formula Ip, wherein Het_{A} corresponds to Het-R.120 as defined in table H, and Het_{B} corresponds for each individual compound to one row of table H.
Table 121. Compounds of the formula Ip, wherein Het_{A} corresponds to Het-R.121 as defined in table H, and Het_{B} corresponds for each individual compound to one row of table H.
Table 122. Compounds of the formula Ip, wherein Het_{A} corresponds to Het-R.122 as defined in table H, and Het_{B} corresponds for each individual compound to one row of table H.
Table 123. Compounds of the formula Ip, wherein Het_{A} corresponds to Het-R.123 as defined in table H, and Het_{B} corresponds for each individual compound to one row of table H.
Table 124. Compounds of the formula Ip, wherein Het_{A} corresponds to Het-R.124 as defined in table H, and Het_{B} corresponds for each individual compound to one row of table H.
Table 125. Compounds of the formula Ip, wherein Het_{A} corresponds to Het-R.125 as defined in table H, and Het_{B} corresponds for each individual compound to one row of table H.
Table 126. Compounds of the formula Ip, wherein Het_{A} corresponds to Het-R.126 as defined in table H, and Het_{B} corresponds for each individual compound to one row of table H.
Table 127. Compounds of the formula Ip, wherein Het_{A} corresponds to Het-R.127 as defined in table H, and Het_{B} corresponds for each individual compound to one row of table H.
Table 128. Compounds of the formula Ip, wherein Het_{A} corresponds to Het-R.128 as defined in table H, and Het_{B} corresponds for each individual compound to one row of table H.
Table 129. Compounds of the formula Ip, wherein Het_{A} corresponds to Het-R.129 as defined in table H, and Het_{B} corresponds for each individual compound to one row of table H.
Table 130. Compounds of the formula Ip, wherein Het_{A} corresponds to Het-R.130 as defined in table H, and Het_{B} corresponds for each individual compound to one row of table H.
Table 131. Compounds of the formula Ip, wherein Het_{A} corresponds to Het-R.131 as defined in table H, and Het_{B} corresponds for each individual compound to one row of table H.
Table 132. Compounds of the formula Ip, wherein Het_{A} corresponds to Het-R.132 as defined in table H, and Het_{B} corresponds for each individual compound to one row of table H.
Table 133. Compounds of the formula Ip, wherein Het_{A} corresponds to Het-R.133 as defined in table H, and Het_{B} corresponds for each individual compound to one row of table H.
Table 134. Compounds of the formula Ip, wherein Het_{A} corresponds to Het-R.134 as defined in table H, and Het_{B} corresponds for each individual compound to one row of table H.
Table 135. Compounds of the formula Ip, wherein Het_{A} corresponds to Het-R.135 as defined in table H, and Het_{B} corresponds for each individual compound to one row of table H.
Table 136. Compounds of the formula Ip, wherein Het_{A} corresponds to Het-R.136 as defined in table H, and Het_{B} corresponds for each individual compound to one row of table H.
Table 137. Compounds of the formula Ip, wherein Het_{A} corresponds to Het-R.137 as defined in table H, and Het_{B} corresponds for each individual compound to one row of table H.
Table 138. Compounds of the formula Ip, wherein Het_{A} corresponds to Het-R.138 as defined in table H, and Het_{B} corresponds for each individual compound to one row of table H.
Table 139. Compounds of the formula Ip, wherein Het_{A} corresponds to Het-R.139 as defined in table H, and Het_{B} corresponds for each individual compound to one row of table H.
Table 140. Compounds of the formula Ip, wherein Het_{A} corresponds to Het-R.140 as defined in table H, and Het_{B} corresponds for each individual compound to one row of table H.
Table 141. Compounds of the formula Ip, wherein Het_{A} corresponds to Het-R.141 as defined in table H, and Het_{B} corresponds for each individual compound to one row of table H.
Table 142. Compounds of the formula Ip, wherein Het_{A} corresponds to Het-R.142 as defined in table H, and Het_{B} corresponds for each individual compound to one row of table H.
Table 143. Compounds of the formula Ip, wherein Het_{A} corresponds to Het-R.143 as defined in table H, and Het_{B} corresponds for each individual compound to one row of table H.
Table 144. Compounds of the formula Ip, wherein Het_{A} corresponds to Het-R.144 as defined in table H, and Het_{B} corresponds for each individual compound to one row of table H.
Table 145. Compounds of the formula Ip, wherein Het_{A} corresponds to Het-R.145 as defined in table H, and Het_{B} corresponds for each individual compound to one row of table H.
Table 146. Compounds of the formula Ip, wherein Het_{A} corresponds to Het-R.146 as defined in table H, and Het_{B} corresponds for each individual compound to one row of table H.
Table 147. Compounds of the formula Ip, wherein Het_{A} corresponds to Het-R.147 as defined in table H, and Het_{B} corresponds for each individual compound to one row of table H.
Table 148. Compounds of the formula Ip, wherein Het_{A} corresponds to Het-R.148 as defined in table H, and Het_{B} corresponds for each individual compound to one row of table H.
Table 149. Compounds of the formula Ip, wherein Het_{A} corresponds to Het-R.149 as defined in table H, and Het_{B} corresponds for each individual compound to one row of table H.
Table 150. Compounds of the formula Ip, wherein Het_{A} corresponds to Het-R.150 as defined in table H, and Het_{B} corresponds for each individual compound to one row of table H.
Table 151. Compounds of the formula Ip, wherein Het_{A} corresponds to Het-R.151 as defined in table H, and Het_{B} corresponds for each individual compound to one row of table H.
Table 152. Compounds of the formula Ip, wherein Het_{A} corresponds to Het-R.152 as defined in table H, and Het_{B} corresponds for each individual compound to one row of table H.
Table 153. Compounds of the formula Ip, wherein Het_{A} corresponds to Het-R.153 as defined in table H, and Het_{B} corresponds for each individual compound to one row of table H.
Table 154. Compounds of the formula Ip, wherein Het_{A} corresponds to Het-R.154 as defined in table H, and Het_{B} corresponds for each individual compound to one row of table H.
Table 155. Compounds of the formula Ip, wherein Het_{A} corresponds to Het-R.155 as defined in table H, and Het_{B} corresponds for each individual compound to one row of table H.
Table 156. Compounds of the formula Ip, wherein Het_{A} corresponds to Het-R.156 as defined in table H, and Het_{B} corresponds for each individual compound to one row of table H.
Table 157. Compounds of the formula Ip, wherein Het_{A} corresponds to Het-R.157 as defined in table H, and Het_{B} corresponds for each individual compound to one row of table H.
Table 158. Compounds of the formula Ip, wherein Het_{A} corresponds to Het-R.158 as defined in table H, and Het_{B} corresponds for each individual compound to one row of table H.
Table 159. Compounds of the formula Ip, wherein Het_{A} corresponds to Het-R.159 as defined in table H, and Het_{B} corresponds for each individual compound to one row of table H.
Table 160. Compounds of the formula Ip, wherein Het_{A} corresponds to Het-R.160 as defined in table H, and Het_{B} corresponds for each individual compound to one row of table H.
Table 161. Compounds of the formula Ip, wherein Het_{A} corresponds to Het-R.161 as defined in table H, and Het_{B} corresponds for each individual compound to one row of table H.
Table 162. Compounds of the formula Ip, wherein Het_{A} corresponds to Het-R.162 as defined in table H, and Het_{B} corresponds for each individual compound to one row of table H.
Table 163. Compounds of the formula Ip, wherein Het_{A} corresponds to Het-R.163 as defined in table H, and Het_{B} corresponds for each individual compound to one row of table H.
Table 164. Compounds of the formula Ip, wherein Het_{A} corresponds to Het-R.164 as defined in table H, and Het_{B} corresponds for each individual compound to one row of table H.
Table 165. Compounds of the formula Ip, wherein Het_{A} corresponds to Het-R.165 as defined in table H, and Het_{B} corresponds for each individual compound to one row of table H.
Table 166. Compounds of the formula Ip, wherein Het_{A} corresponds to Het-R.166 as defined in table H, and Het_{B} corresponds for each individual compound to one row of table H.
Table 167. Compounds of the formula Ip, wherein Het_{A} corresponds to Het-R.167 as defined in table H, and Het_{B} corresponds for each individual compound to one row of table H.
Table 168. Compounds of the formula Ip, wherein Het_{A} corresponds to Het-R.168 as defined in table H, and Het_{B} corresponds for each individual compound to one row of table H.
Table 169. Compounds of the formula Ip, wherein Het_{A} corresponds to Het-R.169 as defined in table H, and Het_{B} corresponds for each individual compound to one row of table H.
Table 170. Compounds of the formula Ip, wherein Het_{A} corresponds to Het-R.170 as defined in table H, and Het_{B} corresponds for each individual compound to one row of table H.
Table 171. Compounds of the formula Ip, wherein Het_{A} corresponds to Het-R.171 as defined in table H, and Het_{B} corresponds for each individual compound to one row of table H.
Table 172. Compounds of the formula Ip, wherein Het_{A} corresponds to Het-R.172 as defined in table H, and Het_{B} corresponds for each individual compound to one row of table H.
Table 173. Compounds of the formula Ip, wherein Het_{A} corresponds to Het-R.173 as defined in table H, and Het_{B} corresponds for each individual compound to one row of table H.
Table 174. Compounds of the formula Ip, wherein Het_{A} corresponds to Het-R.174 as defined in table H, and Het_{B} corresponds for each individual compound to one row of table H.
Table 175. Compounds of the formula Ip, wherein Het_{A} corresponds to Het-R.175 as defined in table H, and Het_{B} corresponds for each individual compound to one row of table H.
Table 176. Compounds of the formula Ip, wherein Het_{A} corresponds to Het-R.176 as defined in table H, and Het_{B} corresponds for each individual compound to one row of table H.
Table 177. Compounds of the formula Ip, wherein Het_{A} corresponds to Het-R.177 as defined in table H, and Het_{B} corresponds for each individual compound to one row of table H.
Table 178. Compounds of the formula Ip, wherein Het_{A} corresponds to Het-R.178 as defined in table H, and Het_{B} corresponds for each individual compound to one row of table H.
Table 179. Compounds of the formula Ip, wherein Het_{A} corresponds to Het-R.179 as defined in table H, and Het_{B} corresponds for each individual compound to one row of table H.
Table 180. Compounds of the formula Ip, wherein Het_{A} corresponds to Het-R.180 as defined in table H, and Het_{B} corresponds for each individual compound to one row of table H.
Table 181. Compounds of the formula Ip, wherein Het_{A} corresponds to Het-R.181 as defined in table H, and Het_{B} corresponds for each individual compound to one row of table H.
Table 182. Compounds of the formula Ip, wherein Het_{A} corresponds to Het-R.182 as defined in table H, and Het_{B} corresponds for each individual compound to one row of table H.
Table 183. Compounds of the formula Ip, wherein Het_{A} corresponds to Het-R.183 as defined in table H, and Het_{B} corresponds for each individual compound to one row of table H.
Table 184. Compounds of the formula Ip, wherein Het_{A} corresponds to Het-R.184 as defined in table H, and Het_{B} corresponds for each individual compound to one row of table H.
Table 185. Compounds of the formula Ip, wherein Het_{A} corresponds to Het-R.185 as defined in table H, and Het_{B} corresponds for each individual compound to one row of table H.
Table 186. Compounds of the formula Ip, wherein Het_{A} corresponds to Het-R.186 as defined in table H, and Het_{B} corresponds for each individual compound to one row of table H.
Table 187. Compounds of the formula Ip, wherein Het_{A} corresponds to Het-R.187 as defined in table H, and Het_{B} corresponds for each individual compound to one row of table H.
Table 188. Compounds of the formula Ip, wherein Het_{A} corresponds to Het-R.188 as defined in table H, and Het_{B} corresponds for each individual compound to one row of table H.
Table 189. Compounds of the formula Ip, wherein Het_{A} corresponds to Het-R.189 as defined in table H, and Het_{B} corresponds for each individual compound to one row of table H.
Table 190. Compounds of the formula Ip, wherein Het_{A} corresponds to Het-R.190 as defined in table H, and Het_{B} corresponds for each individual compound to one row of table H.
Table 191. Compounds of the formula Ip, wherein Het_{A} corresponds to Het-R.191 as defined in table H, and Het_{B} corresponds for each individual compound to one row of table H.
Table 192. Compounds of the formula Ip, wherein Het_{A} corresponds to Het-R.192 as defined in table H, and Het_{B} corresponds for each individual compound to one row of table H.
Table 193. Compounds of the formula Ip, wherein Het_{A} corresponds to Het-R.193 as defined in table H, and Het_{B} corresponds for each individual compound to one row of table H.
Table 194. Compounds of the formula Ip, wherein Het_{A} corresponds to Het-R.194 as defined in table H, and Het_{B} corresponds for each individual compound to one row of table H.
Table 195. Compounds of the formula Ip, wherein Het_{A} corresponds to Het-R.195 as defined in table H, and Het_{B} corresponds for each individual compound to one row of table H.
Table 196. Compounds of the formula Ip, wherein Het_{A} corresponds to Het-R.196 as defined in table H, and Het_{B} corresponds for each individual compound to one row of table H.
Table 197. Compounds of the formula Ip, wherein Het_{A} corresponds to Het-R.197 as defined in table H, and Het_{B} corresponds for each individual compound to one row of table H.
Table 198. Compounds of the formula Ip, wherein Het_{A} corresponds to Het-R.198 as defined in table H, and Het_{B} corresponds for each individual compound to one row of table H.
Table 199. Compounds of the formula Ip, wherein Het_{A} corresponds to Het-R.199 as defined in table H, and Het_{B} corresponds for each individual compound to one row of table H.
Table 200. Compounds of the formula Ip, wherein Het_{A} corresponds to Het-R.200 as defined in table H, and Het_{B} corresponds for each individual compound to one row of table H.
Table 201. Compounds of the formula Ip, wherein Het_{A} corresponds to Het-R.201 as defined in table H, and Het_{B} corresponds for each individual compound to one row of table H.
Table 202. Compounds of the formula Ip, wherein Het_{A} corresponds to Het-R.202 as defined in table H, and Het_{B} corresponds for each individual compound to one row of table H.
Table 203. Compounds of the formula Ip, wherein Het_{A} corresponds to Het-R.203 as defined in table H, and Het_{B} corresponds for each individual compound to one row of table H.
Table 204. Compounds of the formula Ip, wherein Het_{A} corresponds to Het-R.204 as defined in table H, and Het_{B} corresponds for each individual compound to one row of table H.
Table 205. Compounds of the formula Ip, wherein Het_{A} corresponds to Het-R.205 as defined in table H, and Het_{B} corresponds for each individual compound to one row of table H.
Table 206. Compounds of the formula Ip, wherein Het_{A} corresponds to Het-R.206 as defined in table H, and Het_{B} corresponds for each individual compound to one row of table H.
Table 207. Compounds of the formula Ip, wherein Het_{A} corresponds to Het-R.207 as defined in table H, and Het_{B} corresponds for each individual compound to one row of table H.
Table 208. Compounds of the formula Ip, wherein Het_{A} corresponds to Het-R.208 as defined in table H, and Het_{B} corresponds for each individual compound to one row of table H.
Table 209. Compounds of the formula Ip, wherein Het_{A} corresponds to Het-R.209 as defined in table H, and Het_{B} corresponds for each individual compound to one row of table H.
Table 210. Compounds of the formula Ip, wherein Het_{A} corresponds to Het-R.210 as defined in table H, and Het_{B} corresponds for each individual compound to one row of table H.
Table 211. Compounds of the formula Ip, wherein Het_{A} corresponds to Het-R.211 as defined in table H, and Het_{B} corresponds for each individual compound to one row of table H.
Table 212. Compounds of the formula Ip, wherein Het_{A} corresponds to Het-R.212 as defined in table H, and Het_{B} corresponds for each individual compound to one row of table H.
Table 213. Compounds of the formula Ip, wherein Het_{A} corresponds to Het-R.213 as defined in table H, and Het_{B} corresponds for each individual compound to one row of table H.
Table 214. Compounds of the formula Ip, wherein Het_{A} corresponds to Het-R.214 as defined in table H, and Het_{B} corresponds for each individual compound to one row of table H.
Table 215. Compounds of the formula Ip, wherein Het_{A} corresponds to Het-R.215 as defined in table H, and Het_{B} corresponds for each individual compound to one row of table H.
Table 216. Compounds of the formula Ip, wherein Het_{A} corresponds to Het-R.216 as defined in table H, and Het_{B} corresponds for each individual compound to one row of table H.
Table 217. Compounds of the formula Ip, wherein Het_{A} corresponds to Het-R.217 as defined in table H, and Het_{B} corresponds for each individual compound to one row of table H.
Table 218. Compounds of the formula Ip, wherein Het_{A} corresponds to Het-R.218 as defined in table H, and Het_{B} corresponds for each individual compound to one row of table H.
Table 219. Compounds of the formula Ip, wherein Het_{A} corresponds to Het-R.219 as defined in table H, and Het_{B} corresponds for each individual compound to one row of table H.
Table 220. Compounds of the formula Ip, wherein Het_{A} corresponds to Het-R.220 as defined in table H, and Het_{B} corresponds for each individual compound to one row of table H.
Table 221. Compounds of the formula Ip, wherein Het_{A} corresponds to Het-R.221 as defined in table H, and Het_{B} corresponds for each individual compound to one row of table H.
Table 222. Compounds of the formula Ip, wherein Het_{A} corresponds to Het-R.222 as defined in table H, and Het_{B} corresponds for each individual compound to one row of table H.
Table 223. Compounds of the formula Ip, wherein Het_{A} corresponds to Het-R.223 as defined in table H, and Het_{B} corresponds for each individual compound to one row of table H.
Table 224. Compounds of the formula Ip, wherein Het_{A} corresponds to Het-R.224 as defined in table H, and Het_{B} corresponds for each individual compound to one row of table H.
Table 225. Compounds of the formula Ip, wherein Het_{A} corresponds to Het-R.225 as defined in table H, and Het_{B} corresponds for each individual compound to one row of table H.
Table 226. Compounds of the formula Ip, wherein Het_{A} corresponds to Het-R.226 as defined in table H, and Het_{B} corresponds for each individual compound to one row of table H.
Table 227. Compounds of the formula Ip, wherein Het_{A} corresponds to Het-R.227 as defined in table H, and Het_{B} corresponds for each individual compound to one row of table H.
Table 228. Compounds of the formula Ip, wherein Het_{A} corresponds to Het-R.228 as defined in table H, and Het_{B} corresponds for each individual compound to one row of table H.
Table 229. Compounds of the formula Ip, wherein Het_{A} corresponds to Het-R.229 as defined in table H, and Het_{B} corresponds for each individual compound to one row of table H.
Table 230. Compounds of the formula Ip, wherein Het_{A} corresponds to Het-R.230 as defined in table H, and Het_{B} corresponds for each individual compound to one row of table H.
Table 231. Compounds of the formula Ip, wherein Het_{A} corresponds to Het-R.231 as defined in table H, and Het_{B} corresponds for each individual compound to one row of table H.
Table 232. Compounds of the formula Ip, wherein Het_{A} corresponds to Het-R.232 as defined in table H, and Het_{B} corresponds for each individual compound to one row of table H.

Examples of preferred compounds I are also the compounds of formula I, wherein R¹, R², R³ are hydrogen, A is a radical A² with R^{4a}, R^{4b}, R^{4c} and R^{4d} being hydrogen, X is O and R⁶ is hydrogen, and wherein Het_{A} and Het_{B} are as defined in the tables 1 to 232.

Examples of preferred compounds I are also the compounds of formula I, wherein R¹, R², R³ are hydrogen, A is a radical A² with R^{4a}, R^{4b}, R^{4c} and R^{4d} being hydrogen, X is NH and R⁶ is hydrogen, and wherein Het_{A} and Het_{B} are as defined in the tables 1 to 232.

Examples of preferred compounds I are also the compounds of formula I, wherein R¹, R², R³ are hydrogen, A is a radical A² with R^{4a}, R^{4b}, R^{4c} and R^{4d} being hydrogen, X is NCH₃ and R⁶ is hydrogen, and wherein Het_{A} and Het_{B} are as defined in the tables 1 to 232.

Examples of preferred compounds I are also the compounds of formula I, wherein R¹, R², R³ are hydrogen, A is a radical A² with R^{4a}, R^{4b}, R^{4c} and R^{4d} being hydrogen, X is NC(O)CH₃ and R⁶ is hydrogen, and wherein Het_{A} and Het_{B} are as defined in the tables 1 to 232.

### Compounds of the General Formula II and the Salts Thereof

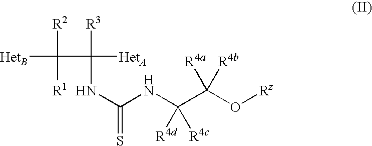

wherein Het_{A}, Het_{B}, R¹, R², R³, R^{4a}, R^{4b}, R^{4c} and R^{4d} are as defined above and wherein R^{z} is hydrogen or acetyl, are new and thus form part of the invention.

As regards the pesticidal activity of the compounds of general formula II, preference is given to those compounds II wherein the variables Het_{A}, Het_{B}, R¹, R², R³, R^{4a}, R^{4b}, R^{4c} and R^{4d} have independently of each other or more preferably in combination the meanings mentioned above as being preferred as for compounds of formula I.

Particular preference is also given to those compounds II, wherein

R¹, R², R³ are hydrogen;
R^{4a}, R^{4b}, R^{4c}, R^{4d} are hydrogen; and
R^{z} is hydrogen.

Particular preference is also given to those compounds II, wherein

R¹, R², R³ are hydrogen;
R^{4a}, R^{4b}, R^{4c}, R^{4d} are hydrogen; and
R^{z} is acetyl.

Examples of preferred compounds II, wherein R¹, R², R³ are hydrogen, R^{4a}, R^{4b}, R^{4c} and R^{4d} are hydrogen, and R^{z} is hydrogen are described in the following tables 233 to 464 (hereinafter also referred to as compounds IIp).

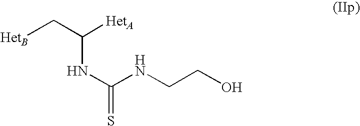

Table 233. Compounds of the formula IIp, wherein Het_{A} corresponds to Het-R.1 as defined in table H, and Het_{B} corresponds for each individual compound to one row of table H.
Table 234. Compounds of the formula IIp, wherein Het_{A} corresponds to Het-R.2 as defined in table H, and Het_{B} corresponds for each individual compound to one row of table H.
Table 235. Compounds of the formula IIp, wherein Het_{A} corresponds to Het-R.3 as defined in table H, and Het_{B} corresponds for each individual compound to one row of table H.
Table 236. Compounds of the formula IIp, wherein Het_{A} corresponds to Het-R.4 as defined in table H, and Het_{B} corresponds for each individual compound to one row of table H.
Table 237. Compounds of the formula IIp, wherein Het_{A} corresponds to Het-R.5 as defined in table H, and Het_{B} corresponds for each individual compound to one row of table H.
Table 238. Compounds of the formula IIp, wherein Het_{A} corresponds to Het-R.6 as defined in table H, and Het_{B} corresponds for each individual compound to one row of table H.
Table 239. Compounds of the formula IIp, wherein Het_{A} corresponds to Het-R.7 as defined in table H, and Het_{B} corresponds for each individual compound to one row of table H.
Table 240. Compounds of the formula IIp, wherein Het_{A} corresponds to Het-R.8 as defined in table H, and Het_{B} corresponds for each individual compound to one row of table H.
Table 241. Compounds of the formula IIp, wherein Het_{A} corresponds to Het-R.9 as defined in table H, and Het_{B} corresponds for each individual compound to one row of table H.
Table 242. Compounds of the formula IIp, wherein Het_{A} corresponds to Het-R.10 as defined in table H, and Het_{B} corresponds for each individual compound to one row of table H.
Table 243. Compounds of the formula IIp, wherein Het_{A} corresponds to Het-R.11 as defined in table H, and Het_{B} corresponds for each individual compound to one row of table H.
Table 244. Compounds of the formula IIp, wherein Het_{A} corresponds to Het-R.12 as defined in table H, and Het_{B} corresponds for each individual compound to one row of table H.
Table 245. Compounds of the formula IIp, wherein Het_{A} corresponds to Het-R.13 as defined in table H, and Het_{B} corresponds for each individual compound to one row of table H.
Table 246. Compounds of the formula IIp, wherein Het_{A} corresponds to Het-R.14 as defined in table H, and Het_{B} corresponds for each individual compound to one row of table H.
Table 247. Compounds of the formula IIp, wherein Het_{A} corresponds to Het-R.15 as defined in table H, and Het_{B} corresponds for each individual compound to one row of table H.
Table 248. Compounds of the formula IIp, wherein Het_{A} corresponds to Het-R.16 as defined in table H, and Het_{B} corresponds for each individual compound to one row of table H.
Table 249. Compounds of the formula IIp, wherein Het_{A} corresponds to Het-R.17 as defined in table H, and Het_{B} corresponds for each individual compound to one row of table H.
Table 250. Compounds of the formula IIp, wherein Het_{A} corresponds to Het-R.18 as defined in table H, and Het_{B} corresponds for each individual compound to one row of table H.
Table 251. Compounds of the formula IIp, wherein Het_{A} corresponds to Het-R.19 as defined in table H, and Het_{B} corresponds for each individual compound to one row of table H.
Table 252. Compounds of the formula IIp, wherein Het_{A} corresponds to Het-R.20 as defined in table H, and Het_{B} corresponds for each individual compound to one row of table H.
Table 253. Compounds of the formula IIp, wherein Het_{A} corresponds to Het-R.21 as defined in table H, and Het_{B} corresponds for each individual compound to one row of table H.
Table 254. Compounds of the formula IIp, wherein Het_{A} corresponds to Het-R.22 as defined in table H, and Het_{B} corresponds for each individual compound to one row of table H.
Table 255. Compounds of the formula IIp, wherein Het_{A} corresponds to Het-R.23 as defined in table H, and Het_{B} corresponds for each individual compound to one row of table H.
Table 256. Compounds of the formula IIp, wherein Het_{A} corresponds to Het-R.24 as defined in table H, and Het_{B} corresponds for each individual compound to one row of table H.
Table 257. Compounds of the formula IIp, wherein Het_{A} corresponds to Het-R.25 as defined in table H, and Het_{B} corresponds for each individual compound to one row of table H.
Table 258. Compounds of the formula IIp, wherein Het_{A} corresponds to Het-R.26 as defined in table H, and Het_{B} corresponds for each individual compound to one row of table H.
Table 259. Compounds of the formula IIp, wherein Het_{A} corresponds to Het-R.27 as defined in table H, and Het_{B} corresponds for each individual compound to one row of table H.
Table 260. Compounds of the formula IIp, wherein Het_{A} corresponds to Het-R.28 as defined in table H, and Het_{B} corresponds for each individual compound to one row of table H.
Table 261. Compounds of the formula IIp, wherein Het_{A} corresponds to Het-R.29 as defined in table H, and Het_{B} corresponds for each individual compound to one row of table H.
Table 262. Compounds of the formula IIp, wherein Het_{A} corresponds to Het-R.30 as defined in table H, and Het_{B} corresponds for each individual compound to one row of table H.
Table 263. Compounds of the formula IIp, wherein Het_{A} corresponds to Het-R.31 as defined in table H, and Het_{B} corresponds for each individual compound to one row of table H.
Table 264. Compounds of the formula IIp, wherein Het_{A} corresponds to Het-R.32 as defined in table H, and Het_{B} corresponds for each individual compound to one row of table H.
Table 265. Compounds of the formula IIp, wherein Het_{A} corresponds to Het-R.33 as defined in table H, and Het_{B} corresponds for each individual compound to one row of table H.
Table 266. Compounds of the formula IIp, wherein Het_{A} corresponds to Het-R.34 as defined in table H, and Het_{B} corresponds for each individual compound to one row of table H.
Table 267. Compounds of the formula IIp, wherein Het_{A} corresponds to Het-R.35 as defined in table H, and Het_{B} corresponds for each individual compound to one row of table H.
Table 268. Compounds of the formula IIp, wherein Het_{A} corresponds to Het-R.36 as defined in table H, and Het_{B} corresponds for each individual compound to one row of table H.
Table 269. Compounds of the formula IIp, wherein Het_{A} corresponds to Het-R.37 as defined in table H, and Het_{B} corresponds for each individual compound to one row of table H.
Table 270. Compounds of the formula IIp, wherein Het_{A} corresponds to Het-R.38 as defined in table H, and Het_{B} corresponds for each individual compound to one row of table H.
Table 271. Compounds of the formula IIp, wherein Het_{A} corresponds to Het-R.39 as defined in table H, and Het_{B} corresponds for each individual compound to one row of table H.
Table 272. Compounds of the formula IIp, wherein Het_{A} corresponds to Het-R.40 as defined in table H, and Het_{B} corresponds for each individual compound to one row of table H.
Table 273. Compounds of the formula IIp, wherein Het_{A} corresponds to Het-R.41 as defined in table H, and Het_{B} corresponds for each individual compound to one row of table H.
Table 274. Compounds of the formula IIp, wherein Het_{A} corresponds to Het-R.42 as defined in table H, and Het_{B} corresponds for each individual compound to one row of table H.
Table 275. Compounds of the formula IIp, wherein Het_{A} corresponds to Het-R.43 as defined in table H, and Het_{B} corresponds for each individual compound to one row of table H.
Table 276. Compounds of the formula IIp, wherein Het_{A} corresponds to Het-R.44 as defined in table H, and Het_{B} corresponds for each individual compound to one row of table H.
Table 277. Compounds of the formula IIp, wherein Het_{A} corresponds to Het-R.45 as defined in table H, and Het_{B} corresponds for each individual compound to one row of table H.
Table 278. Compounds of the formula IIp, wherein Het_{A} corresponds to Het-R.46 as defined in table H, and Het_{B} corresponds for each individual compound to one row of table H.
Table 279. Compounds of the formula IIp, wherein Het_{A} corresponds to Het-R.47 as defined in table H, and Het_{B} corresponds for each individual compound to one row of table H.
Table 280. Compounds of the formula IIp, wherein Het_{A} corresponds to Het-R.48 as defined in table H, and Het_{B} corresponds for each individual compound to one row of table H.
Table 281. Compounds of the formula IIp, wherein Het_{A} corresponds to Het-R.49 as defined in table H, and Het_{B} corresponds for each individual compound to one row of table H.
Table 282. Compounds of the formula IIp, wherein Het_{A} corresponds to Het-R.50 as defined in table H, and Het_{B} corresponds for each individual compound to one row of table H.
Table 283. Compounds of the formula IIp, wherein Het_{A} corresponds to Het-R.51 as defined in table H, and Het_{B} corresponds for each individual compound to one row of table H.
Table 284. Compounds of the formula IIp, wherein Het_{A} corresponds to Het-R.52 as defined in table H, and Het_{B} corresponds for each individual compound to one row of table H.
Table 285. Compounds of the formula IIp, wherein Het_{A} corresponds to Het-R.53 as defined in table H, and Het_{B} corresponds for each individual compound to one row of table H.
Table 286. Compounds of the formula IIp, wherein Het_{A} corresponds to Het-R.54 as defined in table H, and Het_{B} corresponds for each individual compound to one row of table H.
Table 287. Compounds of the formula IIp, wherein Het_{A} corresponds to Het-R.55 as defined in table H, and Het_{B} corresponds for each individual compound to one row of table H.
Table 288. Compounds of the formula IIp, wherein Het_{A} corresponds to Het-R.56 as defined in table H, and Het_{B} corresponds for each individual compound to one row of table H.
Table 289. Compounds of the formula IIp, wherein Het_{A} corresponds to Het-R.57 as defined in table H, and Het_{B} corresponds for each individual compound to one row of table H.
Table 290. Compounds of the formula IIp, wherein Het_{A} corresponds to Het-R.58 as defined in table H, and Het_{B} corresponds for each individual compound to one row of table H.
Table 291. Compounds of the formula IIp, wherein Het_{A} corresponds to Het-R.59 as defined in table H, and Het_{B} corresponds for each individual compound to one row of table H.
Table 292. Compounds of the formula IIp, wherein Het_{A} corresponds to Het-R.60 as defined in table H, and Het_{B} corresponds for each individual compound to one row of table H.
Table 293. Compounds of the formula IIp, wherein Het_{A} corresponds to Het-R.61 as defined in table H, and Het_{B} corresponds for each individual compound to one row of table H.
Table 294. Compounds of the formula IIp, wherein Het_{A} corresponds to Het-R.62 as defined in table H, and Het_{B} corresponds for each individual compound to one row of table H.
Table 295. Compounds of the formula IIp, wherein Het_{A} corresponds to Het-R.63 as defined in table H, and Het_{B} corresponds for each individual compound to one row of table H.
Table 296. Compounds of the formula IIp, wherein Het_{A} corresponds to Het-R.64 as defined in table H, and Het_{B} corresponds for each individual compound to one row of table H.
Table 297. Compounds of the formula IIp, wherein Het_{A} corresponds to Het-R.65 as defined in table H, and Het_{B} corresponds for each individual compound to one row of table H.
Table 298. Compounds of the formula IIp, wherein Het_{A} corresponds to Het-R.66 as defined in table H, and Het_{B} corresponds for each individual compound to one row of table H.
Table 299. Compounds of the formula IIp, wherein Het_{A} corresponds to Het-R.67 as defined in table H, and Het_{B} corresponds for each individual compound to one row of table H.
Table 300. Compounds of the formula IIp, wherein Het_{A} corresponds to Het-R.68 as defined in table H, and Het_{B} corresponds for each individual compound to one row of table H.
Table 301. Compounds of the formula IIp, wherein Het_{A} corresponds to Het-R.69 as defined in table H, and Het_{B} corresponds for each individual compound to one row of table H.
Table 302. Compounds of the formula IIp, wherein Het_{A} corresponds to Het-R.70 as defined in table H, and Het_{B} corresponds for each individual compound to one row of table H.
Table 303. Compounds of the formula IIp, wherein Het_{A} corresponds to Het-R.71 as defined in table H, and Het_{B} corresponds for each individual compound to one row of table H.
Table 304. Compounds of the formula IIp, wherein Het_{A} corresponds to Het-R.72 as defined in table H, and Het_{B} corresponds for each individual compound to one row of table H.
Table 305. Compounds of the formula IIp, wherein Het_{A} corresponds to Het-R.73 as defined in table H, and Het_{B} corresponds for each individual compound to one row of table H.
Table 306. Compounds of the formula IIp, wherein Het_{A} corresponds to Het-R.74 as defined in table H, and Het_{B} corresponds for each individual compound to one row of table H.
Table 307. Compounds of the formula IIp, wherein Het_{A} corresponds to Het-R.75 as defined in table H, and Het_{B} corresponds for each individual compound to one row of table H.
Table 308. Compounds of the formula IIp, wherein Het_{A} corresponds to Het-R.76 as defined in table H, and Het_{B} corresponds for each individual compound to one row of table H.
Table 309. Compounds of the formula IIp, wherein Het_{A} corresponds to Het-R.77 as defined in table H, and Het_{B} corresponds for each individual compound to one row of table H.
Table 310. Compounds of the formula IIp, wherein Het_{A} corresponds to Het-R.78 as defined in table H, and Het_{B} corresponds for each individual compound to one row of table H.
Table 311. Compounds of the formula IIp, wherein Het_{A} corresponds to Het-R.79 as defined in table H, and Het_{B} corresponds for each individual compound to one row of table H.
Table 312. Compounds of the formula IIp, wherein Het_{A} corresponds to Het-R.80 as defined in table H, and Het_{B} corresponds for each individual compound to one row of table H.
Table 313. Compounds of the formula IIp, wherein Het_{A} corresponds to Het-R.81 as defined in table H, and Het_{B} corresponds for each individual compound to one row of table H.
Table 314. Compounds of the formula IIp, wherein Het_{A} corresponds to Het-R.82 as defined in table H, and Het_{B} corresponds for each individual compound to one row of table H.
Table 315. Compounds of the formula IIp, wherein Het_{A} corresponds to Het-R.83 as defined in table H, and Het_{B} corresponds for each individual compound to one row of table H.
Table 316. Compounds of the formula IIp, wherein Het_{A} corresponds to Het-R.84 as defined in table H, and Het_{B} corresponds for each individual compound to one row of table H.
Table 317. Compounds of the formula IIp, wherein Het_{A} corresponds to Het-R.85 as defined in table H, and Het_{B} corresponds for each individual compound to one row of table H.
Table 318. Compounds of the formula IIp, wherein Het_{A} corresponds to Het-R.86 as defined in table H, and Het_{B} corresponds for each individual compound to one row of table H.
Table 319. Compounds of the formula IIp, wherein Het_{A} corresponds to Het-R.87 as defined in table H, and Het_{B} corresponds for each individual compound to one row of table H.
Table 320. Compounds of the formula IIp, wherein Het_{A} corresponds to Het-R.88 as defined in table H, and Het_{B} corresponds for each individual compound to one row of table H.
Table 321. Compounds of the formula IIp, wherein Het_{A} corresponds to Het-R.89 as defined in table H, and Het_{B} corresponds for each individual compound to one row of table H.
Table 322. Compounds of the formula IIp, wherein Het_{A} corresponds to Het-R.90 as defined in table H, and Het_{B} corresponds for each individual compound to one row of table H.
Table 323. Compounds of the formula IIp, wherein Het_{A} corresponds to Het-R.91 as defined in table H, and Het_{B} corresponds for each individual compound to one row of table H.
Table 324. Compounds of the formula IIp, wherein Het_{A} corresponds to Het-R.92 as defined in table H, and Het_{B} corresponds for each individual compound to one row of table H.
Table 325. Compounds of the formula IIp, wherein Het_{A} corresponds to Het-R.93 as defined in table H, and Het_{B} corresponds for each individual compound to one row of table H.
Table 326. Compounds of the formula IIp, wherein Het_{A} corresponds to Het-R.94 as defined in table H, and Het_{B} corresponds for each individual compound to one row of table H.
Table 327. Compounds of the formula IIp, wherein Het_{A} corresponds to Het-R.95 as defined in table H, and Het_{B} corresponds for each individual compound to one row of table H.
Table 328. Compounds of the formula IIp, wherein Het_{A} corresponds to Het-R.96 as defined in table H, and Het_{B} corresponds for each individual compound to one row of table H.
Table 329. Compounds of the formula IIp, wherein Het_{A} corresponds to Het-R.97 as defined in table H, and Het_{B} corresponds for each individual compound to one row of table H.
Table 330. Compounds of the formula IIp, wherein Het_{A} corresponds to Het-R.98 as defined in table H, and Het_{B} corresponds for each individual compound to one row of table H.
Table 331. Compounds of the formula IIp, wherein Het_{A} corresponds to Het-R.99 as defined in table H, and Het_{B} corresponds for each individual compound to one row of table H.
Table 332. Compounds of the formula IIp, wherein Het_{A} corresponds to Het-R.100 as defined in table H, and Het_{B} corresponds for each individual compound to one row of table H.
Table 333. Compounds of the formula IIp, wherein Het_{A} corresponds to Het-R.101 as defined in table H, and Het_{B} corresponds for each individual compound to one row of table H.
Table 334. Compounds of the formula IIp, wherein Het_{A} corresponds to Het-R.102 as defined in table H, and Het_{B} corresponds for each individual compound to one row of table H.
Table 335. Compounds of the formula IIp, wherein Het_{A} corresponds to Het-R.103 as defined in table H, and Het_{B} corresponds for each individual compound to one row of table H.
Table 336. Compounds of the formula IIp, wherein Het_{A} corresponds to Het-R.104 as defined in table H, and Het_{B} corresponds for each individual compound to one row of table H.
Table 337. Compounds of the formula IIp, wherein Het_{A} corresponds to Het-R.105 as defined in table H, and Het_{B} corresponds for each individual compound to one row of table H.
Table 338. Compounds of the formula IIp, wherein Het_{A} corresponds to Het-R.106 as defined in table H, and Het_{B} corresponds for each individual compound to one row of table H.
Table 339. Compounds of the formula IIp, wherein Het_{A} corresponds to Het-R.107 as defined in table H, and Het_{B} corresponds for each individual compound to one row of table H.
Table 340. Compounds of the formula IIp, wherein Het_{A} corresponds to Het-R.108 as defined in table H, and Het_{B} corresponds for each individual compound to one row of table H.
Table 341. Compounds of the formula IIp, wherein Het_{A} corresponds to Het-R.109 as defined in table H, and Het_{B} corresponds for each individual compound to one row of table H.
Table 342. Compounds of the formula IIp, wherein Het_{A} corresponds to Het-R.110 as defined in table H, and Het_{B} corresponds for each individual compound to one row of table H.
Table 343. Compounds of the formula IIp, wherein Het_{A} corresponds to Het-R.111 as defined in table H, and Het_{B} corresponds for each individual compound to one row of table H.
Table 344. Compounds of the formula IIp, wherein Het_{A} corresponds to Het-R.112 as defined in table H, and Het_{B} corresponds for each individual compound to one row of table H.
Table 345. Compounds of the formula IIp, wherein Het_{A} corresponds to Het-R.113 as defined in table H, and Het_{B} corresponds for each individual compound to one row of table H.
Table 346. Compounds of the formula IIp, wherein Het_{A} corresponds to Het-R.114 as defined in table H, and Het_{B} corresponds for each individual compound to one row of table H.
Table 347. Compounds of the formula IIp, wherein Het_{A} corresponds to Het-R.115 as defined in table H, and Het_{B} corresponds for each individual compound to one row of table H.
Table 348. Compounds of the formula IIp, wherein Het_{A} corresponds to Het-R.116 as defined in table H, and Het_{B} corresponds for each individual compound to one row of table H.
Table 349. Compounds of the formula IIp, wherein Het_{A} corresponds to Het-R.117 as defined in table H, and Het_{B} corresponds for each individual compound to one row of table H.
Table 350. Compounds of the formula IIp, wherein Het_{A} corresponds to Het-R.118 as defined in table H, and Het_{B} corresponds for each individual compound to one row of table H.
Table 351. Compounds of the formula IIp, wherein Het_{A} corresponds to Het-R.119 as defined in table H, and Het_{B} corresponds for each individual compound to one row of table H.
Table 352. Compounds of the formula IIp, wherein Het_{A} corresponds to Het-R.120 as defined in table H, and Het_{B} corresponds for each individual compound to one row of table H.
Table 353. Compounds of the formula IIp, wherein Het_{A} corresponds to Het-R.121 as defined in table H, and Het_{B} corresponds for each individual compound to one row of table H.
Table 354. Compounds of the formula IIp, wherein Het_{A} corresponds to Het-R.122 as defined in table H, and Het_{B} corresponds for each individual compound to one row of table H.
Table 355. Compounds of the formula IIp, wherein Het_{A} corresponds to Het-R.123 as defined in table H, and Het_{B} corresponds for each individual compound to one row of table H.
Table 356. Compounds of the formula IIp, wherein Het_{A} corresponds to Het-R.124 as defined in table H, and Het_{B} corresponds for each individual compound to one row of table H.
Table 357. Compounds of the formula IIp, wherein Het_{A} corresponds to Het-R.125 as defined in table H, and Het_{B} corresponds for each individual compound to one row of table H.
Table 358. Compounds of the formula IIp, wherein Het_{A} corresponds to Het-R.126 as defined in table H, and Het_{B} corresponds for each individual compound to one row of table H.
Table 359. Compounds of the formula IIp, wherein Het_{A} corresponds to Het-R.127 as defined in table H, and Het_{B} corresponds for each individual compound to one row of table H.
Table 360. Compounds of the formula IIp, wherein Het_{A} corresponds to Het-R.128 as defined in table H, and Het_{B} corresponds for each individual compound to one row of table H.
Table 361. Compounds of the formula IIp, wherein Het_{A} corresponds to Het-R.129 as defined in table H, and Het_{B} corresponds for each individual compound to one row of table H.
Table 362. Compounds of the formula IIp, wherein Het_{A} corresponds to Het-R.130 as defined in table H, and Het_{B} corresponds for each individual compound to one row of table H.
Table 363. Compounds of the formula IIp, wherein Het_{A} corresponds to Het-R.131 as defined in table H, and Het_{B} corresponds for each individual compound to one row of table H.
Table 364. Compounds of the formula IIp, wherein Het_{A} corresponds to Het-R.132 as defined in table H, and Het_{B} corresponds for each individual compound to one row of table H.
Table 365. Compounds of the formula IIp, wherein Het_{A} corresponds to Het-R.133 as defined in table H, and Het_{B} corresponds for each individual compound to one row of table H.
Table 366. Compounds of the formula IIp, wherein Het_{A} corresponds to Het-R.134 as defined in table H, and Het_{B} corresponds for each individual compound to one row of table H.
Table 367. Compounds of the formula IIp, wherein Het_{A} corresponds to Het-R.135 as defined in table H, and Het_{B} corresponds for each individual compound to one row of table H.
Table 368. Compounds of the formula IIp, wherein Het_{A} corresponds to Het-R.136 as defined in table H, and Het_{B} corresponds for each individual compound to one row of table H.
Table 369. Compounds of the formula IIp, wherein Het_{A} corresponds to Het-R.137 as defined in table H, and Het_{B} corresponds for each individual compound to one row of table H.
Table 370. Compounds of the formula IIp, wherein Het_{A} corresponds to Het-R.138 as defined in table H, and Het_{B} corresponds for each individual compound to one row of table H.
Table 371. Compounds of the formula IIp, wherein Het_{A} corresponds to Het-R.139 as defined in table H, and Het_{B} corresponds for each individual compound to one row of table H.
Table 372. Compounds of the formula IIp, wherein Het_{A} corresponds to Het-R.140 as defined in table H, and Het_{B} corresponds for each individual compound to one row of table H.
Table 373. Compounds of the formula IIp, wherein Het_{A} corresponds to Het-R.141 as defined in table H, and Het_{B} corresponds for each individual compound to one row of table H.
Table 374. Compounds of the formula IIp, wherein Het_{A} corresponds to Het-R.142 as defined in table H, and Het_{B} corresponds for each individual compound to one row of table H.
Table 375. Compounds of the formula IIp, wherein Het_{A} corresponds to Het-R.143 as defined in table H, and Het_{B} corresponds for each individual compound to one row of table H.
Table 376. Compounds of the formula IIp, wherein Het_{A} corresponds to Het-R.144 as defined in table H, and Het_{B} corresponds for each individual compound to one row of table H.
Table 377. Compounds of the formula IIp, wherein Het_{A} corresponds to Het-R.145 as defined in table H, and Het_{B} corresponds for each individual compound to one row of table H.
Table 378. Compounds of the formula IIp, wherein Het_{A} corresponds to Het-R.146 as defined in table H, and Het_{B} corresponds for each individual compound to one row of table H.
Table 379. Compounds of the formula IIp, wherein Het_{A} corresponds to Het-R.147 as defined in table H, and Het_{B} corresponds for each individual compound to one row of table H.
Table 380. Compounds of the formula IIp, wherein Het_{A} corresponds to Het-R.148 as defined in table H, and Het_{B} corresponds for each individual compound to one row of table H.
Table 381. Compounds of the formula IIp, wherein Het_{A} corresponds to Het-R.149 as defined in table H, and Het_{B} corresponds for each individual compound to one row of table H.
Table 382. Compounds of the formula IIp, wherein Het_{A} corresponds to Het-R.150 as defined in table H, and Het_{B} corresponds for each individual compound to one row of table H.
Table 383. Compounds of the formula IIp, wherein Het_{A} corresponds to Het-R.151 as defined in table H, and Het_{B} corresponds for each individual compound to one row of table H.
Table 384. Compounds of the formula IIp, wherein Het_{A} corresponds to Het-R.152 as defined in table H, and Het_{B} corresponds for each individual compound to one row of table H.
Table 385. Compounds of the formula IIp, wherein Het_{A} corresponds to Het-R.153 as defined in table H, and Het_{B} corresponds for each individual compound to one row of table H.
Table 386. Compounds of the formula IIp, wherein Het_{A} corresponds to Het-R.154 as defined in table H, and Het_{B} corresponds for each individual compound to one row of table H.
Table 387. Compounds of the formula IIp, wherein Het_{A} corresponds to Het-R.155 as defined in table H, and Het_{B} corresponds for each individual compound to one row of table H.
Table 388. Compounds of the formula IIp, wherein Het_{A} corresponds to Het-R.156 as defined in table H, and Het_{B} corresponds for each individual compound to one row of table H.
Table 389. Compounds of the formula IIp, wherein Het_{A} corresponds to Het-R.157 as defined in table H, and Het_{B} corresponds for each individual compound to one row of table H.
Table 390. Compounds of the formula IIp, wherein Het_{A} corresponds to Het-R.158 as defined in table H, and Het_{B} corresponds for each individual compound to one row of table H.
Table 391. Compounds of the formula IIp, wherein Het_{A} corresponds to Het-R.159 as defined in table H, and Het_{B} corresponds for each individual compound to one row of table H.
Table 392. Compounds of the formula IIp, wherein Het_{A} corresponds to Het-R.160 as defined in table H, and Het_{B} corresponds for each individual compound to one row of table H.
Table 393. Compounds of the formula IIp, wherein Het_{A} corresponds to Het-R.161 as defined in table H, and Het_{B} corresponds for each individual compound to one row of table H.
Table 394. Compounds of the formula IIp, wherein Het_{A} corresponds to Het-R.162 as defined in table H, and Het_{B} corresponds for each individual compound to one row of table H.
Table 395. Compounds of the formula IIp, wherein Het_{A} corresponds to Het-R.163 as defined in table H, and Het_{B} corresponds for each individual compound to one row of table H.
Table 396. Compounds of the formula IIp, wherein Het_{A} corresponds to Het-R.164 as defined in table H, and Het_{B} corresponds for each individual compound to one row of table H.
Table 397. Compounds of the formula IIp, wherein Het_{A} corresponds to Het-R.165 as defined in table H, and Het_{B} corresponds for each individual compound to one row of table H.
Table 398. Compounds of the formula IIp, wherein Het_{A} corresponds to Het-R.166 as defined in table H, and Het_{B} corresponds for each individual compound to one row of table H.
Table 399. Compounds of the formula IIp, wherein Het_{A} corresponds to Het-R.167 as defined in table H, and Het_{B} corresponds for each individual compound to one row of table H.
Table 400. Compounds of the formula IIp, wherein Het_{A} corresponds to Het-R.168 as defined in table H, and Het_{B} corresponds for each individual compound to one row of table H.
Table 401. Compounds of the formula IIp, wherein Het_{A} corresponds to Het-R.169 as defined in table H, and Het_{B} corresponds for each individual compound to one row of table H.
Table 402. Compounds of the formula IIp, wherein Het_{A} corresponds to Het-R.170 as defined in table H, and Het_{B} corresponds for each individual compound to one row of table H.
Table 403. Compounds of the formula IIp, wherein Het_{A} corresponds to Het-R.171 as defined in table H, and Het_{B} corresponds for each individual compound to one row of table H.
Table 404. Compounds of the formula IIp, wherein Het_{A} corresponds to Het-R.172 as defined in table H, and Het_{B} corresponds for each individual compound to one row of table H.
Table 405. Compounds of the formula IIp, wherein Het_{A} corresponds to Het-R.173 as defined in table H, and Het_{B} corresponds for each individual compound to one row of table H.
Table 406. Compounds of the formula IIp, wherein Het_{A} corresponds to Het-R.174 as defined in table H, and Het_{B} corresponds for each individual compound to one row of table H.
Table 407. Compounds of the formula IIp, wherein Het_{A} corresponds to Het-R.175 as defined in table H, and Het_{B} corresponds for each individual compound to one row of table H.
Table 408. Compounds of the formula IIp, wherein Het_{A} corresponds to Het-R.176 as defined in table H, and Het_{B} corresponds for each individual compound to one row of table H.
Table 409. Compounds of the formula IIp, wherein Het_{A} corresponds to Het-R.177 as defined in table H, and Het_{B} corresponds for each individual compound to one row of table H.
Table 410. Compounds of the formula IIp, wherein Het_{A} corresponds to Het-R.178 as defined in table H, and Het_{B} corresponds for each individual compound to one row of table H.
Table 411. Compounds of the formula IIp, wherein Het_{A} corresponds to Het-R.179 as defined in table H, and Het_{B} corresponds for each individual compound to one row of table H.
Table 412. Compounds of the formula IIp, wherein Het_{A} corresponds to Het-R.180 as defined in table H, and Het_{B} corresponds for each individual compound to one row of table H.
Table 413. Compounds of the formula IIp, wherein Het_{A} corresponds to Het-R.181 as defined in table H, and Het_{B} corresponds for each individual compound to one row of table H.
Table 414. Compounds of the formula IIp, wherein Het_{A} corresponds to Het-R.182 as defined in table H, and Het_{B} corresponds for each individual compound to one row of table H.
Table 415. Compounds of the formula IIp, wherein Het_{A} corresponds to Het-R.183 as defined in table H, and Het_{B} corresponds for each individual compound to one row of table H.
Table 416. Compounds of the formula IIp, wherein Het_{A} corresponds to Het-R.184 as defined in table H, and Het_{B} corresponds for each individual compound to one row of table H.
Table 417. Compounds of the formula IIp, wherein Het_{A} corresponds to Het-R.185 as defined in table H, and Het_{B} corresponds for each individual compound to one row of table H.
Table 418. Compounds of the formula IIp, wherein Het_{A} corresponds to Het-R.186 as defined in table H, and Het_{B} corresponds for each individual compound to one row of table H.
Table 419. Compounds of the formula IIp, wherein Het_{A} corresponds to Het-R.187 as defined in table H, and Het_{B} corresponds for each individual compound to one row of table H.
Table 420. Compounds of the formula IIp, wherein Het_{A} corresponds to Het-R.188 as defined in table H, and Het_{B} corresponds for each individual compound to one row of table H.
Table 421. Compounds of the formula IIp, wherein Het_{A} corresponds to Het-R.189 as defined in table H, and Het_{B} corresponds for each individual compound to one row of table H.
Table 422. Compounds of the formula IIp, wherein Het_{A} corresponds to Het-R.190 as defined in table H, and Het_{B} corresponds for each individual compound to one row of table H.
Table 423. Compounds of the formula IIp, wherein Het_{A} corresponds to Het-R.191 as defined in table H, and Het_{B} corresponds for each individual compound to one row of table H.
Table 424. Compounds of the formula IIp, wherein Het_{A} corresponds to Het-R.192 as defined in table H, and Het_{B} corresponds for each individual compound to one row of table H.
Table 425. Compounds of the formula IIp, wherein Het_{A} corresponds to Het-R.193 as defined in table H, and Het_{B} corresponds for each individual compound to one row of table H.
Table 426. Compounds of the formula IIp, wherein Het_{A} corresponds to Het-R.194 as defined in table H, and Het_{B} corresponds for each individual compound to one row of table H.
Table 427. Compounds of the formula IIp, wherein Het_{A} corresponds to Het-R.195 as defined in table H, and Het_{B} corresponds for each individual compound to one row of table H.
Table 428. Compounds of the formula IIp, wherein Het_{A} corresponds to Het-R.196 as defined in table H, and Het_{B} corresponds for each individual compound to one row of table H.
Table 429. Compounds of the formula IIp, wherein Het_{A} corresponds to Het-R.197 as defined in table H, and Het_{B} corresponds for each individual compound to one row of table H.
Table 430. Compounds of the formula IIp, wherein Het_{A} corresponds to Het-R.198 as defined in table H, and Het_{B} corresponds for each individual compound to one row of table H.
Table 431. Compounds of the formula IIp, wherein Het_{A} corresponds to Het-R.199 as defined in table H, and Het_{B} corresponds for each individual compound to one row of table H.
Table 432. Compounds of the formula IIp, wherein Het_{A} corresponds to Het-R.200 as defined in table H, and Het_{B} corresponds for each individual compound to one row of table H.
Table 433. Compounds of the formula IIp, wherein Het_{A} corresponds to Het-R.201 as defined in table H, and Het_{B} corresponds for each individual compound to one row of table H.
Table 434. Compounds of the formula IIp, wherein Het_{A} corresponds to Het-R.202 as defined in table H, and Het_{B} corresponds for each individual compound to one row of table H.
Table 435. Compounds of the formula IIp, wherein Het_{A} corresponds to Het-R.203 as defined in table H, and Het_{B} corresponds for each individual compound to one row of table H.
Table 436. Compounds of the formula IIp, wherein Het_{A} corresponds to Het-R.204 as defined in table H, and Het_{B} corresponds for each individual compound to one row of table H.
Table 437. Compounds of the formula IIp, wherein Het_{A} corresponds to Het-R.205 as defined in table H, and Het_{B} corresponds for each individual compound to one row of table H.
Table 438. Compounds of the formula IIp, wherein Het_{A} corresponds to Het-R.206 as defined in table H, and Het_{B} corresponds for each individual compound to one row of table H.
Table 439. Compounds of the formula IIp, wherein Het_{A} corresponds to Het-R.207 as defined in table H, and Het_{B} corresponds for each individual compound to one row of table H.
Table 440. Compounds of the formula IIp, wherein Het_{A} corresponds to Het-R.208 as defined in table H, and Het_{B} corresponds for each individual compound to one row of table H.
Table 441. Compounds of the formula IIp, wherein Het_{A} corresponds to Het-R.209 as defined in table H, and Het_{B} corresponds for each individual compound to one row of table H.
Table 442. Compounds of the formula IIp, wherein Het_{A} corresponds to Het-R.210 as defined in table H, and Het_{B} corresponds for each individual compound to one row of table H.
Table 443. Compounds of the formula IIp, wherein Het_{A} corresponds to Het-R.211 as defined in table H, and Het_{B} corresponds for each individual compound to one row of table H.
Table 444. Compounds of the formula IIp, wherein Het_{A} corresponds to Het-R.212 as defined in table H, and Het_{B} corresponds for each individual compound to one row of table H.
Table 445. Compounds of the formula IIp, wherein Het_{A} corresponds to Het-R.213 as defined in table H, and Het_{B} corresponds for each individual compound to one row of table H.
Table 446. Compounds of the formula IIp, wherein Het_{A} corresponds to Het-R.214 as defined in table H, and Het_{B} corresponds for each individual compound to one row of table H.
Table 447. Compounds of the formula IIp, wherein Het_{A} corresponds to Het-R.215 as defined in table H, and Het_{B} corresponds for each individual compound to one row of table H.
Table 448. Compounds of the formula IIp, wherein Het_{A} corresponds to Het-R.216 as defined in table H, and Het_{B} corresponds for each individual compound to one row of table H.
Table 449. Compounds of the formula IIp, wherein Het_{A} corresponds to Het-R.217 as defined in table H, and Het_{B} corresponds for each individual compound to one row of table H.
Table 450. Compounds of the formula IIp, wherein Het_{A} corresponds to Het-R.218 as defined in table H, and Het_{B} corresponds for each individual compound to one row of table H.
Table 451. Compounds of the formula IIp, wherein Het_{A} corresponds to Het-R.219 as defined in table H, and Het_{B} corresponds for each individual compound to one row of table H.
Table 452. Compounds of the formula IIp, wherein Het_{A} corresponds to Het-R.220 as defined in table H, and Het_{B} corresponds for each individual compound to one row of table H.
Table 453. Compounds of the formula IIp, wherein Het_{A} corresponds to Het-R.221 as defined in table H, and Het_{B} corresponds for each individual compound to one row of table H.
Table 454. Compounds of the formula IIp, wherein Het_{A} corresponds to Het-R.222 as defined in table H, and Het_{B} corresponds for each individual compound to one row of table H.
Table 455. Compounds of the formula IIp, wherein Het_{A} corresponds to Het-R.223 as defined in table H, and Het_{B} corresponds for each individual compound to one row of table H.
Table 456. Compounds of the formula IIp, wherein Het_{A} corresponds to Het-R.224 as defined in table H, and Het_{B} corresponds for each individual compound to one row of table H.
Table 457. Compounds of the formula IIp, wherein Het_{A} corresponds to Het-R.225 as defined in table H, and Het_{B} corresponds for each individual compound to one row of table H.
Table 458. Compounds of the formula IIp, wherein Het_{A} corresponds to Het-R.226 as defined in table H, and Het_{B} corresponds for each individual compound to one row of table H.
Table 459. Compounds of the formula IIp, wherein Het_{A} corresponds to Het-R.227 as defined in table H, and Het_{B} corresponds for each individual compound to one row of table H.
Table 460. Compounds of the formula IIp, wherein Het_{A} corresponds to Het-R.228 as defined in table H, and Het_{B} corresponds for each individual compound to one row of table H.
Table 461. Compounds of the formula IIp, wherein Het_{A} corresponds to Het-R.229 as defined in table H, and Het_{B} corresponds for each individual compound to one row of table H.
Table 462. Compounds of the formula IIp, wherein Het_{A} corresponds to Het-R.230 as defined in table H, and Het_{B} corresponds for each individual compound to one row of table H.
Table 463. Compounds of the formula IIp, wherein Het_{A} corresponds to Het-R.231 as defined in table H, and Het_{B} corresponds for each individual compound to one row of table H.
Table 464. Compounds of the formula IIp, wherein Het_{A} corresponds to Het-R.232 as defined in table H, and Het_{B} corresponds for each individual compound to one row of table H.

Examples of preferred compounds II are also the compounds of formula II, wherein R¹, R², R³ are hydrogen, R^{4a}, R^{4b}, R^{4c} and R^{4d} are hydrogen, and R^{z} is acetyl, and wherein Het_{A} and Het_{B} are as defined in the tables 233 to 464.

### Preparation Methods

The compounds of formula I can be obtained as outlined in schemes 1 to 4.

The compounds of the formula I-A wherein X is oxygen or sulfur can be e.g. prepared from the corresponding aminothiocarbonyl-ethane compounds II-S and aminocarbonyl-ethane compounds II-O, respectively, as shown in scheme 1:

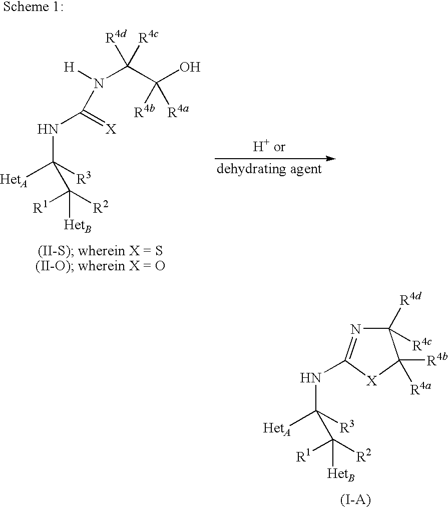

In scheme 1, R¹, R², R³, R^{4a}, R^{4b}, R^{4c}, R^{4d}, Het_{A} and Het_{B} are as defined above.

The aminothiocarbonylaminoethane compound II-S and the aminocarbonylamino-ethane compound II-O, respectively, can be cyclized by conventional means thereby obtaining the azoline compound of the formula I-A. Cyclization of the compound II-S and II-O, respectively, can be achieved e.g. under acid catalysis or under dehydrating conditions e.g. by Mitsunobu's reaction (see Tetrahedron Letters 1999, 40, 3125-3128) or as described below (preparation examples).

Alternatively, the compounds of the formula I-A according to the invention wherein X is O or S can be prepared by the method shown in scheme 2.

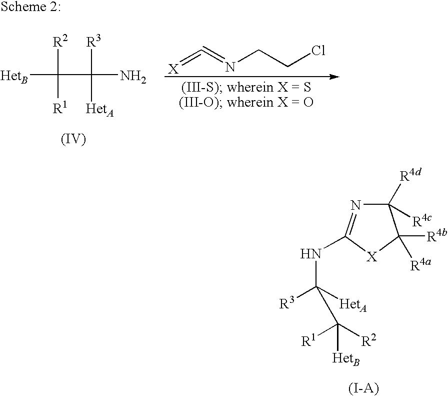

In scheme 2, R¹, R², R³, R^{4a}, R^{4b}, R^{4c}, R^{4d}, Het_{A} and Het_{B} are as defined above.

An amine IV or a salt thereof can be converted to an azoline I-A by reaction with 2-chloroethylisothiocyanate III-S or 2-chloroethylisocyanate III-O e.g. as described in Bio-org. Med. Chem. Lett. 1994, 4, 2317-22 and subsequent cyclization in the presence or absence of base.

1-Chloro-2-isothiocyanatoethane III-S (CAS-reg.-no.: 6099-88-3) and 2-chloroethylisocyanate III-O (CAS-reg.-no.: 1943-83-5) are commercially available.

Compounds of the formula I-AN wherein X is NR⁷ may be prepared by the method shown in scheme 3.

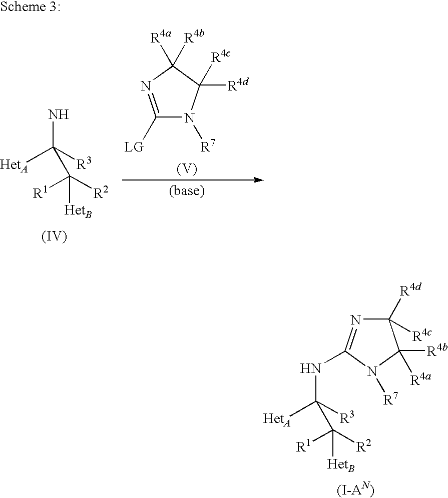

In scheme 3, R¹, R², R³, R^{4a}, R^{4b}, R^{4c}, R^{4d}, R⁷, Het_{A} and Het_{B} are as defined above and LG is a leaving group.

Compounds of the formula I-AN may be obtained by reacting an appropriate substituted amine IV or a salt thereof with a 2-substituted imidazoline V in an appropriate solvent. This reaction can be carried out, for example analogous to the methods described in U.S. Pat. No. 5,130,441 or EP 0389765.

Compounds of the formulae I-A¹ and I-A² wherein R⁵ and R⁶, respectively, are not hydrogen, can be obtained as outlined in scheme 4.

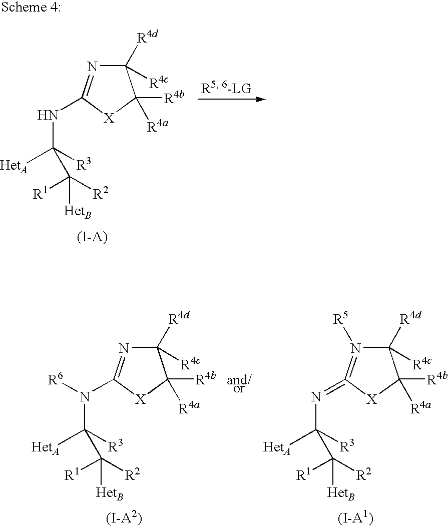

In scheme 4, R¹, R², R³, R^{4a}, R^{4b}, R^{4c}, R^{4d}, X, Het_{A} and Het_{B} are as defined above. A compound of formula I-A wherein R⁵ and R⁶, respectively, are hydrogen is treated with a suitable electrophile. Suitable electrophiles are e.g. an alkylating or acylating agent R^{5,6}-LG (LG=leaving group; e.g. Cl, Br, I, OSO₂R, OCO₂R, wherein the R is C₁-C₄-alkyl), e.g. as described in WO 2005063724.

Amines IV are known in the art or can be prepared by methods familiar to an organic chemist, for instance by application of general methods for the synthesis of amines described in J. Org. Chem. 1983, 48, 289-294. or Tetrahedron 1999, 55, 8883-8904 and as demonstrated below in the preparation procedure. Suitable amine salts IV are e.g. the acid addition salts formed by treating an amine IV with an inorganic or organic acid. Anions of useful acids are e.g. sulfate, hydrogensulfate, phosphate, dihydrogenphosphate, hydrogenphosphate, nitrate, bicarbonate, carbonate, chloride, bromide, p-toluene sulfonate, and the anions of C₁-C₄-alkanoic acids such as acetate, propionate, and the like.

Compounds of the formula II-S and II-O, respectively, can be prepared as shown in schemes 5 and 6 below.

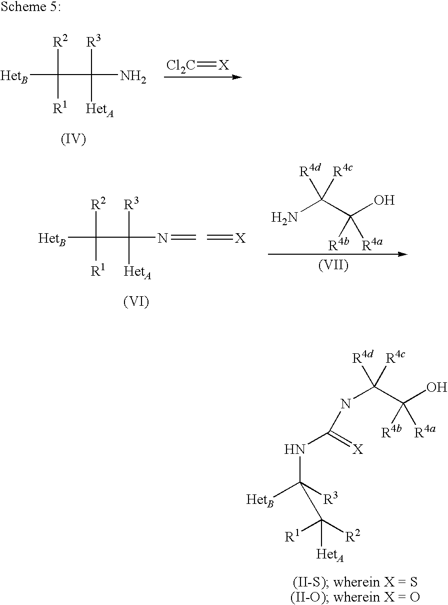

In scheme 5, R¹, R², R³, R^{4a}, R^{4b}, R^{4c}, R^{4d}, Het_{A} and Het_{B} are as defined above.

An amine IV or a salt thereof is converted to the corresponding iso(thio)cyanate VI by conventional means, e.g. by reacting IV with (thio)phosgene, as described for example in the case of thiophosgene in Houben-Weyl, E4, “Methoden der Organischen Chemie”, chapter IIc, pp. 837-842, Georg Thieme Verlag 1983. It may be advantageous to carry out the reaction in the presence of a base. The iso(thio)cyanate VI is then reacted with an aminoethanol VII to form an amino(thio)carbonylaminoethane compound. The reaction of the aminoethanol VII with iso(thio)cyanate V can be performed in accordance with standard methods of organic chemistry, see e.g. Biosci. Biotech. Biochem. 56 (7), 1062-65 (1992).

A further route to compounds II-S, wherein X is S is shown in scheme 6.

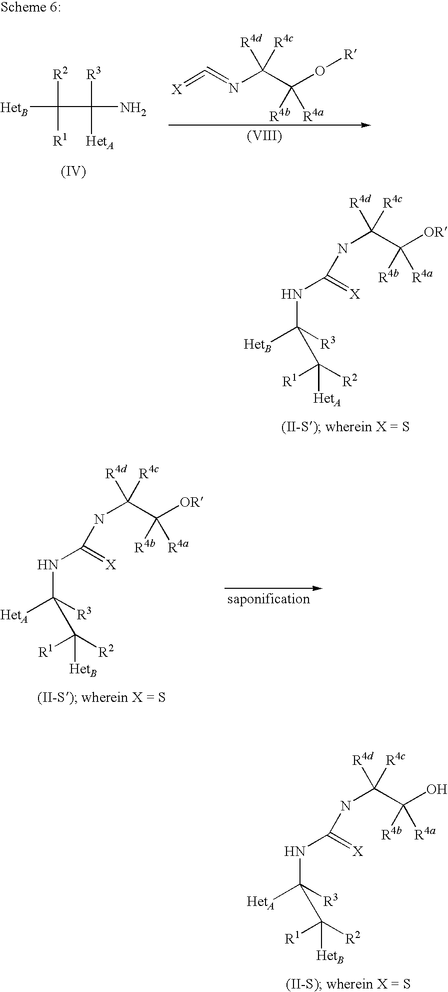

In scheme 6, R¹, R², R³, R^{4a}, R^{4b}, R^{4c}, R^{4d}, Het_{A} and Het_{B} are as defined above and R′ has the meanings given for R^{z} or is e.g. benzoyl.

An amine IV or a salt thereof can be converted to the corresponding aminothiocarbonylaminoethane compound II-S, by reacting the amine IV with an isothiocyanates VIII and subsequent saponification as described in the preparation examples below. Isothiocyanates VII can be prepared according to the procedures described in Coll. Czech. Chem. Commun. 1986, 51, 112-117.

Compounds of the formulae I, II, II-S and II-O, respectively, wherein Het_{A} and/or Het_{B} are a 5- or 6-membered nitrogen containing heteroaromatic ring may be converted to the corresponding N-oxides by treatment with a peracid under conditions known per se, for example by treating with hydrogen peroxide in an organic acid, such as formic acid, acetic acid, chloroacetic acid or trifluoroacetic acid (see, for example, J. Org. Chem. 55 (1990), 738-741 and Organic Synthesis, Collect. Vol. IV (1963), 655-656), or by reacting with an organic peracid, such as meta-perchlorobenzoic acid, in an inert solvent, for example a halogenated hydrocarbon, such as dichloromethane or dichloroethane (see, for example, Synthetic Commun. 22 (18) (1992), 2645; J. Med. Chem. (1998), 2146).

### Pests

Due to their excellent activity, the compounds of the formula I and formula II may be used for controlling animal pests, in particular selected harmful insects, arachnids and nematodes.

The compounds of the formula I and formula II are especially suitable for efficiently combating the following pests:

Insects from the order of the lepidopterans (Lepidoptera), for example Agrotis ypsilon, Agrotis segetum, Alabama argillacea, Anticarsia gemmatalis, Argyresthia conjugella, Autographa gamma, Bupalus piniarius, Cacoecia murinana, Capua reticulana, Chematobia brumata, Choristoneura fumiferana, Choristoneura occidentalis, Cirphis unipuncta, Cydia pomonella, Dendrolimus pini, Diaphania nitidalis, Diatraea grandiosella, Earias insulana, Elasmopalpus lignosellus, Eupoecilia ambiguella, Evetria bouliana, Feltia subterranea, Galleria mellonella, Grapholitha funebrana, Grapholitha molesta, Heliothis armigera, Heliothis virescens, Heliothis zea, Hellula undalis, Hibernia defoliaria, Hyphantria cunea, Hyponomeuta malinellus, Keiferia lycopersicella, Lambdina fiscellaria, Laphygma exigua, Leucoptera coffeella, Leucoptera scitella, Lithocolletis blancardella, Lobesia botrana, Loxostege sticticalis, Lymantria dispar, Lymantria monacha, Lyonetia clerkella, Malacosoma neustria, Mamestra brassicae, Orgyia pseudotsugata, Ostrinia nubilalis, Panolis flammea, Pectinophora gossypiella, Peridroma saucia, Phalera bucephala, Phthorimaea operculella, Phyllocnistis citrella, Pieris brassicae, Plathypena scabra, Plutella xylostella, Pseudoplusia includens, Rhyacionia frustrana, Scrobipalpula absoluta, Sitotroga cerealella, Sparganothis pilleriana, Spodoptera frugiperda, Spodoptera littoralis, Spodoptera litura, Thaumatopoea pityocampa, Tortrix viridana, Trichoplusia ni and Zeiraphera canadensis,

beetles (Coleoptera), for example Agrilus sinuatus, Agriotes lineatus, Agriotes obscurus, Amphimallus solstitialis, Anisandrus dispar, Anthonomus grandis, Anthonomus pomorum, Aphthona euphoridae, Athous haemorrhoidalis, Atomaria linearis, Blastophagus piniperda, Blitophaga undata, Bruchus rufimanus, Bruchus pisorum, Bruchus lentis, Byctiscus betulae, Cassida nebulosa, Cerotoma trifurcata, Cetonia aurata, Ceuthorrhynchus assimilis, Ceuthorrhynchus napi, Chaetocnema tibialis, Conoderus vespertinus, Crioceris asparagi, Ctenicera ssp., Diabrotica longicornis, Diabrotica semipunctata, Diabrotica 12-punctata Diabrotica speciosa, Diabrotica virgifera, Epilachna varivestis, Epitrix hirtipennis, Eutinobothrus brasiliensis, Hylobius abietis, Hypera brunneipennis, Hypera postica, Ips typographus, Lema bilineata, Lema melanopus, Leptinotarsa decemlineata, Limonius californicus, Lissorhoptrus oryzophilus, Melanotus communis, Meligethes aeneus, Melolontha hippocastani, Melolontha melolontha, Oulema oryzae, Otiorrhynchus sulcatus, Otiorrhynchus ovatus, Phaedon cochleariae, Phyllobius pyri, Phyllotreta chrysocephala, Phyllophaga sp., Phyllopertha horticola, Phyllotreta nemorum, Phyllotreta striolata, Popillia japonica, Sitona lineatus and Sitophilus granaria,

flies, mosquitoes (Diptera), e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Ceratitis capitata, Chrysomya bezziana, Chrysomya hominivorax, Chrysomya macellaria, Chrysops discalis, Chrysops silacea, Chrysops atlanticus, Cochliomyia hominivorax, Contarinia sorghicola Cordylobia anthropophaga, Culicoides furens, Culex pipiens, Culex nigripalpus, Culex quinquefasciatus, Culex tarsalis, Culiseta inornata, Culiseta melanura, Dacus cucurbitae, Dacus oleae, Dasineura brassicae, Delia antique, Delia coarctata, Delia platura, Delia radicum, Dermatobia hominis, Fannia canicularis, Geomyza Tripunctata, Gasterophilus intestinalis, Glossina morsitans, Glossina palpalis, Glossina fuscipes, Glossina tachinoides, Haematobia irritans, Haplodiplosis equestris, Hippelates spp., Hylemyia platura, Hypoderma lineata, Leptoconops torrens, Liriomyza sativae, Liriomyza trifolii, Lucilia caprina, Lucilia cuprina, Lucilia sericata, Lycoria pectoralis, Mansonia titillanus, Mayetiola destructor, Musca autumnalis, Musca domestica, Muscina stabulans, Oestrus ovis, Opomyza florum, Oscinella frit, Pegomya hysocyami, Phorbia antiqua, Phorbia brassicae, Phorbia coarctata, Phlebotomus argentipes, Psorophora columbiae, Psila rosae, Psorophora discolor, Prosimulium mixtum, Rhagoletis cerasi, Rhagoletis pomonella, Sarcophaga haemorrhoidalis, Sarcophaga spp., Simulium vittatum, Stomoxys calcitrans, Tabanus bovinus, Tabanus atratus, Tabanus lineola, and Tabanus similis, Tipula oleracea, and Tipula paludosa

thrips (Thysanoptera), e.g. Dichromothrips corbetti, Dichromothrips ssp., Frankliniella fusca, Frankliniella occidentalis, Frankliniella tritici, Scirtothrips citri, Thrips oryzae, Thrips palmi and Thrips tabaci,

termites (Isoptera), e.g. Calotermes flavicollis, Leucotermes flavipes, Heterotermes aureus, Reticulitermes flavipes, Reticulitermes virginicus, Reticulitermes lucifugus, Reticulitermes santonensis, Reticulitermes grassei, Termes natalensis, and Coptotermes formosanus,

cockroaches (Blattaria-Blattodea), e.g. Blattella germanica, Blattella asahinae, Periplaneta americana, Periplaneta japonica, Periplaneta brunnea, Periplaneta fuligginosa, Periplaneta australasiae, and Blatta orientalis,

bugs, aphids, leafhoppers, whiteflies, scale insects, cicadas (Hemiptera), e.g. Acrosternum hilare, Blissus leucopterus, Cyrtopeltis notatus, Dysdercus cingulatus, Dysdercus intermedius, Eurygaster integriceps, Euschistus impictiventris, Leptoglossus phyllopus, Lygus lineolaris, Lygus pratensis, Nezara viridula, Piesma quadrata, Solubea insularis, Thyanta perditor, Acyrthosiphon onobrychis, Adelges laricis, Aphidula nasturtii, Aphis fabae, Aphis forbesi, Aphis pomi, Aphis gossypii, Aphis grossulariae, Aphis schneideri, Aphis spiraecola, Aphis sambuci, Acyrthosiphon pisum, Aulacorthum solani, Bemisia argentifolii, Brachycaudus cardui, Brachycaudus helichrysi, Brachycaudus persicae, Brachycaudus prunicola, Brevicoryne brassicae, Capitophorus horni, Cerosipha gossypii, Chaetosiphon fragaefolii, Cryptomyzus ribis, Dreyfusia nordmannianae, Dreyfusia piceae, Dysaphis radicola, Dysaulacorthum pseudosolani, Dysaphis plantaginea, Dysaphis pyri, Empoasca fabae, Hyalopterus pruni, Hyperomyzus lactucae, Macrosiphum avenae, Macrosiphum euphorbiae, crosiphum avenae, Macrosiphum euphorbiae, Macrosiphon rosae, Megoura viciae, Melanaphis pyrarius, Metopolophium dirhodum, Myzus persicae, Myzus ascalonicus, Myzus cerasi, Myzus varians, Nasonovia ribis-nigri, Nilaparvata lugens, Pemphigus bursarius, Perkinsiella saccharicida, Phorodon humuli, Psylla mali, Psylla piri, Rhopalomyzus ascalonicus, Rhopalosiphum maidis, Rhopalosiphum padi, Rhopalosiphum insertum, Sappaphis mala, Sappaphis mali, Schizaphis graminum, Schizoneura lanuginosa, Sitobion avenae, Trialeurodes vaporariorum, Toxoptera aurantiiand, Viteus vitifolii, Cimex lectularius, Cimex hemipterus, Reduvius senilis, Triatoma spp., and Arilus critatus.

ants, bees, wasps, sawflies (Hymenoptera), e.g. Athalia rosae, Atta cephalotes, Atta capiguara, Atta cephalotes, Atta laevigata, Atta robusta, Atta sexdens, Atta texana, Crematogaster spp., Hoplocampa minuta, Hoplocampa testudinea, Lasius niger, Monomorium pharaonis, Solenopsis geminata, Solenopsis invicta, Solenopsis richteri, Solenopsis xyloni, Pogonomyrmex barbatus, Pogonomyrmex californicus, Pheidole megacephala, Dasymutilla occidentalis, Bombus spp., Vespula squamosa, Paravespula vulgaris, Paravespula pennsylvanica, Paravespula germanica, Dolichovespula maculata, Vespa crabro, Polistes rubiginosa, Camponotus floridanus, and Linepithema humile,

crickets, grasshoppers, locusts (Orthoptera), e.g. Acheta domestica, Gryllotalpa gryllotalpa, Locusta migratoria, Melanoplus bivittatus, Melanoplus femurrubrum, Melanoplus mexicanus, Melanoplus sanguinipes, Melanoplus spretus, Nomadacris septemfasciata, Schistocerca americana, Schistocerca gregaria, Dociostaurus maroccanus, Tachycines asynamorus, Oedaleus senegalensis, Zonozerus variegatus, Hieroglyphus daganensis, Kraussaria angulifera, Calliptamus italicus, Chortoicetes terminifera, and Locustana pardalina,

Arachnoidea, such as arachnids (Acarina), e.g. of the families Argasidae, Ixodidae and Sarcoptidae, such as Amblyomma americanum, Amblyomma variegatum, Ambryomma maculatum, Argas persicus, Boophilus annulatus, Boophilus decoloratus, Boophilus microplus, Dermacentor silvarum, Dermacentor andersoni, Dermacentor variabilis, Hyalomma truncatum, Ixodes ricinus, Ixodes rubicundus, Ixodes scapularis, Ixodes holocyclus, Ixodes pacificus, Ornithodorus moubata, Ornithodorus hermsi, Ornithodorus turicata, Ornithonyssus bacoti, Otobius megnini, Dermanyssus gallinae, Psoroptes ovis, Rhipicephalus sanguineus, Rhipicephalus appendiculatus, Rhipicephalus evertsi, Sarcoptes scabiei, and Eriophyidae spp. such as Aculus schlechtendali, Phyllocoptrata oleivora and Eriophyes sheldoni; Tarsonemidae spp. such as Phytonemus pallidus and Polyphagotarsonemus latus; Tenuipalpidae spp. such as Brevipalpus phoenicis; Tetranychidae spp. such as Tetranychus cinnabarinus, Tetranychus kanzawai, Tetranychus pacificus, Tetranychus telarius and Tetranychus urticae, Panonychus ulmi, Panonychus citri, and Oligonychus pratensis; Araneida, e.g. chus citri, and Oligonychus pratensis; Araneida, e.g. Latrodectus mactans, and Loxosceles reclusa,

fleas (Siphonaptera), e.g. Ctenocephalides felis, Ctenocephalides canis, Xenopsylla cheopis, Pulex irritans, Tunga penetrans, and Nosopsyllus fasciatus,

silverfish, firebrat (Thysanura), e.g. Lepisma saccharina and Thermobia domestica,

centipedes (Chilopoda), e.g. Scutigera coleoptrata,

millipedes (Diplopoda), e.g. Narceus spp.,

Earwigs (Dermaptera), e.g. forficula auricularia,

lice (Phthiraptera), e.g. Pediculus humanus capitis, Pediculus humanus corporis, Pthirus pubis, Haematopinus eurysternus, Haematopinus suis, Linognathus vituli, Bovicola bovis, Menopon gallinae, Menacanthus stramineus and Solenopotes capillatus.

In a preferred embodiment of the invention the compounds of formula I and of formula II are used for controlling insects or arachnids, in particular insects of the orders Lepidoptera, Coleoptera and Homoptera and arachnids of the order Acarina. The compounds of the formula I and of formula II according to the present invention are particularly useful for controlling insects of the order Lepidoptera and Homoptera.

### Formulations

For use in a method according to the present invention, the compounds of formula I can be converted into the customary formulations, e.g. solutions, emulsions, suspensions, dusts, powders, pastes, granules and directly sprayable solutions. The use form depends on the particular purpose and application method. Formulations and application methods are chosen to ensure in each case a fine and uniform distribution of the compound of the formula I according to the present invention.

Accordingly, the invention further provides e.g. an agriculturally composition for combating such animal pests, which comprises such an amount of at least one compound of the formula I or at least an agriculturally useful salt of I and at least one inert liquid and/or solid agronomically acceptable carrier that it has a pesticidal action and, if desired, at least one surfactant.

Such a composition may contain a single active compound of the formula I, or the enantiomers thereof or a mixture of several active compounds of formula I according to the present invention. The composition according to the present invention may comprise an individual isomer or mixtures of isomers as well as individual tautomers or mixtures of tautomers.

The formulations are prepared in a known manner (see e.g. for review U.S. Pat. No. 3,060,084, EP-A 707 445 (for liquid concentrates), Browning, “Agglomeration”, Chemical Engineering, Dec. 4, 1967, 147-48, Perry's Chemical Engineer's Handbook, 4th Ed., McGraw-Hill, New York, 1963, pages 8-57 and et seq. WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2,095,558, U.S. Pat. No. 3,299,566, Klingman, Weed Control as a Science, John Wiley and Sons, Inc., New York, 1961, Hance et al., Weed Control Handbook, 8th Ed., Blackwell Scientific Publications, Oxford, 1989 and Mollet, H., Grubemann, A., Formulation technology, Wiley VCH Verlag GmbH, Weinheim (Germany), 2001, 2. D. A. Knowles, Chemistry and Technology of Agrochemical Formulations, Kluwer Academic Publishers, Dordrecht, 1998 (ISBN 0-7514-0443-8), for example by extending the active compound with auxiliaries suitable for the formulation of agrochemicals, such as solvents and/or carriers, if desired emulsifiers, surfactants and dispersants, preservatives, anti-foaming agents, anti-freezing agents, for seed treatment formulation also optionally colorants and/or binders and/or gelling agents.

Solvents/carriers, which are suitable, are e.g.:
solvents such as water, aromatic solvents (for example Solvesso products, xylene and the like), paraffins (for example mineral fractions), alcohols (for example methanol, butanol, pentanol, benzyl alcohol), ketones (for example cyclohexanone, gamma-butyrolactone), pyrrolidones (N-methyl-pyrrolidone (NMP), N-octylpyrrolidone NOP), acetates (glycol diacetate), alkyl lactates, lactones such as g-butyrolactone, glycols, fatty acid dimethylamides, fatty acids and fatty acid esters, triglycerides, oils of vegetable or animal origin and modified oils such as alkylated plant oils. In principle, solvent mixtures may also be used.
carriers such as ground natural minerals and ground synthetic minerals, such as silica gels, finely divided silicic acid, silicates, talc, kaolin, attaclay, limestone, lime, chalk, bole, loess, clay, dolomite, diatomaceous earth, calcium sulfate and magnesium sulfate, magnesium oxide, ground synthetic materials, fertilizers, such as, for example, ammonium sulfate, ammonium phosphate, ammonium nitrate, ureas and products of vegetable origin, such as cereal meal, tree bark meal, wood meal and nutshell meal, cellulose powders and other solid carriers.

Suitable emulsifiers are nonionic and anionic emulsifiers (for example polyoxyethylene fatty alcohol ethers, alkylsulfonates and arylsulfonates).

Examples of dispersants are lignin-sulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters,

Also anti-freezing agents such as glycerin, ethylene glycol, propylene glycol and bactericides such as can be added to the formulation.

Suitable antifoaming agents are for example antifoaming agents based on silicon or magnesium stearate.

Suitable preservatives are for example dichlorophen und benzyl alcohol hemiformal

Suitable thickeners are compounds, which confer a pseudoplastic flow behavior to the formulation, i.e. high viscosity at rest and low viscosity in the agitated stage. Mention may be made, in this context, for example, of commercial thickeners based on poly-saccharides, such as Xanthan Gum® (Kelzan® from Kelco), Rhodopol®23 (Rhone Poulenc) or Veegum® (from R.T. Vanderbilt), or organic phyllosilicates, such as Attaclay® (from Engelhardt). Antifoam agents suitable for the dispersions according to the invention are, for example, silicone emulsions (such as, for example, Silikon® SRE, Wacker or Rhodorsil® from Rhodia), long-chain alcohols, fatty acids, organofluorine compounds and mixtures thereof. Biocides can be added to stabilize the compositions according to the invention against attack by microorganisms. Suitable biocides are, for example, based on isothiazolones such as the compounds marketed under the trademarks Proxel® from Avecia (or Arch) or Acticide® RS from Thor Chemie and Kathon® MK from Rohm & Haas. Suitable antifreeze agents are organic polyols, for example ethylene glycol, propylene glycol or glycerol. These are usually employed in amounts of not more than 10% by weight, based on the total weight of the active compound composition. If appropriate, the active compound compositions according to the invention may comprise 1 to 5% by weight of buffer, based on the total amount of the formulation prepared, to regulate the pH, the amount and type of the buffer used depending on the chemical properties of the active compound or the active compounds. Examples of buffers are alkali metal salts of weak inorganic or organic acids, such as, for example, phosphoric acid, boronic acid, acetic acid, propionic acid, citric acid, fumaric acid, tartaric acid, oxalic acid and succinic acid.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dusts can be prepared by mixing or concomitantly grinding the active substances with a solid carrier.

Granules, for example coated granules, impregnated granules and homogeneous granules, can be prepared by binding the active ingredients to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active ingredient. The active ingredients are employed in a purity of from 90% to 100%, preferably 95% to 100% (according to NMR spectrum).

For seed treatment purposes, respective formulations can be diluted 2-10 fold leading to concentrations in the ready to use preparations of 0.01 to 60% by weight active compound by weight, preferably 0.1 to 40% by weight.

The compound of formula I can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; they are intended to ensure in each case the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active ingredient concentrations in the ready-to-use products can be varied within relatively wide ranges. In general, they are from 0.0001 to 10%, preferably from 0.01 to 1% per weight.

The active ingredients may also be used successfully in the ultra-low-volume process (ULV), it being possible to apply formulations comprising over 95% by weight of active ingredient, or even to apply the active ingredient without additives.

The following are examples of formulations:

1. Products for dilution with water. For seed treatment purposes, such products may be applied to the seed diluted or undiluted.

### A) Water-Soluble Concentrates (SL, LS)

10 parts by weight of the active compound is dissolved in 90 parts by weight of water or a water-soluble solvent. As an alternative, wetters or other auxiliaries are added. The active compound dissolves upon dilution with water, whereby a formulation with 10% (w/w) of active compound is obtained.

### B) Dispersible Concentrates (DC)

20 parts by weight of the active compound is dissolved in 70 parts by weight of cyclo-hexanone with addition of 10 parts by weight of a dispersant, for example polyvinylpyrrolidone. Dilution with water gives a dispersion, whereby a formulation with 20% (w/w) of active compounds is obtained.

### C) Emulsifiable Concentrates (EC)

15 parts by weight of the active compounds is dissolved in 7 parts by weight of xylene with addition of calcium dodecylbenzenesulfonate and castor oil ethoxylate (in each case 5 parts by weight). Dilution with water gives an emulsion, whereby a formulation with 15% (w/w) of active compounds is obtained.

### D) Emulsions (EW, EO, ES)

25 parts by weight of the active compound is dissolved in 35 parts by weight of xylene with addition of calcium dodecylbenzenesulfonate and castor oil ethoxylate (in each case 5 parts by weight). This mixture is introduced into 30 parts by weight of water by means of an emulsifier machine (e.g. Ultraturrax) and made into a homogeneous emulsion. Dilution with water gives an emulsion, whereby a formulation with 25% (w/w) of active compound is obtained.

### E) Suspensions (SC, OD, FS)

In an agitated ball mill, 20 parts by weight of the active compound is comminuted with addition of 10 parts by weight of dispersants, wetters and 70 parts by weight of water or of an organic solvent to give a fine active compound suspension. Dilution with water gives a stable suspension of the active compound, whereby a formulation with 20% (w/w) of active compound is obtained.

### F) Water-Dispersible Granules and Water-Soluble Granules (WG, SG)

50 parts by weight of the active compound is ground finely with addition of 50 parts by weight of dispersants and wetters and made as water-dispersible or water-soluble granules by means of technical appliances (for example extrusion, spray tower, fluidized bed). Dilution with water gives a stable dispersion or solution of the active compound, whereby a formulation with 50% (w/w) of active compound is obtained.

### G) Water-Dispersible Powders and Water-Soluble Powders (WP, SP, SS, WS)

75 parts by weight of the active compound are ground in a rotor-stator mill with addition of 25 parts by weight of dispersants, wetters and silica gel. Dilution with water gives a stable dispersion or solution of the active compound, whereby a formulation with 75% (w/w) of active compound is obtained.

### H) Gel-Formulation (GF)

In an agitated ball mill, 20 parts by weight of the active compound is comminuted with addition of 10 parts by weight of dispersants, 1 part by weight of a gelling agent wetters and 70 parts by weight of water or of an organic solvent to give a fine active compound suspension. Dilution with water gives a stable suspension of the active compound, whereby a formulation with 20% (w/w) of active compound is obtained.

2. Products to be applied undiluted for foliar applications. For seed treatment purposes, such products may be applied to the seed diluted or undiluted.

### I) Dustable Powders (DP, DS)

5 parts by weight of the active compound are ground finely and mixed intimately with 95 parts by weight of finely divided kaolin. This gives a dustable product having 5% (w/w) of active compound.

### J) Granules (GR, FG, GG, MG)

0.5 part by weight of the active compound is ground finely and associated with 95.5 parts by weight of carriers, whereby a formulation with 0.5% (w/w) of active compound is obtained. Current methods are extrusion, spray-drying or the fluidized bed. This gives granules to be applied undiluted for foliar use.

### K) ULV Solutions (UL)

10 parts by weight of the active compound is dissolved in 90 parts by weight of an organic solvent, for example xylene. This gives a product having 10% (w/w) of active compound, which is applied undiluted for foliar use.

Various types of oils, wetters, adjuvants, herbicides, fungicides, other pesticides, or bactericides may be added to the active ingredients, if appropriate just immediately prior to use (tank mix). These agents usually are admixed with the agents according to the invention in a weight ratio of 1:10 to 10:1.

The compounds and compositions of the present invention compounds I may be applied with other active ingredients, for example with other pesticides, insecticides, herbicides, fertilizers such as ammonium nitrate, urea, potash, and superphosphate, phytotoxicants and plant growth regulators, safeners and nematicides. These additional ingredients may be used sequentially or in combination with the above-described compositions, if appropriate also added only immediately prior to use (tank mix). For example, the plant(s) may be sprayed with a composition of this invention either before or after being treated with other active ingredients.

These additional agents can be admixed with the agents used according to the invention in a weight ratio of 1:10 to 10:1. Mixing the compounds I or the compositions comprising them in the use form as pesticides with other pesticides frequently results in a broader pesticidal spectrum of action.

The following list M of pesticides together with which the compounds according to the invention can be used and with which potential synergistic effects might be produced, is intended to illustrate the possible combinations, but not to impose any limitation:

M.1. Organo(thio)phosphates: acephate, azamethiphos, azinphos-ethyl, azinphos-methyl, chlorethoxyfos, chlorfenvinphos, chlormephos, chlorpyrifos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, flupyrazophos, fosthiazate, heptenophos, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, trichlorfon, vamidothion;
M.2. Carbamates: aldicarb, alanycarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, trimethacarb, XMC, xylylcarb, triazamate;
M.3. Pyrethroids: acrinathrin, allethrin, d-cis-trans allethrin, d-trans allethrin, bifenthrin, bioallethrin, bioallethrin S-cylclopentenyl, bioresmethrin, cycloprothrin, cyfluthrin, beta-, yfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin, deltamethrin, empenthrin, esfenvalerate, etofenprox, fenpropathrin, fenvalerate, flucythrinate, flumethrin, tau-fluvalinate, halfenprox, imiprothrin, permethrin, phenothrin, prallethrin, resmethrin, RU 15525, silafluofen, tefluthrin, tetramethrin, tralomethrin, transfluthrin, ZXI 8901;
M.4. Juvenile hormone mimics: hydroprene, kinoprene, methoprene, fenoxycarb, pyriproxyfen;
M.5. Nicotinic receptor agonists/antagonists compounds: acetamiprid, bensultap, cartap hydrochloride, clothianidin, dinotefuran, imidacloprid, thiamethoxam, nitenpyram, nicotine, spinosad (allosteric agonist), thiacloprid, thiocyclam, thiosultap-sodium and AKD1022.
M.6. GABA gated chloride channel antagonist compounds: chlordane, endosulfan, gamma-HCH (lindane); acetoprole, ethiprole, fipronil, pyrafluprole, pyriprole, vaniliprole, the phenylpyrazole compound of formula M^{6.1}

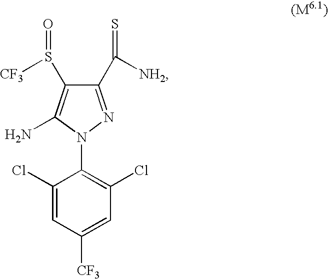

M.7. Chloride channel activators: abamectin, emamectin benzoate, milbemectin, lepimectin;
M.8. METI I compounds: fenazaquin, fenpyroximate, pyrimidifen, pyridaben, tebufenpyrad, tolfenpyrad, flufenerim, rotenone;
M.9. METI II and III compounds: acequinocyl, fluacyprim, hydramethylnon;
M.10. Uncouplers of oxidative phosphorylation: chlorfenapyr, DNOC;
M. 11. Inhibitors of oxidative phosphorylation: azocyclotin, cyhexatin, diafenthiuron, fenbutatin oxide, propargite, tetradifon;
M.12. Moulting disruptors: cyromazine, chromafenozide, halofenozide, methoxyfenozide, tebufenozide;
M.13. Synergists: piperonyl butoxide, tribufos;
M.14. Sodium channel blocker compounds: indoxacarb, metaflumizone;
M.15. Fumigants: methyl bromide, chloropicrin sulfuryl fluoride;
M.16. Selective feeding blockers: crylotie, pymetrozine, flonicamid;
M.17. Mite growth inhibitors: clofentezine, hexythiazox, etoxazole;
M.18. Chitin synthesis inhibitors: buprofezin, bistrifluoron, chlorfluazuron, diflubenzuron, flucycloxuron, flufenoxuron, hexaflumuron, lufenuron, novaluron, noviflumuron, teflubenzuron, triflumuron;
M.19. Lipid biosynthesis inhibitors: spirodiclofen, spiromesifen, spirotetramat;
M.20. octapaminergic agonsits: amitraz;
M.21. ryanodine receptor modulators: flubendiamide;
M.22. Various: aluminium phosphide, amidoflumet, benclothiaz, benzoximate, bifenazate, borax, bromopropylate, cyanide, cyenopyrafen, cyflumetofen, chinomethionate, dicofol, fluoroacetate, phosphine, pyridalyl, pyrifluquinazon, sulfur, organic sulfur compounds, tartar emetic; pyrimidinyl alkynylether compounds M^{22.1} or thiadiazolyl alkynylether compounds M^{22.2}:

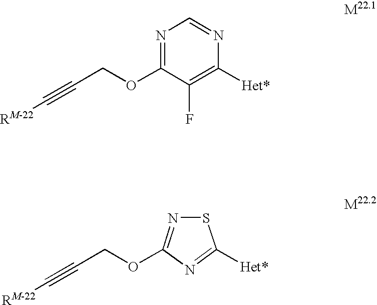

wherein R^{M-22} is methyl or ethyl and Het* is 3,3-dimethylpyrrolidin-1-yl, 3-methylpiperidin-1-yl, 3,5-dimethylpiperidin-1-yl, 3-trifluormethylpiperidin-1-yl, hexa-hydroazepin-1-yl, 2,6-dimethylhexahydroazepin-1-yl or 2,6-dimethylmorpholin-4-yl.
M.23. N—R′-2,2-dihalo-1-R″cyclo-propanecarboxamide-2-(2,6-dichloro-α,α,α-tri-fluoro-p-tolyl)hydrazone or N—R′-2,2-di(R′″)propionamide-2-(2,6-dichloro-α,α,α-trifluoro-p-tolyl)-hydrazone, wherein R′ is methyl or ethyl, halo is chloro or bromo, R″ is hydrogen or methyl and R′″ is methyl or ethyl;
M.24. Anthranilamides: chloranthraniliprole, the compound of formula M²⁴ 1

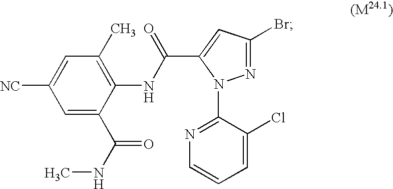

M.25. Malononitrile compounds: CF₂HCF₂CF₂CF₂CH₂C(CN)₂CH₂CH₂CF₃ (2-(2,2,3,3,4,4,5,5-octafluoropentyl)-2-(3,3,3-trifluoro-propyl)malononitrile), CF₃(CH₂)₂C(CN)₂CH₂(CF₂)₅CF₂H, (2-(2,2,3,3,4,4,5,5,6,6,7,7-Dodecafluoro-heptyl)-2-(3,3,3-trifluoro-propyl)-malononitrile), CF₃(CH₂)₂C(CN)₂(CH₂)₂C(CF₃)₂F (2-(3,4,4,4-Tetrafluoro-3-trifluoromethyl-butyl)-2-(3,3,3-trifluoro-propyl)-malononitrile), CF₃(CH₂)₂C(CN)₂(CH₂)₂(CF₂)₃CF₃ (2-(3,3,4,4,5,5,6,6,6-Nonafluoro-hexyl)-2-(3,3,3-trifluoro-propyl)-malononitrile), CF₂H(CF₂)₃CH₂C(CN)₂CH₂(CF₂)₃CF₂H (2,2-Bis-(2,2,3,3,4,4,5,5-octafluoro-pentyl)-malononitrile), CF₃(CH₂)₂C(CN)₂CH₂(CF₂)₃CF₃ (2-(2,2,3,3,4,4,5,5,5-Nonafluoro-pentyl)-2-(3,3,3-trifluoro-propyl)-malononitrile), CF₃(CF₂)₂CH₂C(CN)₂CH₂(CF₂)₃CF₂H (2-(2,2,3,3,4,4,4-Heptafluoro-butyl)-2-(2,2,3,3,4,4,5,5-octafluoro-pentyl)-malononitrile), CF₃CF₂CH₂C(CN)₂CH₂(CF₂)₃CF₂H (2-(2,2,3,3,4,4,5,5-Octafluoro-pentyl)-2-(2,2,3,3,3-pentafluoro-propyl)-malononitrile), CF₂HCF₂CF₂CF₂CH₂C(CN)₂CH₂CH₂CF₂CF₃ (2-(2,2,3,3,4,4,5,5-octafluoropentyl)-2-(3,3,4,4,4-pentafluorobutyl)-malonodinitrile), CF₃(CH₂)₂C(CN)₂CH₂(CF₂)₃CF₂H (2-(2,2,3,3,4,4,5,5-octafluoropentyl)-2-(3,3,3-trifluoro-butyl)-malononitrile);
M.26. Microbial disruptors: Bacillus thuringiensis subsp. Israelensi, Bacillus sphaericus, Bacillus thuringiensis subsp. Aizawai, Bacillus thuringiensis subsp. Kurstaki, Bacillus thuringiensis subsp. Tenebrionis;

The commercially available compounds of the group M may be found in The Pesticide Manual, 13^{th} Edition, British Crop Protection Council (2003) among other publications.

Thioamides of formula M^{6.1} and their preparation have been described in WO 98/28279. Lepimectin is known from Agro Project, PJB Publications Ltd, November 2004. Benclothiaz and its preparation have been described in EP-A1454621. Methidathion and Paraoxon and their preparation have been described in Farm Chemicals Handbook, Volume 88, Meister Publishing Company, 2001. Acetoprole and its preparation have been described in WO 98/28277. Metaflumizone and its preparation have been described in EP-A1462 456. Flupyrazofos has been described in Pesticide Science 54, 1988, p. 237-243 and in U.S. Pat. No. 4,822,779. Pyrafluprole and its preparation have been described in JP 2002193709 and in WO 01/00614. Pyriprole and its preparation have been described in WO 98/45274 and in U.S. Pat. No. 6,335,357. Amidoflumet and its preparation have been described in U.S. Pat. No. 6,221,890 and in JP 21010907. Flufenerim and its preparation have been described in WO 03/007717 and in WO 03/007718. AKD 1022 and its preparation have been described in U.S. Pat. No. 6,300,348. Chloranthraniliprole has been described in WO 01/70671, WO 03/015519 and WO 05/118552. Anthranilamide derivatives of formula M^{24.1} have been described in WO 01/70671, WO 04/067528 and WO 05/118552. Cyflumetofen and its preparation have been described in WO 04/080180. The aminoquinazolinone compound pyrifluquinazon has been described in EP A 109 7932. The alkynylether compounds M^{22.1} and M^{22.2} are described e.g. in JP 2006131529. Organic sulfur compounds have been described in WO 2007060839. The malononitrile compounds have been described in WO 02/089579, WO 02/090320, WO 02/090321, WO 04/006677, WO 05/068423, WO 05/068432 and WO 05/063694.

Fungicidal mixing partners are those selected from the group F consisting of

F.1 acylalanines such as benalaxyl, metalaxyl, ofurace, oxadixyl;
F.2 amine derivatives such as aldimorph, dodine, dodemorph, fenpropimorph, fenpropidin, guazatine, iminoctadine, spiroxamin, tridemorph;
F.3 anilinopyrimidines such as pyrimethanil, mepanipyrim or cyrodinyl;
F.4 antibiotics such as cycloheximid, griseofulvin, kasugamycin, natamycin, polyoxin or streptomycin;
F.5 azoles such as bitertanol, bromoconazole, cyproconazole, difenoconazole, dinitroconazole, epoxiconazole, fenbuconazole, fluquiconazole, flusilazole, hexaconazole, imazalil, metconazole, myclobutanil, penconazole, propiconazole, prochloraz, prothioconazole, tebuconazole, triadimefon, triadimenol, triflumizol, triticonazole, flutriafol;
F.6 dicarboximides such as iprodion, myclozolin, procymidon, vinclozolin;
F.7 dithiocarbamates such as ferbam, nabam, maneb, mancozeb, metam, metiram, propineb, polycarbamate, thiram, ziram, zineb;
F.8 heterocyclic compounds such as anilazine, benomyl, boscalid, carbendazim, carboxin, oxycarboxin, cyazofamid, dazomet, dithianon, famoxadon, fenamidon, fenarimol, fuberidazole, flutolanil, furametpyr, isoprothiolane, mepronil, nuarimol, probenazole, proquinazid, pyrifenox, pyroquilon, quinoxyfen, silthiofam, thiabendazole, thifluzamid, thiophanate-methyl, tiadinil, tricyclazole, triforine;
F.9 copper fungicides such as Bordeaux mixture, copper acetate, copper oxychloride, basic copper sulfate;
F.10 nitrophenyl derivatives such as binapacryl, dinocap, dinobuton, nitrophthalisopropyl;
F.11 phenylpyrroles such as fenpiclonil or fludioxonil;
F.12 strobilurins such as azoxystrobin, dimoxystrobin, fluoxastrobin, kresoxim-methyl, metominostrobin, orysastrobin, picoxystrobin or trifloxystrobin;
F.13 sulfenic acid derivatives such as captafol, captan, dichlofluanid, folpet, tolylfluanid;
F.14 cinnemamides and analogs such as dimethomorph, flumetover or flumorph;
F.15 sulfur, and other fungicides such as acibenzolar-S-methyl, benthiavalicarb, carpropamid, chlorothalonil, cyflufenamid, cymoxanil, dazomet, diclomezin, diclocymet, diethofencarb, edifenphos, ethaboxam, fenhexamid, fentin-acetate, fenoxanil, ferimzone, fluazinam, fosetyl, fosetyl-aluminum, iprovalicarb, hexachlorobenzene, metrafenon, pencycuron, propamocarb, phthalide, toloclofos-methyl, quintozene, zoxamid.

Compounds of formula II can also be formulated and combined with other active agents as described above for compounds of formula I.

### Applications

The animal pest, i.e. the insects, arachnids and nematodes, the plant, soil or water in which the plant is growing can be contacted with the present compound(s) I or composition(s) containing them by any application method known in the art. As such, “contacting” includes both direct contact (applying the compounds/compositions directly on the animal pest or plant—typically to the foliage, stem or roots of the plant) and indirect contact (applying the compounds/compositions to the locus of the animal pest or plant).

The compounds of formula I or the pesticidal compositions comprising them may be used to protect growing plants and crops from attack or infestation by animal pests, especially insects, acaridae or arachnids by contacting the plant/crop with a pesticidally effective amount of compounds of formula I. The term “crop” refers both to growing and harvested crops.

Moreover, animal pests may be controlled by contacting the target pest, its food supply, habitat, breeding ground or its locus with a pesticidally effective amount of compounds of formula I. As such, the application may be carried out before or after the infection of the locus, growing crops, or harvested crops by the pest.

The compounds of the invention can also be applied preventively to places at which occurrence of the pests is expected.

The compounds of formula I may be also used to protect growing plants from attack or infestation by pests by contacting the plant with a pesticidally effective amount of compounds of formula I. As such, “contacting” includes both direct contact (applying the compounds/compositions directly on the pest and/or plant—typically to the foliage, stem or roots of the plant) and indirect contact (applying the compounds/compositions to the locus of the pest and/or plant).

“Locus” means a habitat, breeding ground, plant, seed, soil, area, material or environment in which a pest or parasite is growing or may grow.

In general, “pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions such as desired pesticidal effect and duration, weather, target species, locus, mode of application, and the like.

The compounds of formula I are effective through both contact (via soil, glass, wall, bed net, carpet, plant parts or animal parts), and ingestion (bait, or plant part).

For use against ants, termites, wasps, flies, mosquitoes, crickets, or cockroaches, compounds of formula I are preferably used in a bait composition.

The bait can be a liquid, a solid or a semisolid preparation (e.g. a gel). Solid baits can be formed into various shapes and forms suitable to the respective application e.g. granules, blocks, sticks, disks. Liquid baits can be filled into various devices to ensure proper application, e.g. open containers, spray devices, droplet sources, or evaporation sources. Gels can be based on aqueous or oily matrices and can be formulated to particular necessities in terms of stickiness, moisture retention or aging characteristics.

The bait employed in the composition is a product, which is sufficiently attractive to incite insects such as ants, termites, wasps, flies, mosquitoes, crickets etc. or cock-roaches to eat it. The attractiveness can be manipulated by using feeding stimulants or sex pheromones. Food stimulants are chosen, for example, but not exclusively, from animal and/or plant proteins (meat-, fish- or blood meal, insect parts, egg yolk), from fats and oils of animal and/or plant origin, or mono-, oligo- or polyorganosaccharides, especially from sucrose, lactose, fructose, dextrose, glucose, starch, pectin or even molasses or honey. Fresh or decaying parts of fruits, crops, plants, animals, insects or specific parts thereof can also serve as a feeding stimulant. Sex pheromones are known to be more insect specific. Specific pheromones are described in the literature and are known to those skilled in the art.

Formulations of compounds of formula I as aerosols (e.g in spray cans), oil sprays or pump sprays are highly suitable for the non-professional user for controlling pests such as flies, fleas, ticks, mosquitoes or cockroaches. Aerosol recipes are preferably composed of the active compound, solvents such as lower alcohols (e.g. methanol, ethanol, propanol, butanol), ketones (e.g. acetone, methyl ethyl ketone), paraffin hydrocarbons (e.g. kerosenes) having boiling ranges of approximately 50 to 250° C., dimethylformamide, N-methylpyrrolidone, dimethyl sulphoxide, aromatic hydrocarbons such as toluene, xylene, water, furthermore auxiliaries such as emulsifiers such as sorbitol monooleate, oleyl ethoxylate having 3-7 mol of ethylene oxide, fatty alcohol ethoxylate, perfume oils such as ethereal oils, esters of medium fatty acids with lower alcohols, aromatic carbonyl compounds, if appropriate stabilizers such as sodium benzoate, amphoteric surfactants, lower epoxides, triethyl orthoformate and, if required, propellants such as propane, butane, nitrogen, compressed air, dimethyl ether, carbon dioxide, nitrous oxide, or mixtures of these gases.

The oil spray formulations differ from the aerosol recipes in that no propellants are used.

The compounds of formula I and its respective compositions can also be used in mosquito and fumigating coils, smoke cartridges, vaporizer plates or long-term vaporizers and also in moth papers, moth pads or other heat-independent vaporizer systems.

Methods to control infectious diseases transmitted by insects (e.g. malaria, dengue and yellow fever, lymphatic filariasis, and leishmaniasis) with compounds of formula I and its respective compositions also comprise treating surfaces of huts and houses, air spraying and impregnation of curtains, tents, clothing items, bed nets, tsetse-fly trap or the like. Insecticidal compositions for application to fibers, fabric, knitgoods, nonwovens, netting material or foils and tarpaulins preferably comprise a mixture including the insecticide, optionally a repellent and at least one binder. Suitable repellents for example are N,N-Diethyl-meta-toluamide (DEET), N,N-diethylphenylacetamide (DEPA), 1-(3-cyclohexan-1-yl-carbonyl)-2-methylpiperine, (2-hydroxymethylcyclohexyl)acetic acid lactone, 2-ethyl-1,3-hexandiol, indalone, Methylneodecanamide (MNDA), a pyrethroid not used for insect control such as {(+/−)-3-allyl-2-methyl-4-oxocyclopent-2-(+)-enyl-(+)-trans-chrysantemate (Esbiothrin), a repellent derived from or identical with plant extracts like limonene, eugenol, (+)-Eucamalol (1), (−)-1-epi-eucamalol or crude plant extracts from plants like Eucalyptus maculata, Vitex rotundifolia, Cymbopogan martinii, Cymbopogan citratus (lemon grass), Cymopogan nartdus (citronella). Suitable binders are selected for example from polymers and copolymers of vinyl esters of aliphatic acids (such as such as vinyl acetate and vinyl versatate), acrylic and methacrylic esters of alcohols, such as butyl acrylate, 2-ethylhexylacrylate, and methyl acrylate, mono- and di-ethylenically unsaturated hydrocarbons, such as styrene, and aliphatic diens, such as butadiene.

The impregnation of curtains and bednets is done in general by dipping the textile material into emulsions or dispersions of the insecticide or spraying them onto the nets.

The compounds of formula I and its compositions can be used for protecting wooden materials such as trees, board fences, sleepers, etc. and buildings such as houses, outhouses, factories, but also construction materials, furniture, leathers, fibers, vinyl articles, electric wires and cables etc. from ants and/or termites, and for controlling ants and termites from doing harm to crops or human being (e.g. when the pests invade into houses and public facilities). The compounds of formula I are applied not only to the surrounding soil surface or into the under-floor soil in order to protect wooden materials but it can also be applied to lumbered articles such as surfaces of the under-floor concrete, alcove posts, beams, plywoods, furniture, etc., wooden articles such as particle boards, half boards, etc. and vinyl articles such as coated electric wires, vinyl sheets, heat insulating material such as styrene foams, etc. In case of application against ants doing harm to crops or human beings, the ant controller of the present invention is applied to the crops or the surrounding soil, or is directly applied to the nest of ants or the like.

In the case of soil treatment or of application to the pests dwelling place or nest, the quantity of active ingredient ranges from 0.0001 to 500 g per 100 m², preferably from 0.001 to 20 g per 100 m².

Customary application rates in the protection of materials are, for example, from 0.01 g to 1000 g of active compound per m² treated material, desirably from 0.1 g to 50 g per m².

Insecticidal compositions for use in the impregnation of materials typically contain from 0.001 to 95 weight %, preferably from 0.1 to 45 weight %, and more preferably from 1 to 25 weight % of at least one repellent and/or insecticide.

For use in bait compositions, the typical content of active ingredient is from 0.001 weight % to 15 weight %, desirably from 0.001 weight % to 5% weight % of active compound.

For use in spray compositions, the content of active ingredient is from 0.001 to 80 weights %, preferably from 0.01 to 50 weight % and most preferably from 0.01 to 15 weight %.

For use in treating crop plants, the rate of application of the active ingredients of this invention may be in the range of 0.1 g to 4000 g per hectare, desirably from 25 g to 600 g per hectare, more desirably from 50 g to 500 g per hectare.

Compounds of formula II can also be applied and used as described above for compounds of formula I.

### Seed Treatment

The compounds of formula I are also suitable for the treatment of seeds in order to protect the seed from insect pest, in particular from soil-living insect pests and the resulting plant's roots and shoots against soil pests and foliar insects.

The compounds of formula I are particularly useful for the protection of the seed from soil pests and the resulting plant's roots and shoots against soil pests and foliar insects. The protection of the resulting plant's roots and shoots is preferred. More preferred is the protection of resulting plant's shoots from piercing and sucking insects, wherein the protection from aphids is most preferred.

The present invention therefore comprises a method for the protection of seeds from insects, in particular from soil insects and of the seedlings' roots and shoots from insects, in particular from soil and foliar insects, said method comprising contacting the seeds before sowing and/or after pregermination with a compound of the general formula I or a salt thereof. Particularly preferred is a method, wherein the plant's roots and shoots are protected, more preferably a method, wherein the plants shoots are protected form piercing and sucking insects, most preferably aa method, wherein the plants shoots are protected from aphids.

The term seed embraces seeds and plant propagules of all kinds including but not limited to true seeds, seed pieces, suckers, corms, bulbs, fruit, tubers, grains, cuttings, cut shoots and the like and means in a preferred embodiment true seeds.

The term seed treatment comprises all suitable seed treatment techniques known in the art, such as seed dressing, seed coating, seed dusting, seed soaking and seed pelleting.

The present invention also comprises seeds coated with or containing the active compound.

The term “coated with and/or containing” generally signifies that the active ingredient is for the most part on the surface of the propagation product at the time of application, although a greater or lesser part of the ingredient may penetrate into the propagation product, depending on the method of application. When the said propagation product is (re)planted, it may absorb the active ingredient.

Suitable seed is seed of cereals, root crops, oil crops, vegetables, spices, ornamentals, for example seed of durum and other wheat, barley, oats, rye, maize (fodder maize and sugar maize/sweet and field corn), soybeans, oil crops, crucifers, cotton, sunflowers, bananas, rice, oilseed rape, turnip rape, sugarbeet, fodder beet, eggplants, potatoes, grass, lawn, turf, fodder grass, tomatoes, leeks, pumpkin/squash, cabbage, iceberg lettuce, pepper, cucumbers, melons, Brassica species, melons, beans, peas, garlic, onions, carrots, tuberous plants such as potatoes, sugar cane, tobacco, grapes, petunias, geranium/pelargoniums, pansies and impatiens.

In addition, the active compound may also be used for the treatment seeds from plants, which tolerate the action of herbicides or fungicides or insecticides owing to breeding, including genetic engineering methods.

For example, the active compound can be employed in treatment of seeds from plants, which are resistant to herbicides from the group consisting of the sulfonylureas, imidazolinones, glufosinate-ammonium or glyphosate-isopropylammonium and analogous active substances (see for example, EP-A-0242236, EP-A-242246) (WO 92/00377) (EP-A-0257993, U.S. Pat. No. 5,013,659) or in transgenic crop plants, for example cotton, with the capability of producing Bacillus thuringiensis toxins (Bt toxins) which make the plants resistant to certain pests (EP-A-0142924, EP-A-0193259),

Furthermore, the active compound can be used also for the treatment of seeds from plants, which have modified characteristics in comparison with existing plants consist, which can be generated for example by traditional breeding methods and/or the generation of mutants, or by recombinant procedures). For example, a number of cases have been described of recombinant modifications of crop plants for the purpose of modifying the starch synthesized in the plants (e.g. WO 92/11376, WO 92/14827, WO 91/19806) or of transgenic crop plants having a modified fatty acid composition (WO 91/13972).

The seed treatment application of the active compound is carried out by spraying or by dusting the seeds before sowing of the plants and before emergence of the plants.

Compositions which are especially useful for seed treatment are e.g.:

| | |
|---|---|
| A | Soluble concentrates (SL, LS) |
| D | Emulsions (EW, EO, ES) |
| E | Suspensions (SC, OD, FS) |
| F | Water-dispersible granules and water-soluble granules (WG, SG) |
| G | Water-dispersible powders and water-soluble powders (WP, SP, WS) |
| H | Gel-Formulations (GF) |
| I | Dustable powders (DP, DS) |

Conventional seed treatment formulations include for example flowable concentrates FS, solutions LS, powders for dry treatment DS, water dispersible powders for slurry treatment WS, water-soluble powders SS and emulsion ES and EC and gel formulation GF. These formulations can be applied to the seed diluted or undiluted. Application to the seeds is carried out before sowing, either directly on the seeds or after having pregerminated the latter

In a preferred embodiment a FS formulation is used for seed treatment. Typically, a FS formulation may comprise 1-800 g/l of active ingredient, 1-200 g/l Surfactant, 0 to 200 g/l antifreezing agent, 0 to 400 g/l of binder, 0 to 200 g/l of a pigment and up to 1 liter of a solvent, preferably water.

Especially preferred FS formulations of compounds of formula I for seed treatment usually comprise from 0.1 to 80% by weight (1 to 800 g/l) of the active ingredient, from 0.1 to 20% by weight (1 to 200 g/l) of at least one surfactant, e.g. 0.05 to 5% by weight of a wetter and from 0.5 to 15% by weight of a dispersing agent, up to 20% by weight, e.g. from 5 to 20% of an anti-freeze agent, from 0 to 15% by weight, e.g. 1 to 15% by weight of a pigment and/or a dye, from 0 to 40% by weight, e.g. 1 to 40% by weight of a binder (sticker/adhesion agent), optionally up to 5% by weight, e.g. from 0.1 to 5% by weight of a thickener, optionally from 0.1 to 2% of an anti-foam agent, and optionally a preservative such as a biocide, antioxidant or the like, e.g. in an amount from 0.01 to 1% by weight and a filler/vehicle up to 100% by weight.

Seed Treatment formulations may additionally also comprise binders and optionally colorants.

Binders can be added to improve the adhesion of the active materials on the seeds after treatment. Suitable binders are homo- and copolymers from alkylene oxides like ethylene oxide or propylene oxide, polyvinylacetate, polyvinylalcohols, polyvinylpyrrolidones, and copolymers thereof, ethylene-vinyl acetate copolymers, acrylic homo- and copolymers, polyethyleneamines, polyethyleneamides and polyethyleneimines, polysaccharides like celluloses, tylose and starch, polyolefin homo- and copolymers like olefin/maleic anhydride copolymers, polyurethanes, polyesters, polystyrene homo and copolymers

Optionally, also colorants can be included in the formulation. Suitable colorants or dyes for seed treatment formulations are Rhodamin B, C.I. Pigment Red 112, C.I. Solvent Red 1, pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Examples of a gelling agent is carrageen (Satiagel®)

In the treatment of seed, the application rates of the compounds I are generally from 0.1 g to 10 kg per 100 kg of seed, preferably from 1 g to 5 kg per 100 kg of seed, more preferably from 1 g to 1000 g per 100 kg of seed and in particular from 1 g to 200 g per 100 kg of seed.

The invention therefore also relates to seed comprising a compound of the formula I, or an agriculturally useful salt of I, as defined herein. The amount of the compound I or the agriculturally useful salt thereof will in general vary from 0.1 g to 10 kg per 100 kg of seed, preferably from 1 g to 5 kg per 100 kg of seed, in particular from 1 g to 1000 g per 100 kg of seed. For specific crops such as lettuce the rate can be higher.

Compounds of formula II can also be used for seed treatment purposes as described above for compounds of formula I.

### Animal Health

The compounds of formula I or the enantiomers or veterinarily acceptable salts thereof are in particular also suitable for being used for combating parasites in and on animals.

An object of the present invention is therefore also to provide new methods to control parasites in and on animals. Another object of the invention is to provide safer pesticides for animals. Another object of the invention is further to provide pesticides for animals that may be used in lower doses than existing pesticides. And another object of the invention is to provide pesticides for animals, which provide a long residual control of the parasites.

The invention also relates to compositions containing a parasiticidally effective amount of compounds of formula I or the enantiomers or veterinarily acceptable salts thereof and an acceptable carrier, for combating parasites in and on animals.

The present invention also provides a method for treating, controlling, preventing and protecting animals against infestation and infection by parasites, which comprises orally, topically or parenterally administering or applying to the animals a parasiticidally effective amount of a compound of formula I or the enantiomers or veterinarily acceptable salts thereof or a composition comprising it.

The invention also provides a process for the preparation of a composition for treating, controlling, preventing or protecting animals against infestation or infection by parasites which comprises a parasiticidally effective amount of a compound of formula I or the enantiomers or veterinarily acceptable salts thereof or a composition comprising it.

Activity of compounds against agricultural pests does not suggest their suitability for control of endo- and ectoparasites in and on animals which requires, for example, low, non-emetic dosages in the case of oral application, metabolic compatibility with the animal, low toxicity, and a safe handling.

Surprisingly it has now been found that compounds of formula I are suitable for combating endo- and ectoparasites in and on animals.

Compounds of formula I or the enantiomers or veterinarily acceptable salts thereof and compositions comprising them are preferably used for controlling and preventing infestations and infections animals including warm-blooded animals (including humans) and fish. They are for example suitable for controlling and preventing infestations and infections in mammals such as cattle, sheep, swine, camels, deer, horses, pigs, poultry, rabbits, goats, dogs and cats, water buffalo, donkeys, fallow deer and reindeer, and also in fur-bearing animals such as mink, chinchilla and raccoon, birds such as hens, geese, turkeys and ducks and fish such as fresh- and salt-water fish such as trout, carp and eels.

Compounds of formula I or the enantiomers or veterinarily acceptable salts thereof and compositions comprising them are preferably used for controlling and preventing infestations and infections in domestic animals, such as dogs or cats.

Infestations in warm-blooded animals and fish include, but are not limited to, lice, biting lice, ticks, nasal bots, keds, biting flies, muscoid flies, flies, myiasitic fly larvae, chiggers, gnats, mosquitoes and fleas.

The compounds of formula I or the enantiomers or veterinarily acceptable salts thereof and compositions comprising them are suitable for systemic and/or non-systemic control of ecto- and/or endoparasites. They are active against all or some stages of development.

The compounds of formula I are especially useful for combating ectoparasites.

The compounds of formula I are especially useful for combating parasites of the following orders and species, respectively:

fleas (Siphonaptera), e.g. Ctenocephalides felis, Ctenocephalides canis, Xenopsylla cheopis, Pulex irritans, Tunga penetrans, and Nosopsyllus fasciatus,

cockroaches (Blattaria-Blattodea), e.g. Blattella germanica, Blattella asahinae, Periplaneta americana, Periplaneta japonica, Periplaneta brunnea, Periplaneta fuligginosa, Periplaneta australasiae, and Blatta orientalis,

flies, mosquitoes (Diptera), e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Chrysomya bezziana, Chrysomya hominivorax, Chrysomya macellaria, Chrysops discalis, Chrysops silacea, Chrysops atlanticus, Cochliomyia hominivorax, Cordylobia anthropophaga, Culicoides furens, Culex pipiens, Culex nigripalpus, Culex quinquefasciatus, Culex tarsalis, Culiseta inornata, Culiseta melanura, Dermatobia hominis, Fannia canicularis, Gasterophilus intestinalis, Glossina morsitans, Glossina palpalis, Glossina fuscipes, Glossina tachinoides, Haematobia irritans, Haplodiplosis equestris, Hippelates spp., Hypoderma lineata, Leptoconops torrens, Lucilia caprina, Lucilia cuprina, Lucilia sericata, Lycoria pectoralis, Mansonia spp., Musca domestica, Muscina stabulans, Oestrus ovis, Phlebotomus argentipes, Psorophora columbiae, Psorophora discolor, Prosimulium mixtum, Sarcophaga haemorrhoidalis, Sarcophaga sp., Simulium vittatum, Stomoxys calcitrans, Tabanus bovinus, Tabanus atratus, Tabanus lineola, and Tabanus similis,

lice (Phthiraptera), e.g. Pediculus humanus capitis, Pediculus humanus corporis, Pthirus pubis, Haematopinus eurysternus, Haematopinus suis, Linognathus vituli, Bovicola bovis, Menopon gallinae, Menacanthus stramineus and Solenopotes capillatus.

ticks and parasitic mites (Parasitiformes): ticks (Ixodida), e.g. Ixodes scapularis, Ixodes holocyclus, Ixodes pacificus, Rhiphicephalus sanguineus, Dermacentor andersoni, Dermacentor variabilis, Amblyomma americanum, Ambryomma maculatum, Ornithodorus hermsi, Ornithodorus turicata and parasitic mites (Mesostigmata), e.g. Ornithonyssus bacoti and Dermanyssus gallinae,

Actinedida (Prostigmata) und Acaridida (Astigmata) e.g. Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., and Laminosioptes spp,

Bugs (Heteropterida): Cimex lectularius, Cimex hemipterus, Reduvius senilis, Triatoma spp., Rhodnius ssp., Panstrongylus ssp. and Arilus critatus,

Anoplurida, e.g. Haematopinus spp., Linognathus spp., Pediculus spp., Phtirus spp., and Solenopotes spp,

Mallophagida (suborders Arnblycerina and Ischnocerina), e.g. Trimenopon spp., Menopon spp., Trinoton spp., Bovicola spp., Werneckiella spp., Lepikentron spp., Trichodectes spp., and Felicola spp,

### Roundworms Nematoda:

Wipeworms and Trichinosis (Trichosyringida), e.g. Trichinellidae (Trichinella spp.), (Trichuridae) Trichuris spp., Capillaria spp,

Rhabditida, e.g. Rhabditis spp, Strongyloides spp., Helicephalobus spp,

Strongylida, e.g. Strongylus spp., Ancylostoma spp., Necator americanus, Bunostomum spp. (Hookworm), Trichostrongylus spp., Haemonchus contortus, Ostertagia spp., Cooperia spp., Nematodirus spp., Dictyocaulus spp., Cyathostoma spp., Oesophagostomum spp., Stephanurus dentatus, Ollulanus spp., Chabertia spp., Stephanurus dentatus, Syngamus trachea, Ancylostoma spp., Uncinaria spp., Globocephalus spp., Necator spp., Metastrongylus spp., Muellerius capillaris, Protostrongylus spp., Angiostrongylus spp., Parelaphostrongylus spp. Aleurostrongylus abstrusus, and Dioctophyma renale,

Intestinal roundworms (Ascaridida), e.g. Ascaris lumbricoides, Ascaris suum, Ascaridia galli, Parascaris equorum, Enterobius vermicularis (Threadworm), Toxocara canis, Toxascaris leonine, Skrjabinema spp., and Oxyuris equi,

Camallanida, e.g. Dracunculus medinensis (guinea worm)

Spirurida, e.g. Thelazia spp. Wuchereria spp., Brugia spp., Onchocerca spp., Dirofilari spp.a, Dipetalonema spp., Setaria spp., Elaeophora spp., Spirocerca lupi, and Habronema spp.,

Thorny headed worms (Acanthocephala), e.g. Acanthocephalus spp., Macracanthorhynchus hirudinaceus and Oncicola spp,

Planarians (Plathelminthes):

Flukes (Trematoda), e.g. Faciola spp., Fascioloides magna, Paragonimus spp., Dicrocoelium spp., Fasciolopsis buski, Clonorchis sinensis, Schistosoma spp., Trichobilharzia spp., Alaria alata, Paragonimus spp., and Nanocyetes spp,

Cercomeromorpha, in particular Cestoda (Tapeworms), e.g. Diphyllobothrium spp., Tenia spp., Echinococcus spp., Dipylidium caninum, Multiceps spp., Hymenolepis spp., Mesocestoides spp., Vampirolepis spp., Moniezia spp., Anoplocephala spp., Sirometra spp., Anoplocephala spp., and Hymenolepis spp.

The compounds of formula I and compositions containing them are particularly useful for the control of pests from the orders Diptera, Siphonaptera and Ixodida.

Moreover, the use of the compounds of formula I and compositions containing them for combating mosquitoes is especially preferred.

The use of the compounds of formula I and compositions containing them for combating flies is a further preferred embodiment of the present invention.

Furthermore, the use of the compounds of formula I and compositions containing them for combating fleas is especially preferred.

The use of the compounds of formula I and compositions containing them for combating ticks is a further preferred embodiment of the present invention.

The compounds of formula I also are especially useful for combating endoparasites (roundworms nematoda, thorny headed worms and planarians).

Administration can be carried out both prophylactically and therapeutically.

Administration of the active compounds is carried out directly or in the form of suitable preparations, orally, topically/dermally or parenterally.

For oral administration to warm-blooded animals, the formula I compounds may be formulated as animal feeds, animal feed premixes, animal feed concentrates, pills, solutions, pastes, suspensions, drenches, gels, tablets, boluses and capsules. In addition, the formula I compounds may be administered to the animals in their drinking water. For oral administration, the dosage form chosen should provide the animal with 0.01 mg/kg to 100 mg/kg of animal body weight per day of the formula I compound, preferably with 0.5 mg/kg to 100 mg/kg of animal body weight per day.

Alternatively, the formula I compounds may be administered to animals parenterally, for example, by intraruminal, intramuscular, intravenous or subcutaneous injection. The formula I compounds may be dispersed or dissolved in a physiologically acceptable carrier for subcutaneous injection. Alternatively, the formula I compounds may be formulated into an implant for subcutaneous administration. In addition the formula I compound may be transdermally administered to animals. For parenteral administration, the dosage form chosen should provide the animal with 0.01 mg/kg to 100 mg/kg of animal body weight per day of the formula I compound.

The formula I compounds may also be applied topically to the animals in the form of dips, dusts, powders, collars, medallions, sprays, shampoos, spot-on and pour-on formulations and in ointments or oil-in-water or water-in-oil emulsions. For topical application, dips and sprays usually contain 0.5 ppm to 5,000 ppm and preferably 1 ppm to 3,000 ppm of the formula I compound. In addition, the formula I compounds may be formulated as ear tags for animals, particularly quadrupeds such as cattle and sheep.

Suitable preparations are:
Solutions such as oral solutions, concentrates for oral administration after dilution, solutions for use on the skin or in body cavities, pouring-on formulations, gels;
Emulsions and suspensions for oral or dermal administration; semi-solid preparations;
Formulations in which the active compound is processed in an ointment base or in an oil-in-water or water-in-oil emulsion base;
Solid preparations such as powders, premixes or concentrates, granules, pellets, tablets, boluses, capsules; aerosols and inhalants, and active compound-containing shaped articles.

Compositions suitable for injection are prepared by dissolving the active ingredient in a suitable solvent and optionally adding further ingredients such as acids, bases, buffer salts, preservatives, and solubilizers. The solutions are filtered and filled sterile.

Suitable solvents are physiologically tolerable solvents such as water, alkanols such as ethanol, butanol, benzyl alcohol, glycerol, propylene glycol, polyethylene glycols, N-methyl-pyrrolidone, 2-pyrrolidone, and mixtures thereof.

The active compounds can optionally be dissolved in physiologically tolerable vegetable or synthetic oils which are suitable for injection.

Suitable solubilizers are solvents which promote the dissolution of the active compound in the main solvent or prevent its precipitation. Examples are polyvinylpyrrolidone, polyvinyl alcohol, polyoxyethylated castor oil, and polyoxyethylated sorbitan ester.

Suitable preservatives are benzyl alcohol, trichlorobutanol, p-hydroxybenzoic acid esters, and n-butanol.

Oral solutions are administered directly. Concentrates are administered orally after prior dilution to the use concentration. Oral solutions and concentrates are prepared according to the state of the art and as described above for injection solutions, sterile procedures not being necessary.

Solutions for use on the skin are trickled on, spread on, rubbed in, sprinkled on or sprayed on.

Solutions for use on the skin are prepared according to the state of the art and according to what is described above for injection solutions, sterile procedures not being necessary.

Further suitable solvents are polypropylene glycol, phenyl ethanol, phenoxy ethanol, ester such as ethyl or butyl acetate, benzyl benzoate, ethers such as alkyleneglycol alkylether, e.g. dipropylenglycol monomethylether, ketons such as acetone, methylethylketone, aromatic hydrocarbons, vegetable and synthetic oils, dimethylformamide, dimethylacetamide, transcutol, solketal, propylencarbonate, and mixtures thereof.

It may be advantageous to add thickeners during preparation. Suitable thickeners are inorganic thickeners such as bentonites, colloidal silicic acid, aluminium monostearate, organic thickeners such as cellulose derivatives, polyvinyl alcohols and their copolymers, acrylates and methacrylates.

Gels are applied to or spread on the skin or introduced into body cavities. Gels are prepared by treating solutions which have been prepared as described in the case of the injection solutions with sufficient thickener that a clear material having an ointment-like consistency results. The thickeners employed are the thickeners given above.

Pour-on formulations are poured or sprayed onto limited areas of the skin, the active compound penetrating the skin and acting systemically.

Pour-on formulations are prepared by dissolving, suspending or emulsifying the active compound in suitable skin-compatible solvents or solvent mixtures. If appropriate, other auxiliaries such as colorants, bioabsorption-promoting substances, antioxidants, light stabilizers, adhesives are added.

Suitable solvents which are: water, alkanols, glycols, polyethylene glycols, polypropylene glycols, glycerol, aromatic alcohols such as benzyl alcohol, phenylethanol, phenoxyethanol, esters such as ethyl acetate, butyl acetate, benzyl benzoate, ethers such as alkylene glycol alkyl ethers such as dipropylene glycol monomethyl ether, diethylene glycol mono-butyl ether, ketones such as acetone, methyl ethyl ketone, cyclic carbonates such as propylene carbonate, ethylene carbonate, aromatic and/or aliphatic hydrocarbons, vegetable or synthetic oils, DMF, dimethylacetamide, n-alkylpyrrolidones such as methylpyrrolidone, n-butylpyrrolidone or n-octylpyrrolidone, N-methylpyrrolidone, 2-pyrrolidone, 2,2-dimethyl-4-oxy-methylene-1,3-diox-olane and glycerol formal.

Suitable colorants are all colorants permitted for use on animals and which can be dissolved or suspended.

Suitable absorption-promoting substances are, for example, DMSO, spreading oils such as isopropyl myristate, dipropylene glycol pelargonate, silicone oils and copolymers thereof with polyethers, fatty acid esters, triglycerides, fatty alcohols.

Suitable antioxidants are sulfites or metabisulfites such as potassium metabisulfite, ascorbic acid, butylhydroxytoluene, butylhydroxyanisole, tocopherol.

Suitable light stabilizers are, for example, novantisolic acid.

Suitable adhesives are, for example, cellulose derivatives, starch derivatives, polyacrylates, natural polymers such as alginates, gelatin.

Emulsions can be administered orally, dermally or as injections.

Emulsions are either of the water-in-oil type or of the oil-in-water type.

They are prepared by dissolving the active compound either in the hydrophobic or in the hydrophilic phase and homogenizing this with the solvent of the other phase with the aid of suitable emulsifiers and, if appropriate, other auxiliaries such as colorants, absorption-promoting substances, preservatives, antioxidants, light stabilizers, viscosity-enhancing substances.

Suitable hydrophobic phases (oils) are:

liquid paraffins, silicone oils, natural vegetable oils such as sesame oil, almond oil, castor oil, synthetic triglycerides such as caprylic/capric biglyceride, triglyceride mixture with vegetable fatty acids of the chain length C₈-C₁₂ or other specially selected natural fatty acids, partial glyceride mixtures of saturated or unsaturated fatty acids possibly also containing hydroxyl groups, mono- and diglycerides of the C₈-C₁₀ fatty acids, fatty acid esters such as ethyl stearate, di-n-butyryl adipate, hexyl laurate, dipropylene glycol perlargonate, esters of a branched fatty acid of medium chain length with saturated fatty alcohols of chain length C₁₆-C₁₈, isopropyl myristate, isopropyl palmitate, caprylic/capric acid esters of saturated fatty alcohols of chain length C₁₂-C₁₈, isopropyl stearate, oleyl oleate, decyl oleate, ethyl oleate, ethyl lactate, waxy fatty acid esters such as synthetic duck coccygeal gland fat, dibutyl phthalate, diisopropyl adipate, and ester mixtures related to the latter, fatty alcohols such as isotridecyl alcohol, 2-octyldodecanol, cetylstearyl alcohol, oleyl alcohol, and fatty acids such as oleic acid and mixtures thereof.

Suitable hydrophilic phases are: water, alcohols such as propylene glycol, glycerol, sorbitol and mixtures thereof.

Suitable emulsifiers are:

non-ionic surfactants, e.g. polyethoxylated castor oil, polyethoxylated sorbitan monooleate, sorbitan monostearate, glycerol monostearate, polyoxyethyl stearate, alkylphenol polyglycol ether;

ampholytic surfactants such as di-sodium N-lauryl-p-iminodipropionate or lecithin; anionic surfactants, such as sodium lauryl sulfate, fatty alcohol ether sulfates, mono/dialkyl polyglycol ether orthophosphoric acid ester monoethanolamine salt; cation-active surfactants, such as cetyltrimethylammonium chloride.

Suitable further auxiliaries are: substances which enhance the viscosity and stabilize the emulsion, such as carboxymethylcellulose, methylcellulose and other cellulose and starch derivatives, polyacrylates, alginates, gelatin, gum arabic, polyvinylpyrrolidone, polyvinyl alcohol, copolymers of methyl vinyl ether and maleic anhydride, polyethylene glycols, waxes, colloidal silicic acid or mixtures of the substances mentioned.

Suspensions can be administered orally or topically/dermally. They are prepared by suspending the active compound in a suspending agent, if appropriate with addition of other auxiliaries such as wetting agents, colorants, bioabsorption-promoting substances, preservatives, antioxidants, light stabilizers.

Liquid suspending agents are all homogeneous solvents and solvent mixtures.

Suitable wetting agents (dispersants) are the emulsifiers given above.

Other auxiliaries which may be mentioned are those given above.

Semi-solid preparations can be administered orally or topically/dermally. They differ from the suspensions and emulsions described above only by their higher viscosity.

For the production of solid preparations, the active compound is mixed with suitable excipients, if appropriate with addition of auxiliaries, and brought into the desired form.

Suitable excipients are all physiologically tolerable solid inert substances. Those used are inorganic and organic substances. Inorganic substances are, for example, sodium chloride, carbonates such as calcium carbonate, hydrogencarbonates, aluminium oxides, titanium oxide, silicic acids, argillaceous earths, precipitated or colloidal silica, or phosphates. Organic substances are, for example, sugar, cellulose, foodstuffs and feeds such as milk powder, animal meal, grain meals and shreds, starches.

Suitable auxiliaries are preservatives, antioxidants, and/or colorants which have been mentioned above.

Other suitable auxiliaries are lubricants and glidants such as magnesium stearate, stearic acid, talc, bentonites, disintegration-promoting substances such as starch or crosslinked polyvinylpyrrolidone, binders such as starch, gelatin or linear polyvinylpyrrolidone, and dry binders such as microcrystalline cellulose.

In general, “parasiticidally effective amount” means the amount of active ingredient needed to achieve an observable effect on growth, including the effects of necrosis, death, retardation, prevention, and removal, destruction, or otherwise diminishing the occurrence and activity of the target organism. The parasiticidally effective amount can vary for the various compounds/compositions used in the invention. A parasiticidally effective amount of the compositions will also vary according to the prevailing conditions such as desired parasiticidal effect and duration, target species, mode of application, and the like.

The compositions which can be used in the invention can comprise generally from about 0.001 to 95% of the compound of formula I.

Generally it is favorable to apply the compounds of formula I in total amounts of 0.5 mg/kg to 100 mg/kg per day, preferably 1 mg/kg to 50 mg/kg per day.

Ready-to-use preparations contain the compounds acting against parasites, preferably ectoparasites, in concentrations of 10 ppm to 80 percent by weight, preferably from 0.1 to 65 percent by weight, more preferably from 1 to 50 percent by weight, most preferably from 5 to 40 percent by weight.

Preparations which are diluted before use contain the compounds acting against ectoparasites in concentrations of 0.5 to 90 percent by weight, preferably of 1 to 50 percent by weight.

Furthermore, the preparations comprise the compounds of formula I against endoparasites in concentrations of 10 ppm to 2 percent by weight, preferably of 0.05 to 0.9 percent by weight, very particularly preferably of 0.005 to 0.25 percent by weight.

In a preferred embodiment of the present invention, the compositions comprising the compounds of formula I them are applied dermally/topically.

In a further preferred embodiment, the topical application is conducted in the form of compound-containing shaped articles such as collars, medallions, ear tags, bands for fixing at body parts, and adhesive strips and foils.

Generally it is favorable to apply solid formulations which release compounds of formula I in total amounts of 10 mg/kg to 300 mg/kg, preferably 20 mg/kg to 200 mg/kg, most preferably 25 mg/kg to 160 mg/kg body weight of the treated animal in the course of three weeks.

For the preparation of the shaped articles, thermoplastic and flexible plastics as well as elastomers and thermoplastic elastomers are used. Suitable plastics and elastomers are polyvinyl resins, polyurethane, polyacrylate, epoxy resins, cellulose, cellulose derivatives, polyamides and polyester which are sufficiently compatible with the compounds of formula I. A detailed list of plastics and elastomers as well as preparation procedures for the shaped articles is given e.g. in WO 03/086075.

Compounds of formula II can be used for animal health purposes as described above as for compounds of formula I.

The present invention is now illustrated in further detail by the following examples.

### PREPARATION EXAMPLES

In the following the preparation of compounds of formula I and intermediate compounds of formula II are described.

### P.1. Preparation of (4,5-Dihydro-thiazol-2-yl)-[1-(5-methyl-thiophen-2-yl)-2-pyridin-4-yl-ethyl]-amine (compound C.I.1 of table II)

### Step 1. Preparation of (Rₛ)-2-methyl-propane-2-sulfinic acid 5-methyl-thiophen-2-yl-methylene amide

To a solution of 4.37 g 5-methyl-thiophene-2-carbaldehyde and 4.00 g (R_{S})-(+)-2-methyl-2-propanesulfinamide (CAS [196929-78-9], NetChem, Catalog Number 497401-A) in 30 ml tetrahydrofuran (THF) was added 46.9 g titanium tetraisopropoxide at room temperature and stirring continued overnight. The reaction mixture was poured via stirring into 400 ml of water and the precipitate removed via filtration through diatomaceous earth. The filtrate was extracted with ethyl acetate and the organic phases were thoroughly washed with sodium bisulfite solution (20 wt % in water) and water, and were dried over sodium sulfate. Evaporation of the solvents gave the pure product (7.35 g) as a yellowish powder.

¹H-NMR (CDCl₃): δ=1.25 (s), 2.55 (s), 6.80 (d), 7.35 (d), 8.55 (s).

### Step 2. Preparation of (R_{S})-2-methyl-propane-2-sulfinic acid [1-(5-methyl-thiophen-2-yl)-2-pyridin-4-yl-ethyl]-amide

A solution of 0.79 g 4-methylpyridine in 15 ml of THF was treated at 0° C. with a freshly prepared solution of 2,2,6,6-tetramethyl-piperidine lithium (LiTMP) drop by drop over a period of 1.5 hours by means of a syringe pump. LiTMP was prepared by adding 8.51 mmol n-butyl lithium to a solution of 1.2 g 2,2,6,6-tetramethyl-piperidine in 15 ml of THF at room temperature.

The solution was stirred for one hour at this temperature and than a solution of 1.9 g of (Rₛ)-2-methyl-propane-2-sulfinic acid 5-methyl-thiophen-2-ylmethylene amide in 15 ml THF was added within 45 minutes (by means of a syringe pump). The reaction mixture was allowed to reach room temperature and stirring was continued for 2 hours. Aqueous potassium carbonate solution (5 wt %) was added, extracted with diethyl ether and the organic phases dried over sodium sulfate. Evaporation of the solvent yielded a residue that was purified by column chromatography on silica gel yielding the title compound as a mixture of diastereomeres in a 3:1 ratio (1.85 g).

¹H-NMR (CDCl₃): major isomer: δ=1.15 (s), 2.45 (s), 3.05 (mc), 3.30 (mc), 3.55 (mc), 4.85 (mc), 6.50 (d), 6.65 (d), 7.0 (mc), 8.45 (mc); minor isomer: δ=1.20 (s), 2.45 (s), 3.15 (mc), 3.55 (mc), 4.9 (mc), 6.50 (d), 6.10 (d), 7.0 (mc), 8.45 (mc).

### Step 3. Preparation of 1-(5-methyl-thiophen-2-yl)-2-pyridin-4-yl-ethylamine

A solution of 1.68 g (R_{S})-2-methyl-propane-2-sulfinic acid [1-(5-methyl-thiophen-2-yl)-2-pyridin-4-yl-ethyl]-amide in 10 ml methanol was treated at room temperature with 10 ml of a solution of hydrogen chloride in dioxane (4 N) and stirring continued overnight. The mixture was concentrated in vacuo, the reminder treated with potassium carbonate solution (5 wt % in water) until pH was around 8-9 and then extracted with ethyl acetate. The organic phases were washed with water, dried over sodium sulfate and solvents evaporated. This yielded the product (0.9 g) as a yellowish oil).

¹H-NMR (CDCl₃): δ=2.45 (s), 2.90 (dd), 3.05 (dd), 4.40 (mc), 6.50 (d), 6.60 (d), 7.10 (dd), 8.50 (d).

### Step 4. Preparation of acetic acid 2-{3-[1-(5-methyl-thiophen-2-yl)-2-pyridin-4-yl-ethyl]-thioureido}-ethyl ester

A solution of 0.83 g 1-(5-methyl-thiophen-2-yl)-2-pyridin-4-yl-ethylamine in 40 ml of THF was treated with 0.55 g acetic acid 2-isothiocyanato-ethylester at room temperature. The reaction mixture was concentrated in vacuo and purified by column chromatography to yield 1.05 g acetic acid 2-{3-[1-(5-methyl-thiophen-2-yl)-2-pyridin-4-yl-ethyl]-thioureido}-ethyl ester as a viscous oil.

¹H-NMR (CDCl₃): δ=2.0 (s), 2.45 (s), 3.15 (dd), 3.40 (dd), 3.75 (mc), 4.15 (mc), 5.80 (mc), 6.50 (d), 6.1-6.2 (m), 7.10 (d), 8.40 (d).

### Step 5. Preparation of 1-(2-hydroxy-ethyl)-3-[1-(5-methyl-thiophen-2-yl)-2-pyridin-4-yl-ethyl]-thiourea

A solution of 0.90 g 2-{3-[1-(5-methyl-thiophen-2-yl)-2-pyridin-4-yl-ethyl]-thioureido}-ethyl ester in 20 ml of THF was treated at room temperature with a solution of 0.12 g lithium hydroxide in 20 ml of water, and stirring was continued for 4 h. The mixture was poured into 300 ml of ethyl acetate, washed with water and dried of sodium sulfate to yield 0.70 g of a viscous oil.

¹H-NMR (d6-DMSO): δ=2.35 (s), 3.1-3.5 (m), 4.8 (s), 5.9 (s), 6.6 (s), 6.8 (s), 7.25 (d), 7.4 (s), 7.95 (d), 8.45 (d).

### Step 6. Preparation of (4,5-Dihydro-thiazol-2-yl)-[1-(5-methyl-thiophen-2-yl)-2-pyridin-4-yl-ethyl]-amine

To a solution of 1-(2-hydroxy-ethyl)-3-[1-(5-methyl-thiophen-2-yl)-2-pyridin-4-yl-ethyl]-thiourea (0.50 g) and 0.44 g diisopropylethylamine in 15 ml of propionitrile was added at room temperature 0.83 g cyanomethyl-trimethylphosphonium iodide (prepared according to Tetrahedron 2001, 57, 5451-54). The reaction mixture was heated up to 90° C. for 16 hours. Extraction with ethyl acetate, washing with potassium carbonate solution and water followed by drying and evaporation of the solvent yielded 0.44 g of the title compound as a brownish solid.

¹H-NMR (d6-DMSO): δ=2.35 (s), 3.0-3.2 (m), 3.7 (mc), 5.0 (mc), 6.55 (d), 6.70 (d), 7.20 (d), 7.70 (br s), 8.40 (d).

The following intermediate compounds of the formula II listed in table I below were prepared in an analogous manner.

The compounds of formula I according to the invention as well as intermediates of formula II were characterized by ¹H-NMR or by their melting points.

TABLE I

| | | Physical property |
|---|---|---|
| | | melting point mₚ [° C.] or |
| IC.II. | | chemical shift (¹H-NMR [δ |
| No. | Intermediate compound (structure of formula II) | ppm]) |
| IC.II.1. | 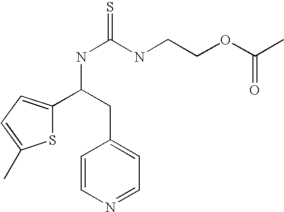 | acetic acid 2-{3-[1-(5- methyl-thiophen-2-yl)-2- pyridin-4-yl-ethyl]- thioureido}-ethyl ester; Cf. data in preparation example P.1 Step 4. |
| IC.II.2. | 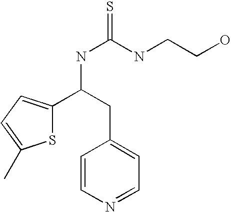 | 1-(2-hydroxy-ethyl)-3-[1- (5-methyl-thiophen-2-yl)- 2-pyridin-4-yl-ethyl]- thiourea; Cf. data in preparation example P.1 Step 5. |
| IC.II.3. | 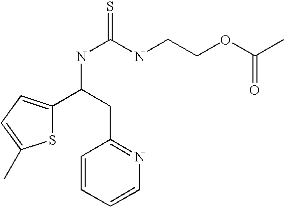 | Viscous oil |
| IC.II.4. | 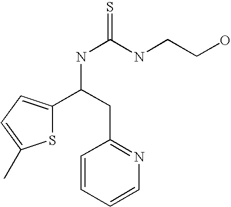 | Dark oil |
| IC.II.5. | 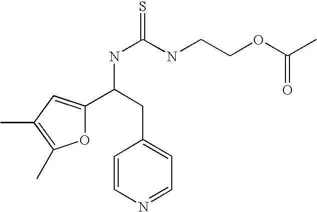 | Viscous oil |
| IC.II.6. | 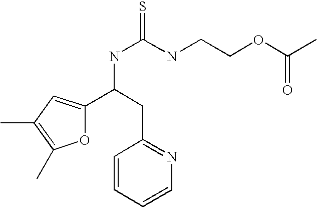 | Viscous oil |
| IC.II.7. | 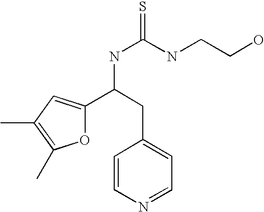 | Viscous oil |
| IC.II.8. | 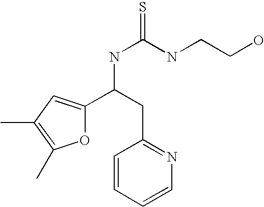 | Viscous oil |
| IC.II.9. | 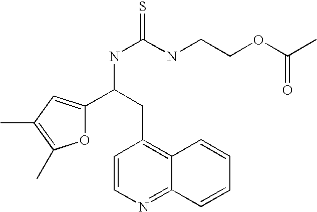 | Yellow oil |
| IC.II.10. | 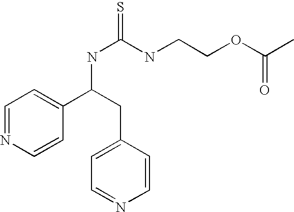 | Viscous oil |
| IC.II.11. | 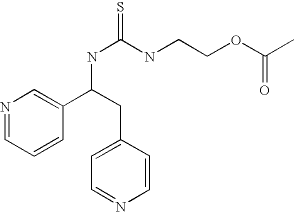 | Viscous oil |
| IC.II.12. | 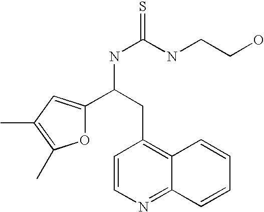 | Viscous oil |
| IC.II.13. | 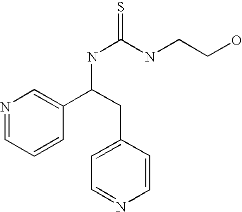 | Viscous oil |
| IC.II.14. | 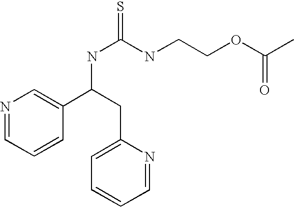 | Viscous oil |
| IC.II.15. | 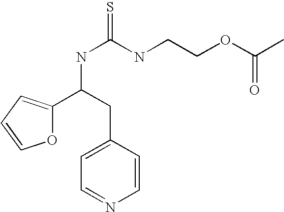 | Viscous oil |
| IC.II.16. | 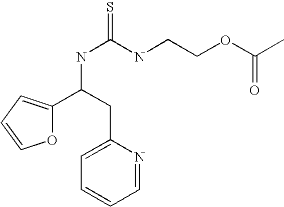 | Viscous oil |
| IC.II.17. | 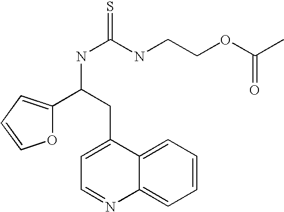 | Viscous oil |
| IC.II.18. | 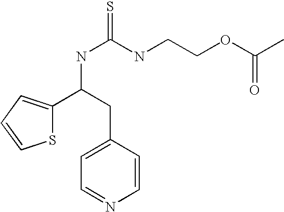 | Viscous oil |
| IC.II.19. | 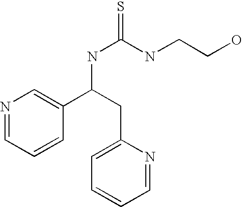 | Viscous oil |
| IC.II.20. | 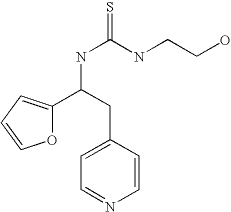 | Viscous oil |
| IC.II.21. | 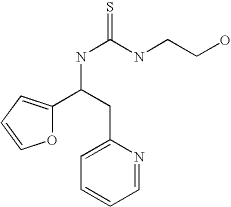 | Viscous oil |
| IC.II.22. | 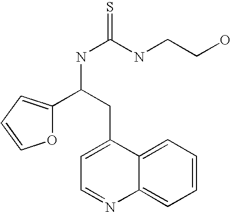 | Viscous oil |
| IC.II.23. | 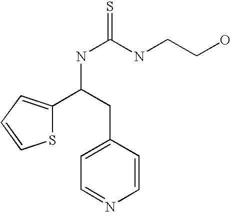 | Viscous oil |
| IC.II.24. | 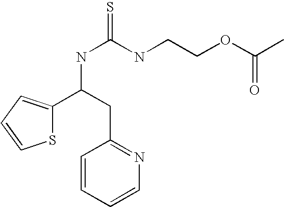 | Viscous oil |
| IC.II.25. | 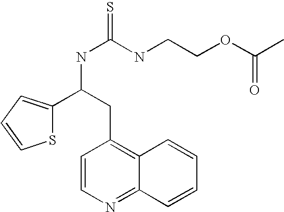 | Viscous oil |
| IC.II.26. | 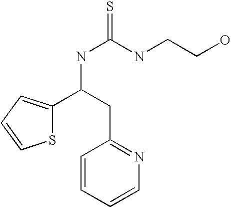 | Viscous oil |
| IC.II.27. | 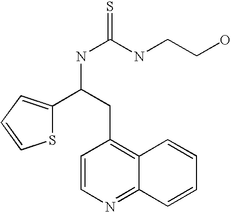 | Viscous oil |
| IC.II.28. | 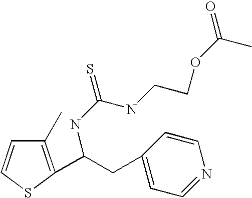 | Viscous oil |
| IC.II.29. | 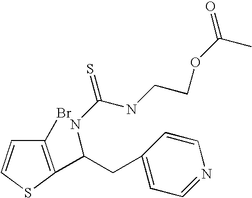 | Viscous oil |
| IC.II.30. | 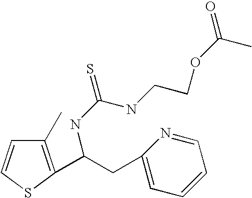 | Viscous oil |
| IC.II.31. | 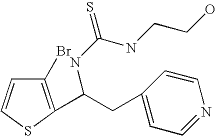 | m.p. 141-140 |
| IC.II.32. | 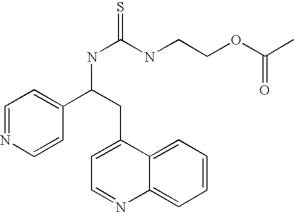 | Viscous oil |
| IC.II.33. | 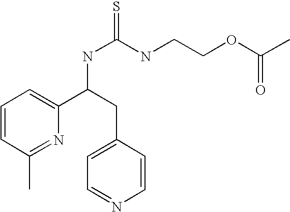 | Viscous oil |
| IC.II.34. | 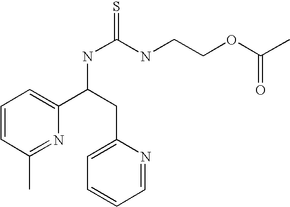 | Viscous oil |
| IC.II.35. | 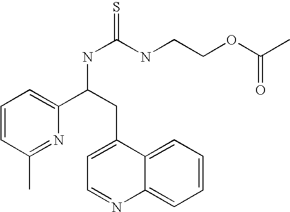 | Viscous oil |
| IC.II.36. | 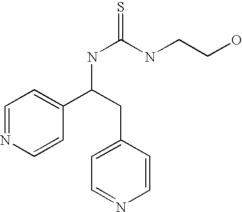 | Viscous oil |
| IC.II.37. | 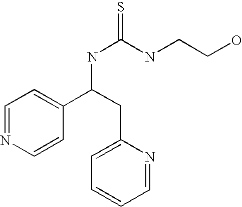 | Viscous oil |
| IC.II.38. | 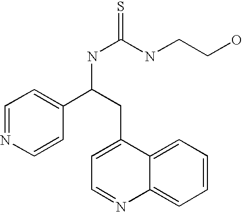 | Viscous oil |
| IC.II.39. | 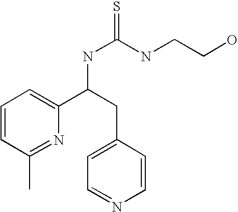 | Viscous oil |
| IC.II.40. | 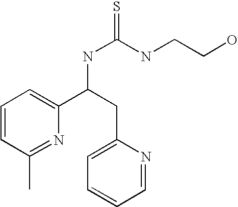 | Viscous oil |
| IC.II.41. | 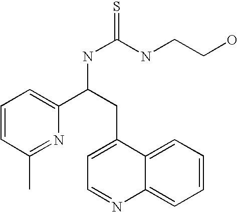 | Viscous oil |

The following compounds of the formula I listed in table II below were also prepared in an analogous manner.

TABLE II

| | | Physical property |
|---|---|---|
| | | (melting point [° C.] or |
| | | chemical shift (¹H-NMR |
| C.I. No. | Compound (structure of formula I) | [δ ppm]) |
| C.I.1. | 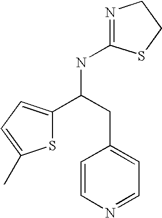 | (4,5-Dihydro-thiazol-2- yl)-[1-(5-methyl- thiophen-2-yl)-2-pyridin- 4-yl-ethyl]-amine; Cf. data in preparation example P.1 Step 6. |
| C.I.2. | 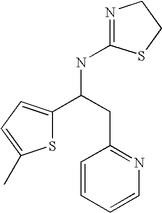 | 2.4 (s), 3.2-3.4 (m), 3.9 (dd), 5.35 (t), 5.45 (br s), 6.5 (d), 6.65 (d), 7.05- 7.15 (m), 7.55 (dd), 8.5 (d) |
| C.I.3. | 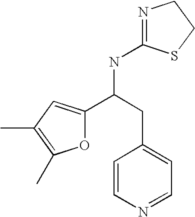 | d₆-DMSO: 1.7 (s), 2.15 (s), 3.05 (mc), 3.20 (mc), 3.75 (mc), 4.85 (mc), 5.95 (s), 7.2 (d), 8.4 (d) |
| C.I.4. | 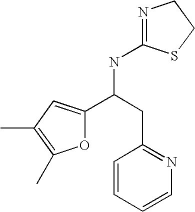 | d₆-DMSO: 1.7 (s), 2.1 (s), 3.15 (mc), 3.7 (mc), 5.15 (mc), 5.9 (s), 7.15 (mc), 7.65 (mc), 8.45 (d) |
| C.I.5. | 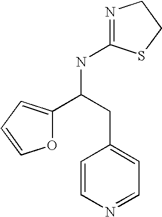 | d₆-DMSO: 3.0-3.2 (m), 3.75 (mc), 5.05 (br s), 6.20 (mc), 6.35 (mc), 7.15 (d), 7.3 (br s), 7.55 (mc), 8.40 (d) |
| C.I.6. | 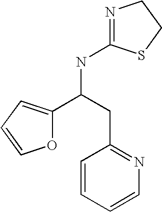 | d₆-DMSO: 3.15 (mc), 3.75 (mc), 5.25 (mc), 6.15 (s), 6.30 (s), 7.2 (mc), 7.3 (br s), 7.5 (s), 7.65 (t), 8.45 (mc) |
| C.I.7. | 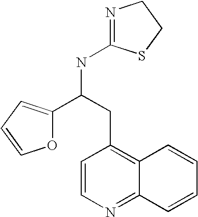 | d₆-DMSO: 3.2 (t), 3.55 (mc), 3.75 (mc), 5.20 (mc), 6.2 (s), 6.35 (s), 7.25 (d), 7.45 (br s), 7.55 (s), 7.6-7.75 (m), 8.0 (d), 8.3 (d), 8.75 (d) |
| C.I.8. | 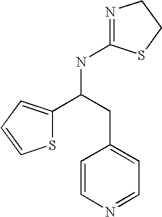 | d₆-DMSO: 3.05-3.2 (m), 3.70 (mc), 5.2 (mc), 6.95 (mc), 7.2-7.45 (m), 8.4 (d) |
| C.I.9. | 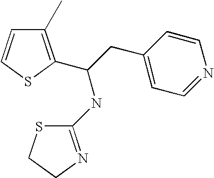 | CDCl₃: 2.0 (s), 3.0 (mc), 3.25-3.4 (m), 3.85 (mc), 5.1 (mc), 6.75 (d), 7.0- 7.15 (m), 8.45 (d). |
| C.I.10. | 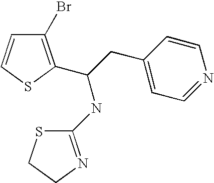 | CDCl₃: 3.15 (mc), 3.25 (mc), 5.00 (mc), 6.90 (d), 7.1 (d), 7.2 (mc), 8.45 (d) |
| C.I.11. | 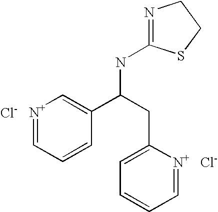 | CD₃OD: 3.60 (mc), 3.85- 4.0 (m), 5.95 (mc), 8.0 (mc), 8.15-8.25 (m), 8.60 (mc), 8.8-8.95 (m), 9.20 (mc) |
| C.I.12. | 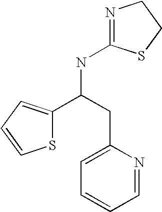 | d₆-DMSO: 3.1-3.25 (m), 3.70 (mc), 5.45 (mc), 6.85 (d), 7.15-7.5 (m), 7.65 (mc), 8.45 (mc) |
| C.I.13. | 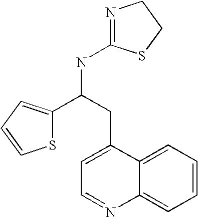 | d₆-DMSO: 3.15 (mc), 3.5-3.8 (m), 5.4 (br s), 6.9-7.05 (m), 7.30-7.75 (m), 8.00 (d), 8.30 (d), 8.70 (mc) |

### B. Biological Examples

### Examples of Action Against Pests

The action of the compounds of the general formulae I against pests was demonstrated by the following experiment:

B.1. Cowpea Aphid (Aphis craccivora)

The active compounds were formulated in 50:50 acetone:water. Potted cowpea plants colonized with 100-150 aphids of various stages were sprayed after the pest population has been recorded. Population reduction was recorded after 24, 72, and 120 hours.

Compound examples no C.I.9 and C.I.12 in table II showed over 80% mortality against cowpea aphid at a concentration of 300 ppm in comparison with untreated controls.

B.2. Green Peach Aphid (Myzus persicae)

Pepper plants in the 2nd leaf-pair stage (variety ‘California Wonder’) were infested with approximately 40 laboratory-reared aphids by placing infested leaf sections on top of the test plants. The leaf sections were removed after 24 hours. The leaves of the intact plants were dipped into gradient solutions of the test compound. Aphid mortality on the treated plants, relative to mortality on check plants, was determined after 5 days.

Compound example no C.I.10 in table II showed over 80% mortality against green-peach aphid at a concentration of 300 ppm in comparison with untreated controls.

An action of the compounds of the general formula I against pests can also be demonstrated by the following experiment:

B.2. Cotton Aphid (Aphis gossypii)

Cotton plants in the cotyledon stage (variety ‘Delta Pine’) are infested with approximately 100 laboratory-reared aphids by placing infested leaf sections on top of the test plants. The leaf sections are removed after 24 hours. The cotyledons of the intact plants are dipped into gradient solutions of the test compound. Aphid mortality on the treated plants, relative to mortality on check plants, is determined after 5 days.

1-64. (canceled)

65. A 1-(Azolin-2-yl)-amino-1,2-heterocyclyl-ethane compound of the general formula I 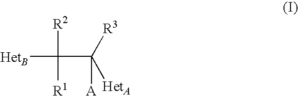
wherein
R¹, R², R³ are, independently of each other, selected from the group consisting of hydrogen, halogen, C₁-C₆-alkyl, C₂-C₆-alkenyl, C₂-C₆-alkynyl, C₁-C₆-haloalkyl, C₃-C₆-cycloalkyl, and C₃-C₆-halocycloalkyl,
wherein 1, 2 or 3 hydrogen atoms in the aforementioned aliphatic radicals may be replaced, independently of one another, by a radical selected from the group consisting of CN, NO₂, OH, SH, NH, CO₂H, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-haloalkoxy and C₁-C₆-alkylthio and
wherein 1, 2, 3, 4 or 5 hydrogen atoms of the C₃-C₆-cycloalkyl, C₃-C₆-halocycloalkyl may be replaced by radicals selected from the group consisting of C₁-C₆-alkyl and C₁-C₆-haloalkyl, phenyl and benzyl, wherein the phenyl ring in the last two mentioned radicals may be unsubstituted or may carry 1, 2, 3, 4 or 5 radicals which are, independently of each other, a radical selected from the group consisting of halogen, C₁-C₆-alkyl, C₁-C₆-haloalkyl, C₁-C₆-alkylthio, C₁-C₆-haloalkylthio, C₁-C₆-alkoxy and C₁-C₆-haloalkoxy;
A is a radical of the formulae A¹ or A²: 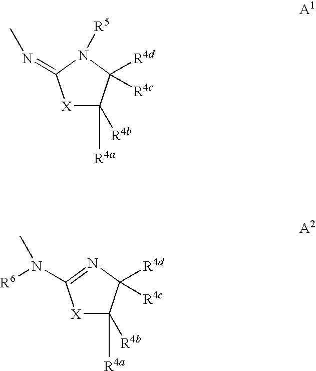
wherein
X is sulfur, oxygen or NR⁷;
R^{4a}, R^{4b}, R^{4c}, R^{4d} are, independently of each other, selected from the group consisting of hydrogen, halogen, C₁-C₆-alkyl, C₁-C₆-haloalkyl, C₁-C₆-alkylamino, C₁-C₆-alkoxy, C₁-C₆-haloalkoxy, C₃-C₆-cycloalkyl and C₃-C₆-halocycloalkyl,
wherein 1, 2 or 3 hydrogen atoms in the aforementioned aliphatic radicals may be replaced, independently of one another, by a radical selected from the group consisting of CN, NO₂, OH, SH, NH₂, CO₂H, C₂-C₆-alkenyl, C₂-C₆-alkynyl, C₁-C₆-alkoxy, C₂-C₆-alkenyloxy, C₂-C₆-alkynyloxy, C₁-C₆-haloalkoxyl and C₁-C₆-alkylthio, and
wherein 1, 2, 3, 4 or 5 hydrogen atoms of the C₃-C₆-cycloalkyl and C₃-C₆-halocycloalkyl may be replaced by radicals selected from the group consisting of C₁-C₆-alkyl and C₁-C₆-haloalkyl;
R⁵, R⁶, R⁷, R⁹ are, independently of each other, selected from the group consisting of hydrogen, CN, NO₂, C₁-C₆-alkyl, C₂-C₆-alkenyl, C₂-C₆-alkynyl, C₃-C₈-cycloalkyl, C₃-C₈-halocycloalkyl, C₁-C₆-alkoxy, (C₁-C₆-alkoxy)methylen, C₁-C₆-alkylthio, C₁-C₆-alkylsulfinyl, C₁-C₆-alkylsulfonyl,
wherein the aliphatic moieties in the aforementioned radicals may be unsubstituted, partially or completely halogenated and/or may carry 1, 2 or 3 radicals, which are independently of one another, selected from the group consisting of CN, NO₂, OH, SH, NH₂, CO₂H, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-haloalkoxy and C₁-C₆-alkylthio and
wherein 1, 2, 3, 4 or 5 hydrogen atoms of the C₃-C₈-cycloalkyl and C₃-C₈-halocycloalkyl may replaced by radicals selected from the group consisting of C₁-C₆-alkyl and C₁-C₆-haloalkyl,
C(O)NR^{a}R^{b}, C(S)NR^{a}R^{b}, (SO₂)NR^{a}R^{b}, C(═O)R^{c} or C(S)R^{c}, phenyl, phenyloxy and benzyl,
wherein the phenyl ring in each of the last three mentioned radicals may be unsubstituted or may carry 1, 2, 3, 4 or 5 radicals, independently of one another selected from the group consisting of halogen, C₁-C₆-alkyl, C₁-C₆-haloalkyl, C₁-C₆-alkylthio, C₁-C₆-haloalkylthio, C₁-C₆-alkoxy and C₁-C₆-haloalkoxy radicals;
Het_{A}, Het_{B} are, independently of each other, selected from the group consisting of a 5-, 6- or 7-membered saturated, partially unsaturated or aromatic heterocyclic ring which contains 1, 2, 3 or 4 heteroatoms selected from the group consisting of oxygen, nitrogen and sulfur as ring members, wherein the heterocyclic ring may optionally be fused to another ring selected from the group consisting of phenyl, a saturated or partially unsaturated 5-, 6-, or 7-membered carbocycle and a 5-, 6- or 7-membered saturated, partially unsaturated or aromatic heterocycle, which contains 1, 2 or 3 heteroatoms selected from the group consisting of oxygen, sulfur and nitrogen atoms as ring members, and
wherein the 5-, 6- or 7-membered heterocyclic ring and/or the respective fused ring may carry at its carbon atoms any combination of m radicals R⁸ and/or may carry at its nitrogen atom, if present, a radical R⁹, which is as defined above or oxygen:
m is 0, 1, 2, 3 or 4,
R⁸ is selected independently from m from the group consisting of halogen, OH, SH, NH₂, SO₃H, COOH, CN, N₃, NO₂, CONH₂, CSNH₂, CH═N—OH, CH═N—O—(C₁-C₆)-alkyl, C₁-C₆-alkyl, C₂-C₆-alkenyl, C₂-C₆-alkynyl, C₃-C₈-cycloalkyl, C₁-C₆-alkylamino, C₂-C₆-alkenylamino, C₂-C₆-alkynylamino, di(C₁-C₆-alkyl)amino, di(C₂-C₆-alkenyl)amino, di(C₂-C₆-alkynyl)amino, C₁-C₆-alkylthio, C₂-C₆-alkenylthio, C₂-C₆-alkynylthio, C₁-C₆-alkylsulfonyl, C₂-C₆-alkenylsulfonyl, C₂-C₆-alkynylsulfonyl, (C₁-C₆-alkyl)carbonyl, (C₂-C₆-alkenyl)-carbonyl, (C₂-C₆-alkynyl)-carbonyl, C₁-C₆-alkoxy, C₂-C₆-alkenyloxy, C₂-C₆-alkynyloxy, (C₁-C₆-alkoxy)carbonyl, (C₂-C₆-alkenyloxy)carbonyl, (C₂-C₆-alkynyloxy)-carbonyl, (C₁-C₆-alkyl)carbonyloxy, (C₂-C₆-alkenyl-)carbonyl-oxy, (C₂-C₆-alkynyl)carbonyloxy, (C₁-C₆-alkyl)carbonyl-amino, (C₂-C₆-alkenyl)carbonyl-amino, (C₂-C₆-alkynyl)carbonyl-amino,
wherein the aliphatic parts of the aforementioned groups may be unsubstituted, partially or completely halogenated and/or may carry any combination of one, two or three radicals, independently of one another selected from the group consisting of CN, NO₂, OH, SH, NH₂, CO₂H, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-haloalkoxy, C₁-C₆-haloalkyl and C₁-C₆-alkylthio;
C(O)NR^{a}R^{b}, C(S)NR^{a}R^{b}, (SO₂)NR^{a}R^{b}, C(═O)R^{c} or C(═S)R^{c},
a radical Y—Ar and a radical Y-Cy, wherein
Y is a single bond, O, S, NH, C₁-C₆-alkandiyl or C₁-C₆-alkanyloxy,
Ar is phenyl, naphthyl or a mono- or bicyclic 5- to 10-membered heteroaromatic ring, which contains 1, 2, 3 or 4 heteroatoms selected from the group consisting of oxygen, sulfur and nitrogen atoms as ring members, wherein Ar is unsubstituted or may carry any combination of 1, 2, 3, 4 or 5 radicals, independently of one another selected from the group consisting of halogen, CN, NO₂, OH, SH, NH₂, CO₂H, C₁-C₆-alkyl, C₁-C₆-haloalkyl, C₁-C₆-alkoxy, C₂-C₆-alkenyloxy, C₂-C₆-alkynyloxy, C₁-C₆-haloalkoxy and C₁-C₆-alkylthio;
Cy is C₃-C₈-cycloalkyl, which is unsubstituted or may carry any combination of 1, 2, 3, 4 or 5 radicals, independently of one another selected from the group consisting of halogen, CN, NO₂, OH, SH, NH₂, CO₂H, C₁-C₆-alkyl, C₁-C₆-haloalkyl, C₁-C₆-alkoxy, C₂-C₆-alkenyloxy, C₂-C₆-alkynyloxy, C₁-C₆-haloalkoxy and C₁-C₆-alkylthio;
and wherein
R^{a} and R^{b} are each independently selected from one another from the group consisting of hydrogen, C₁-C₆-alkyl, C₁-C₆-haloalkyl, C₂-C₆-alkenyl, C₂-C₆-haloalkenyl, C₂-C₆-alkynyl, and C₂-C₆-haloalkynyl, wherein 1, 2 or 3 hydrogen atoms in the aforementioned aliphatic radicals may be replaced, independently of one another, by a radical selected from the group consisting of CN, NO₂, OH, SH, NH₂, CO₂H, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-haloalkoxy, C₁-C₆-haloalkyl and C₁-C₆-alkylthio;
R^{c} is selected from the group consisting of hydrogen, C₁-C₆ alkyl, C₂-C₆-alkenyl, C₂-C₆-alkynyl, C₃-C₈-cycloalkyl, C₁-C₆-alkylthio, C₁-C₆-alkoxy, (C₁-C₆-alkyl)amino, di(C₁-C₆-alkyl)amino, hydrazino, (C₁-C₆-alkyl)hydrazino, di(C₁-C₆-alkyl)hydrazino,
wherein the aliphatic parts of the aforementioned groups may be unsubstituted, partially or completely halogenated or may carry any combination of one, two or three radicals, independently of one another selected from the group consisting of CN, NO₂, OH, SH, NH₂, CO₂H, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-haloalkoxy, C₁-C₆-haloalkyl and C₁-C₆-alkylthio,
phenyl, and a mono- or bicyclic 5- to 10-membered heteroaromatic ring, which contains 1, 2, 3 or 4 heteroatoms selected from the group consisting of oxygen, sulfur and nitrogen atoms as ring members, wherein phenyl and the heteroaromatic ring are unsubstituted or may carry any combination of 1, 2, 3, 4 or 5 radicals, independently of one another selected from the group consisting of halogen, CN, NO₂, OH, SH, NH₂, CO₂H, C₁-C₆-alkyl, C₁-C₆-haloalkyl, C₁-C₆-alkoxy, C₂-C₆-alkenyloxy, C₂-C₆-alkynyloxy, C₁-C₆-haloalkoxy and C₁-C₆-alkylthio;
and the salts, enantiomers or diasteromers thereof.

66. The 1-(Azolin-2-yl)-amino-1,2-heterocyclyl-ethane compound of claim 65, wherein Het_{A} and/or Het_{B} are selected independently from one another from the radicals of the formulae Het.1 to Het.63 as defined herein: 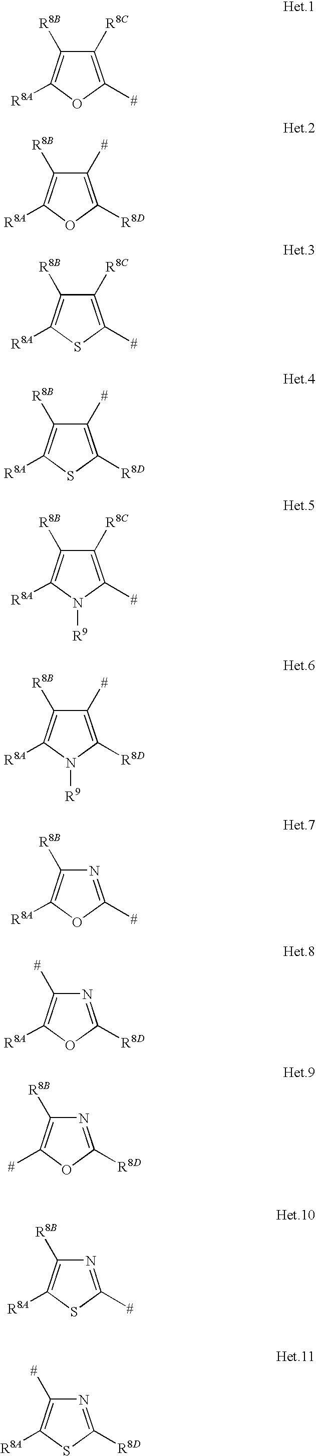 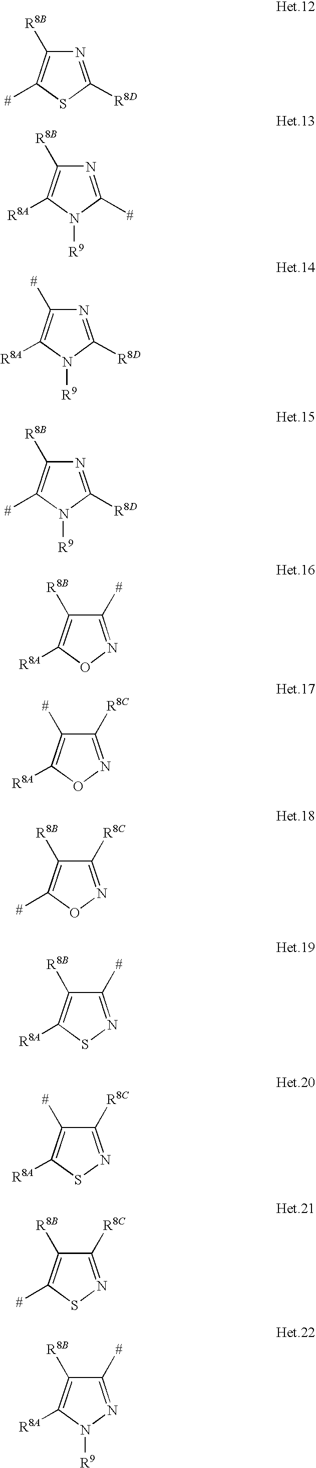 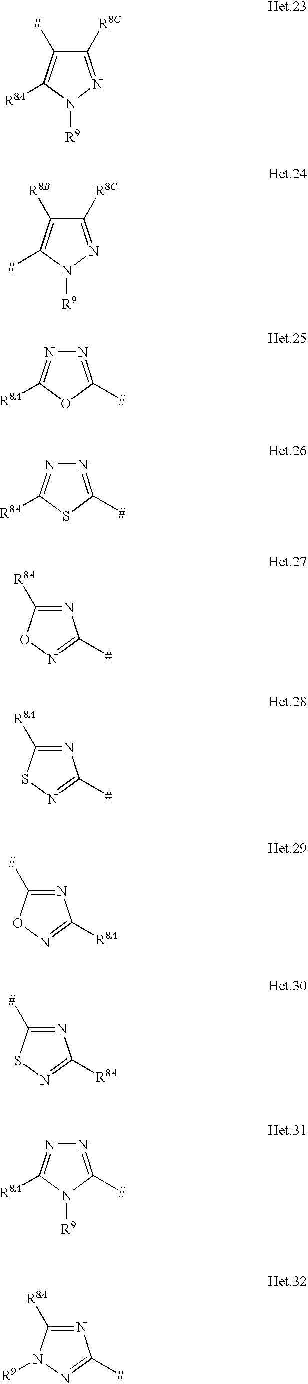 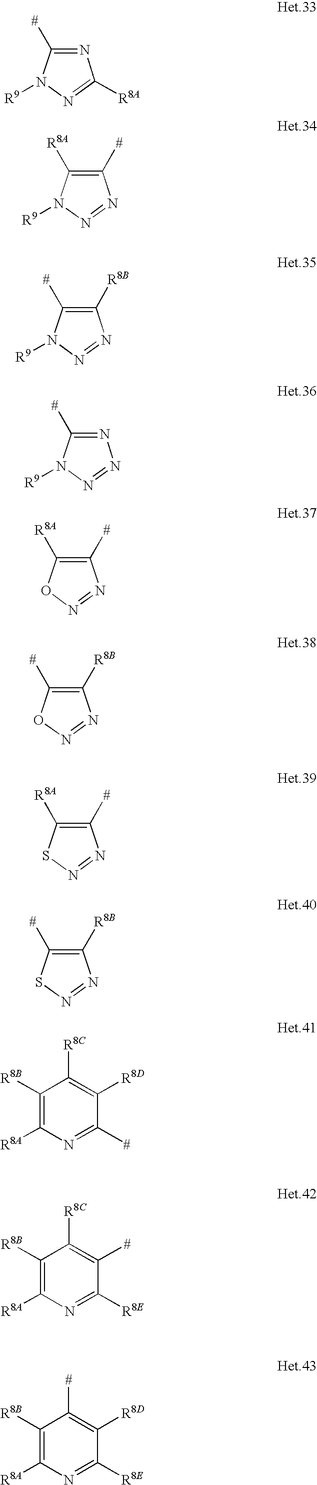 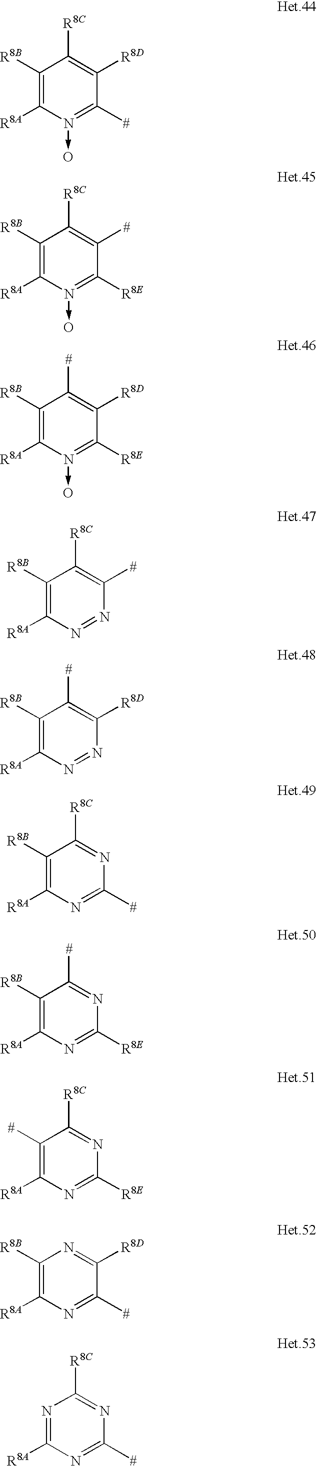 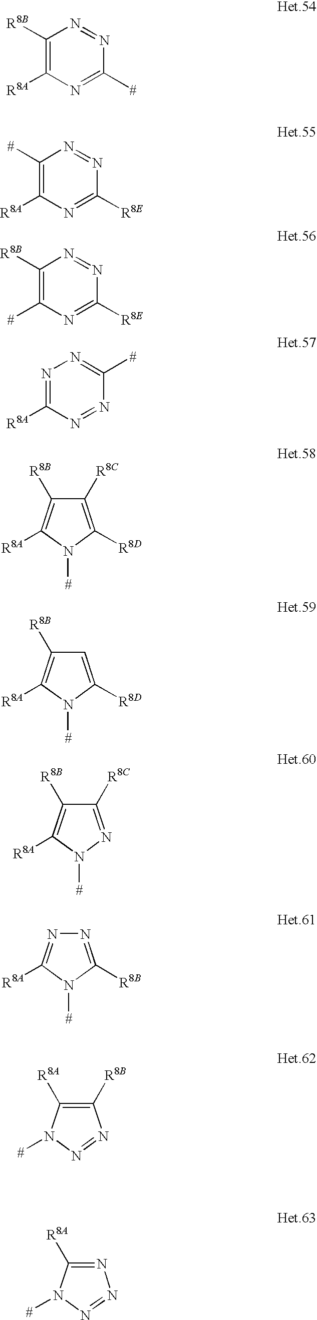
wherein # denotes the position of attachment in formula I, wherein the capital letter A, B, C, D and E joined to R⁸ denotes the position of R⁸ in formula I and wherein R^{8A}, R^{8B}, R^{8C}, R^{8D} and R^{8E}, independently of each other, are hydrogen or have one of the meanings given for R⁸ in claim 65.

67. The 1-(Azolin-2-yl)-amino-1,2-heterocyclyl-ethane compound of claim 66, wherein Het_{A} is selected from the group consisting of the radicals of formulae Het.1-Het.57.

68. The 1-(Azolin-2-yl)-amino-1,2-heterocyclyl-ethane compound of claim 66, wherein Het_{A} and/or Het_{B} are selected independently from each other from the group consisting of the radicals of formulae Het.1, Het.2, Het.3, Het.4, Het.5, Het.6, Het.7, Het.8, Het.9, Het.10, Het.11, Het.12, Het.13, Het.14, Het.15, Het.16, Het.17, Het.18, Het.19, Het.20, Het.21, Het.22, Het.23, Het.24, Het.25, Het.26, Het.27, Het.28, Het.29, Het.30, Het.41, Het.42, Het.43, Het.49, Het.50 and Het.51.

69. The 1-(Azolin-2-yl)-amino-1,2-heterocyclyl-ethane compound of claim 66, wherein Het_{A} is selected from the group consisting of the radicals of formulae Het.1, Het.2, Het.3, Het.4, Het.22, Het.23, Het.24, Het.41, Het.42 and Het.43.

70. The 1-(Azolin-2-yl)-amino-1,2-heterocyclyl-ethane compound of claim 66, wherein Het_{B} is selected from the group consisting of the radicals of formulae Het.1, Het.2, Het.3, Het.4, Het.22, Het.23, Het.24, Het.41, Het.42 and Het.43.

71. The 1-(Azolin-2-yl)-amino-1,2-heterocyclyl-ethane compound of claim 66, wherein Het_{A} is selected from the group consisting of the radicals of formulae Het.1, Het.2, Het.3 and Het.4.

72. The 1-(Azolin-2-yl)-amino-1,2-heterocyclyl-ethane compound of claim 66, wherein Het_{B} is selected from the group consisting of the radicals of formulae Het.1, Het.2, Het.3 and Het.4.

73. The 1-(Azolin-2-yl)-amino-1,2-heterocyclyl-ethane compound of claim 66, wherein
R¹, R², R³ are, independently of each other, selected from the group consisting of hydrogen, halogen, C₁-C₆-alkyl, C₁-C₆-haloalkyl, C₃-C₆-cycloalkyl and C₃-C₆-halocycloalkyl,
wherein 1, 2 or 3 hydrogen atoms in the aforementioned aliphatic radicals may be replaced, independently of one another, by a radical selected from the group consisting of cyano, nitro, hydroxy, mercapto, amino, carboxyl, C₂-C₆-alkenyl, C₂-C₆-alkynyl, C₁-C₆-alkoxy, C₂-C₆-alkenyloxy, C₂-C₆-alkynyloxy, C₁-C₆-haloalkoxy and C₁-C₆-alkylthio, and
wherein 1, 2, 3, 4 or 5 hydrogen atoms of the C₃-C₆-cycloalkyl and C₃-C₆-halocycloalkyl may be replaced by radicals selected from the group consisting of C₁-C₆-alkyl and C₁-C₆-haloalkyl.

74. The 1-(Azolin-2-yl)-amino-1,2-heterocyclyl-ethane compound of claim 65, wherein R¹, R² and R³ are, independently of each other, selected from the group consisting of hydrogen, halogen and C₁-C₆-alkyl.

75. The 1-(Azolin-2-yl)-amino-1,2-heterocyclyl-ethane compound of claim 74, wherein R¹, R² and R³ are, independently of each other, selected from the group consisting of hydrogen and C₁-C₆-alkyl.

76. The 1-(Azolin-2-yl)-amino-1,2-heterocyclyl-ethane compound of claim 74, wherein R¹, R² and R³ are hydrogen.

77. The 1-(Azolin-2-yl)-amino-1,2-heterocyclyl-ethane compound of claim 74, wherein R³ is hydrogen.

78. The 1-(Azolin-2-yl)-amino-1,2-heterocyclyl-ethane compound of claim 65, wherein R^{4a}, R^{4b}, R^{4c}, R^{4d} are, independently of each other, selected from the group consisting of hydrogen, halogen, C₁-C₆-alkyl and C₁-C₆-haloalkyl.

79. The 1-(Azolin-2-yl)-amino-1,2-heterocyclyl-ethane compound of claim 78, wherein R^{4a}, R^{4b}, R^{4c} and R^{4d} are hydrogen.

80. The 1-(Azolin-2-yl)-amino-1,2-heterocyclyl-ethane compound of claim 65, wherein R⁵ or R⁶ is selected from the group consisting of hydrogen, CN, NO₂, C(═O)R^{c}, C(═S)R^{c}, C(O)NR^{a}R^{b}, C(S)NR^{a}R^{b}, C₁-C₆-alkyl, C₂-C₆-alkenyl, C₂-C₆-alkynyl, C₃-C₈-cycloalkyl, (C₁-C₆-alkoxy)methylen, C₁-C₆-alkylsulfanyl, C₁-C₆-alkylsulfinyl and C₁-C₆-alkylsulfonyl,
wherein the aliphatic moieties in the aforementioned radicals may be unsubstituted, partially or completely halogenated and/or may carry 1, 2 or 3 radicals, which are independently of one another, selected from the group consisting of CN, NO₂, OH, SH, NH₂, CO₂H, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-haloalkoxy and C₁-C₆-alkylthio, and
wherein 1, 2, 3, 4 or 5 hydrogen atoms of the C₃-C₈-cycloalkyl may be replaced by radicals selected from the group consisting of C₁-C₆-alkyl and C₁-C₆-haloalkyl.

81. The 1-(Azolin-2-yl)-amino-1,2-heterocyclyl-ethane compound of claim 80, wherein R⁵ or R⁶ is selected from one another from the group consisting of hydrogen, CN, C(═O)R^{c} and C₁-C₆-alkyl.

82. The 1-(Azolin-2-yl)-amino-1,2-heterocyclyl-ethane compound of claim 81, wherein R⁵ or R⁶ is hydrogen.

83. The 1-(Azolin-2-yl)-amino-1,2-heterocyclyl-ethane compound of claim 65, wherein m is different from 0 and wherein R⁸ is selected independently from m from halogen, OH, SH, NH₂, SO₃H, COOH, CN, CONH₂, C(═O)R^{c}, C₁-C₆-alkyl, C₃-C₈-cycloalkyl, C₁-C₆-alkylamino, and di(C₁-C₆-alkyl)amino,
wherein the aliphatic moieties in the aforementioned radicals may be unsubstituted, partially or completely halogenated and/or may carry 1, 2 or 3 radicals, which are independently of one another, selected from the group consisting of CN, NO₂, OH, SH, NH₂, CO₂H, C₂-C₆-alkenyl, C₂-C₆-alkynyl, C₁-C₆-alkoxy, C₂-C₆-alkenyloxy, C₂-C₆-alkynyloxy, C₁-C₆-haloalkoxy and C₁-C₆-alkylthio and
wherein 1, 2, 3, 4 or 5 hydrogen atoms of the C₃-C₈-cycloalkyl may be replaced by radicals selected from the group consisting of C₁-C₆-alkyl and C₁-C₆-haloalkyl.

84. The 1-(Azolin-2-yl)-amino-1,2-heterocyclyl-ethane compound of claim 83, wherein m is different from 0 and R⁸ is selected independently from m from halogen, C₁-C₆-alkyl and C₁-C₆-alkoxy, and wherein the two last-mentioned radicals may be unsubstituted, partially or fully halogenated.

85. The 1-(Azolin-2-yl)-amino-1,2-heterocyclyl-ethane compound of claim 65, wherein X is sulfur.

86. The 1-(Azolin-2-yl)-amino-1,2-heterocyclyl-ethane compound of claim 65, wherein X is oxygen.

87. The 1-(Azolin-2-yl)-amino-1,2-heterocyclyl-ethane compound of claim 65, wherein X is NR⁷.

88. The 1-(Azolin-2-yl)-amino-1,2-heterocyclyl-ethane compound of claim 87, wherein R⁷ is selected from the group consisting of hydrogen, CN, NO₂, C(═O)R^{c}, C(═S)R^{c}, C(O)NR^{a}R^{b}, C(S)NR^{a}R^{b}, C₁-C₆-alkyl, C₂-C₆-alkenyl, C₂-C₆-alkynyl, C₃-C₈-cycloalkyl, (C₁-C₆-alkoxy)methylen, C₁-C₆-alkylsulfanyl, C₁-C₆-alkylsulfinyl and C₁-C₆-alkylsulfonyl,
wherein the aliphatic moieties in the aforementioned radicals may be unsubstituted, partially or completely halogenated and/or may carry 1, 2 or 3 radicals, which are independently of one another, selected from the group consisting of CN, NO₂, OH, SH, NH₂, CO₂H, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-haloalkoxy and C₁-C₆-alkylthio, and
wherein 1, 2, 3, 4 or 5 hydrogen atoms of the C₃-C₈-cycloalkyl may be replaced by radicals selected from the group consisting of C₁-C₆-alkyl and C₁-C₆-haloalkyl.

89. The 1-(Azolin-2-yl)-amino-1,2-heterocyclyl-ethane compound of claim 88, wherein R⁷ is selected from the group consisting of hydrogen, a radical C(═O)R^{c} and C₁-C₆-alkyl.

90. The 1-(Azolin-2-yl)-amino-1,2-heterocyclyl-ethane compound of claim 65, wherein m is 1, 2 or 3.

91. The 1-(Azolin-2-yl)-amino-1,2-heterocyclyl-ethane compound of claim 65, wherein m is 1 or 2.

92. The 1-(Azolin-2-yl)-amino-1,2-heterocyclyl-ethane compound of claim 65, wherein the carbon atom which carries the radical A has S-configuration.

93. The 1-(Azolin-2-yl)-amino-1,2-heterocyclyl-ethane compound of claim 65, wherein the carbon atom which carries the radical A has R-configuration.

94. A composition comprising at least one 1-(azolin-2-yl)-amino-1,2-heterocyclyl-ethane compound of claim 65 and/or a salt, enantiomer or diasteromer thereof and at least one carrier material.

95. An agricultural composition comprising at least one 1-(azolin-2-yl)-amino-1,2-heterocyclyl-ethane compound of claim 65 and/or an enantiomer, diasteromer or an agriculturally useful salt thereof and at least one agriculturally acceptable carrier.

96. A veterinary composition comprising at least one 1-(azolin-2-yl)-amino-1,2-heterocyclyl-ethane compound of claim 65 and/or an enantiomer, diasteromer or a veterinary useful salt thereof and at least one veterinary acceptable carrier.

97. A method for combating animal pests by treating the pests with at least one 1-(azolin-2-yl)-amino-1,2-heterocyclyl-ethane compound of claim 65 and/or a salt, enantiomer or diasteromer thereof.

98. The method of claim 97, wherein the animal pests are insects, arachnids or nematodes.

99. The method of claim 98, wherein the animal pests are selected from insects of the orders Homoptera, Lepidoptera or Coleoptera and arachnids of the order of Acarina.

100. A method of combating animal pests which comprises contacting the animal pests or the environment in which the animal pests live or growing or may live or grow or the materials, plants, seeds, soils, surfaces or spaces to be protected from animal attack or infestation with at least one 1-(azolin-2-yl)-amino-1,2-heterocyclyl-ethane compound of claim 65 and/or a salt, enantiomer or diasteromer thereof.

101. The method of claim 100, wherein the animal pests are insects, arachnids or nematodes.

102. The method of claim 101, wherein the animal pests are selected from insects of the orders Homoptera, Lepidoptera or Coleoptera and arachnids of the order of Acarina.

103. A method for protecting crops from attack or infestation by animal pests, which comprises contacting a crop with at least one 1-(azolin-2-yl)-amino-1,2-heterocyclyl-ethane compound of claim 65 and/or a salt, enantiomer or diasteromer thereof.

104. The method of claim 103, wherein the animal pests are insects, arachnids or nematodes.

105. The method of claim 104, wherein the animal pests are selected from insects of the orders Homoptera, Lepidoptera or Coleoptera and arachnids of the order of Acarina.

106. A method for the protection of seeds from soil insects and of the seedlings' roots and shoots from insects comprising contacting the seeds before sowing and/or after pregermination with at least one 1-(azolin-2-yl)-amino-1,2-heterocyclyl-ethane compound of claim 65 and/or a salt, enantiomer or diasteromer thereof.

107. The method of claim 106, wherein the at least one compound is applied in an amount of from 0.1 g to 10 kg per 100 kg of seeds.

108. The method of claim 107, wherein the roots and shoots of the resulting plants are protected.

109. The method of claim 108, wherein the shoots of the resulting plants are protected from insects of the orders Homoptera.

110. A seed comprising at least one 1-(azolin-2-yl)-amino-1,2-heterocyclyl-ethane compound of claim 65 and/or a salt, enantiomer or diasteromer thereof.

111. A method for protecting animals against infestation or infection by parasites which comprises administering a parasitically effective amount of at least one 1-(azolin-2-yl)-amino-1,2-heterocyclyl-ethane compound of claim 65 and/or a salt, enantiomer or diasteromer thereof to the animal in need thereof.

112. A method for treating animals infested or infected by parasites which comprises administering a parasitically effective amount of at least one 1-(azolin-2-yl)-amino-1,2-heterocyclyl-ethane compound of claim 65 and/or a salt, enantiomer or diasteromer thereof to the animal in need thereof.

113. The compound of claim 65 having the general formula II 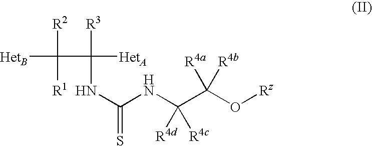
wherein R^{z} is hydrogen or acetyl, and
R^{4a}, R^{4b}, R^{4c}, R^{4d} are, independently of each other, selected from the group consisting of hydrogen, halogen, C₁-C₆-alkyl, C₁-C₆-haloalkyl, C₁-C₆-alkylamino, C₁-C₆-alkoxy, C₁-C₆-haloalkoxy, C₃-C₆-cycloalkyl and C₃-C₆-halocycloalkyl,
wherein 1, 2 or 3 hydrogen atoms in the aforementioned aliphatic radicals may be replaced, independently of one another, by a radical selected from the group consisting of CN, NO₂, OH, SH, NH₂, CO₂H, C₂-C₆-alkenyl, C₂-C₆-alkynyl, C₁-C₆-alkoxy, C₂-C₆-alkenyloxy, C₂-C₆-alkynyloxy, C₁-C₆-haloalkoxyl and C₁-C₆-alkylthio, and
wherein 1, 2, 3, 4 or 5 hydrogen atoms of the C₃-C₆-cycloalkyl and C₃-C₆-halocycloalkyl may be replaced by radicals selected from the group consisting of C₁-C₆-alkyl and C₁-C₆-haloalkyl;
and/or a salt, enantiomer or diasteromer thereof.

114. A composition comprising at least one compound of claim 113 and/or a salt, enantiomer or diasteromer thereof and at least one carrier material.

115. An agricultural composition comprising at least one compound of claim 113 and/or an agriculturally useful salt, enantiomer or diasteromer thereof and at least one agriculturally acceptable carrier.

116. A veterinary composition comprising at least one compound of claim 113 and/or a veterinary useful salt, enantiomer or diasteromer thereof and at least one veterinary acceptable carrier.

117. A method for combating animal pests by treating the pests with at least one compound of claim 113 and/or a salt, enantiomer or diasteromer thereof.

118. A method of combating animal pests which comprises contacting the animal pests or the environment in which the animal pests live or growing or may live or grow or the materials, plants, seeds, soils, surfaces or spaces to be protected from animal attack or infestation with at least one compound of claim 113 and/or a salt, enantiomer or diasteromer thereof.

119. A method for protecting crops from attack or infestation by animal pests, which comprises contacting a crop with at least one compound of claim 113 and/or a salt, enantiomer or diasteromer thereof.

120. A method for the protection of seeds from soil insects and of the seedlings' roots and shoots from insects comprising contacting the seeds before sowing and/or after pregermination with at least one compound of claim 113 and/or a salt, enantiomer or diasteromer thereof.

121. The method of claim 120, wherein the roots and shoots of the resulting plants are protected.

122. The method of claim 120, wherein the shoots of the resulting plants are protected from insects of the order Homoptera

123. A seed comprising at least one compound of claim 113 and/or a salt, enantiomer or diasteromer thereof.

124. A method for protecting animals against infestation or infection by parasites which comprises administering a parasitically effective amount of at least one compound of claim 113 and/or a salt, enantiomer or diasteromer thereof to the animal in need thereof.
A method for treating animals infested or infected by parasites which comprises administering a parasitically effective amount of at least one compound of claim 113 and/or a salt, enantiomer or diasteromer thereof to the animal in need thereof.
